(19) United States
(12) Patent Application Publication
(10) Pub. No.: US 2012/0077793 A1
(43) Pub. Date: Mar. 29, 2012

(54) Compounds for the Treatment of Metabolic Disorders

(75) Inventors: Oscar Barba, Oxford (GB); Peter Timothy Fry, Oxford (GB); Matthew Colin Thor Fyfe, Oxford (GB); William Gattrell, Oxford (GB); Revathy Perpetua Jeevaratnam, Oxford (GB); Thomas Martin Krulle, Oxford (GB); Martin James Procter, Oxford (GB); Colin Peter Sambrook-Smith, Oxford (GB); Karen Lesley Schofield, Oxford (GB); Donald Smyth, Oxford (GB); Alan John William Stewart, Oxford (GB); David French Stonehouse, Oxford (GB); Simon Andrew Swain, Oxford (GB)

(73) Assignee: PROSIDION LIMITED, Oxford (GB)

(21) Appl. No.: 13/255,525

(22) PCT Filed: Mar. 12, 2010
(86) PCT No.: PCT/GB10/50440

§ 371 Date: Nov. 29, 2011

(30) Foreign Application Priority Data
Mar. 12, 2009 (GB) 0904284.7

(51) Int. Cl. A61K 31/4439; A61K 31/4545; C07D 401/12; A61K 31/454; A61K 31/497; A61P 9/12; A61K 31/4725; A61P 3/10; A61P 3/04; A61P 3/00; A61P 3/08; A61P 3/06; C07D 401/14; C07D 413/14

(52) U.S. Cl. 514/210.2; 546/194; 514/318; 546/279.1; 546/208; 514/326; 544/405; 514/255.05; 546/145; 514/307

(57) ABSTRACT

The present invention is directed to therapeutic compounds which have activity as agonists of GPR119 and are useful for the treatment of metabolic disorders including type II diabetes.

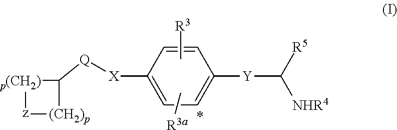

### BACKGROUND OF THE INVENTION

The present invention is directed to therapeutic compounds useful for the treatment of metabolic disorders including type II diabetes. In particular, the present invention is directed to compounds which have activity as agonists of GPR119.

Drugs aimed at the pathophysiology associated with non-insulin dependent type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) places people at high risk of coronary artery disease, and is characterized by a cluster of risk factors including central obesity (excessive fat tissue in the abdominal region), glucose intolerance, high triglycerides and low HDL cholesterol, and high blood pressure. Myocardial ischemia and microvascular disease is an established morbidity associated with untreated or poorly controlled metabolic syndrome.

Obesity is characterized by an excessive adipose tissue mass relative to body size. Clinically, body fat mass is estimated by the body mass index (BMI; weight (kg)/height (m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

There is a continuing need for novel antidiabetic agents, particularly ones that are well tolerated with few adverse effects and in particular for agents which are weight neutral or preferably cause weight loss.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of diabetes.

GPR119 agonists have been shown to stimulate the release of GLP-1 from the GI tract. In doing so, GPR119 agonists (1) enhance glucose-dependent insulin release from the pancreas leading to improvements in oral glucose tolerance; (2) attenuate disease progression by increasing 3-cell cAMP concentrations; and (3) induce weight loss possibly through GLP-1's ability to reduce food intake.

International Patent Applications WO2005/061489, WO2006/070208, WO2006/067532, WO2006/067531, WO2007/003960, WO2007/003961, WO2007/003962, WO2007/003964, WO2007/116229, WO2007/116230, WO2007/138362, WO2008/081204, WO2008/081205, WO2008/081206, WO2008/081207, WO2008/081208, WO2009/050522, WO2009/050971, WO2010/004343, WO2010/004344, WO2010/004345, WO2010/004347 and WO2010/00166 disclose GPR119 receptor agonists.

Dipeptidyl peptidase IV (DPP-IV) is a ubiquitous, yet highly specific, serine protease that cleaves N-terminal dipeptides from polypeptides with L-proline or L-alanine at the penultimate position. Studies with DPP-IV inhibitors show the principle role of DPP-IV is in the inactivation GLP-1. By extending the duration of action of GLP-1, insulin secretion is stimulated, glucagon release inhibited, and gastric emptying slowed. DPP-IV inhibitors are of use for the treatment of type II diabetes, examples of DPP-IV inhibitors include vildagliptin, sitagliptin, alogliptin and saxagliptin.

The possibility of using a combination of a GPR119 agonist and a DPP-IV inhibitor has been suggested, however this requires the administration of two separately formulated products to the patient or the co-formulation of two active ingredients with the inherent problems of achieving compatibility in the physicochemical, pharmacokinetic and pharmacodynamic properties of the two active ingredients. International Patent Application WO2009/034388, published after the priority date of the present application, discloses compounds having dual activity as agonists of GPR119 and inhibitors of DPP-IV.

The compounds of the invention may also have dual activity as agonists of GPR119 and inhibitors of DPP-IV.

### SUMMARY OF THE INVENTION

The present invention is directed to compounds which have activity as agonists of GPR119 and may also be inhibitors of DPP-IV and are useful for the treatment of metabolic disorders including type II diabetes.

### DETAILED DESCRIPTION OF THE INVENTION

The present invention provides compounds of formula (I) and pharmaceutically acceptable salts thereof:

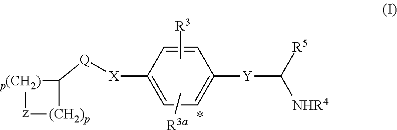

wherein p is 1 or 2;

when p is 2, Z is CHR¹ or NR²; and when p is 1, Z is —N—CH₂-Ph wherein the Ph is optionally substituted by 1 or 2 groups independently selected from C₁₋₄alkyl, C₁₋₄haloalkyl and halo;

R¹ is —N(CH₃)—C(O)—O—C₂₋₄alkyl or —N(CH₃)—C(O)—O—C₃₋₆cycloalkyl wherein the cycloalkyl is optionally substituted by C₁₋₄alkyl;

R² is —C(O)—O—C₂₋₄ alkyl, —C(O)—O—C₃₋₆cycloalkyl wherein the cycloalkyl is optionally substituted by C₁₋₄alkyl, —C(O)—C₂₋₄ alkyl, —C(O)—C₃₋₆cycloalkyl wherein the cycloalkyl is optionally substituted by C₁₋₄alkyl, or R² is:

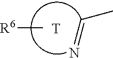

where T together with the —N═C— to which it is attached forms a 5- or 6-membered heteroaryl ring optionally containing up to 2 additional heteroatoms selected from N, O and S;

when T together with the —N═C— to which it is attached forms a 5-membered heteroaryl ring, R⁶ is C₂₋₄ alkyl or C₃₋₆ cycloalkyl optionally substituted by C₁₋₄alkyl, and when T together with the —N═C— to which it is attached forms a 6-membered heteroaryl ring, R⁶ is C₂₋₄ alkyl, fluoro or chloro;

Q is —O—, —O—CR⁸H— or —CR⁸H—O—;

X is phenyl or a 5- or 6-membered heteroaryl group containing one or more heteroatoms selected from N, O and S; provided that when Q is —O—CR⁸H— then X is not a 6-membered heteroaryl group;

Y is a bond, —CH₂— or —CHMe-;

R³ and R^{3a} are independently selected from hydrogen, fluoro or chloro, or when R⁷ is cyano, R³ may be methyl; provided that when Y is a bond, and R³ and R^{3a} are in the ortho position to the Y group they are both hydrogen;

R⁴ is hydrogen or, when Y is —CH₂— or —CHMe-, R⁴ can be —CH₂— linked to position * on the phenyl ring to form a fused 6-membered N-containing heterocycle;

R⁵ is benzyl optionally substituted by one or more fluoro, chloro, cyano or methyl groups, or R⁵ is:

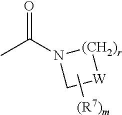

where r is 1 or 2 and m is 0, 1 or 2;

W is CH₂ or, when r is 2, W may be S;

when W is CH₂, R⁷ is fluoro or cyano, and when W is S, R⁷ is cyano; and

R⁸ is hydrogen or methyl.

In a preferred embodiment the compounds of the invention have the stereochemistry as defined in formula (Ia), such compounds demonstrate DPP-IV inhibitory activity:

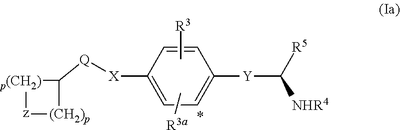

In one of embodiment of the invention each p is independently 1 or 2, i.e. forming a 4-, 5- or 6-membered ring. In another embodiment of the invention each p is the same, i.e. forming a 4- or 6-membered ring. In the compounds of the invention p is preferably 2.

Z is preferably NR².

In one embodiment of the invention R² is —C(O)OR⁴. In a further embodiment of the invention R² is:

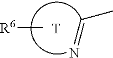

When R² is:

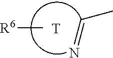

particular 5- or 6-membered heteroaryl rings formed by T together with the —N═C— to which it is attached which may be mentioned are oxadiazole and pyrimidine.

Q is preferably —O— or —CR⁸H—O—, more preferably —CR⁸H—O—.

X is preferably a meta- or para-linked phenyl or a meta or para linked 6-membered heteroaromatic ring containing one or two nitrogen atoms, more preferably a para-linked phenyl or a para linked 6-membered heteroaromatic ring containing one or two nitrogen atoms.

X is preferably phenyl or pyridyl.

R³ is preferably fluoro.

R⁴ is preferably hydrogen.

R⁵ is preferably:

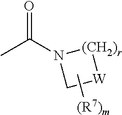

W is preferably CH₂.

r is preferably 2.

While the preferred groups for each variable have generally been listed above separately for each variable, preferred compounds of this invention include those in which several or each variable in formula (I) is selected from the preferred groups for each variable. Therefore, this invention is intended to include all combinations of preferred listed groups.

Representative compounds of the invention which may be mentioned are those provided in the Examples as the free base or a pharmaceutically acceptable salt thereof.

The molecular weight of the compounds of the invention is preferably less than 800, more preferably less than 600.

As used herein, unless stated otherwise, “alkyl” means carbon chains which may be linear or branched. Examples of alkyl groups include ethyl, propyl, isopropyl, butyl, sec- and tert-butyl.

The term “heteroaryl” rings means 5- or 6-membered N-containing heteroaryl rings containing up to 2 additional heteroatoms selected from N, O and S. Examples of such heteroaryl rings are pyrrolyl, pyrazolyl, imidazolyl, oxazolyl, isoxazolyl, thiazolyl, isothiazolyl, triazolyl, oxadiazolyl, thiadiazolyl, pyridinyl, pyridazinyl, pyrimidinyl, pyrazinyl and triazinyl.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The present invention includes all stereoisomers of the compounds of the invention and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of the invention exists, the present invention includes any possible tautomers and pharmaceutically acceptable salts thereof, and mixtures thereof, except where specifically drawn or stated otherwise.

When the compound of the invention and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like

Since the compounds of the invention are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, wherein R¹, R², R³, R^{3a}, R⁵, R⁶, R⁷, R⁸, X, Y, W, Q, Z, m, p and r are as defined for formula (I). PG is a protecting group, Hal is halogen and Tf is triflate

Compounds of formula (I) where p is 2, Q is —O— or —CR⁸H—O—, X is a 2-pyridyl or 2-pyrimidyl and R² is not —C(O)—O—C₂₋₄ alkyl can be synthesized as outlined in Scheme 1. Compounds of formula (IV) can be synthesized by reaction of triflate of formula (II) with a boronate of formula (III) under, for example, Suzuki conditions using [1,1-bis(diphenylphosphino)ferrocene]dichloropalladium in a suitable solvent such as DMF/water at 80° C. Compounds of formula (VI) can be prepared by reaction of aryl halide of formula (IV) with alcohols of formula (V) under standard conditions, such as KO^{t}Bu in a suitable solvent such as THF at 150° C. in a microwave reactor. Deprotection of the amine functionality, using standard conditions well known to those with skill in the art, affords compounds of formula (I) as described above.

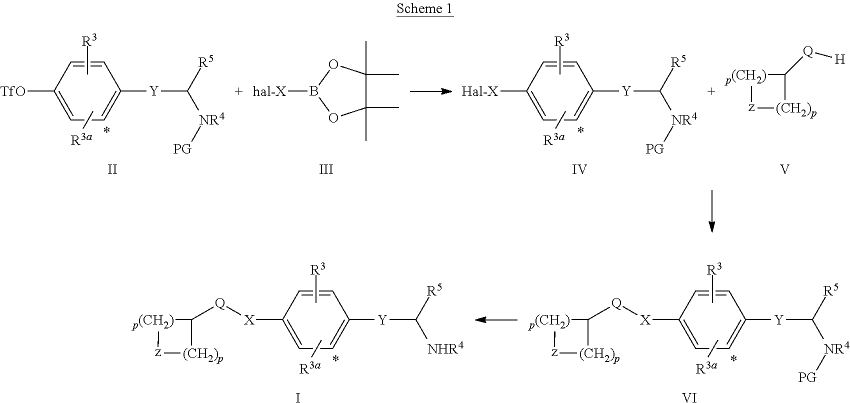

Compounds of formula (I) where p is 2, Q is —O— or —CR⁸H—O—, X is 2-pyridyl or 2-pyrimidyl and R² is —C(O)—O—C₂₋₄ alkyl can be synthesized as outlined in Scheme 2. Aryl bromide of formula (VIII) can be prepared by reaction of alcohol of formula (V) with aryl chloride of formula (VII) in the presence on a suitable base, such as NaH in a suitable solvent such as THF at 60° C. Aryl boronates of formula (IX) can be prepared by reaction of aryl bromide of formula (VIII) and bis(pinacolato)diboron in the presence of a suitable catalyst, such as [1,1-bis(diphenylphosphino)ferrocene]dichloropalladium in a suitable solvent such as 1,4-dioxane at 110° C. Compounds of formula (VI) can be prepared by reaction of triflate of formula (II) with a boronate of formula (IX) under, for example, Suzuki conditions using [1,1-bis(diphenylphosphino)ferrocene]dichloropalladium in a suitable solvent such as DMF/water at 80° C. Deprotection of the amine functionality, using standard conditions well known to those with skill in the art, affords compounds of formula (I) as described above.

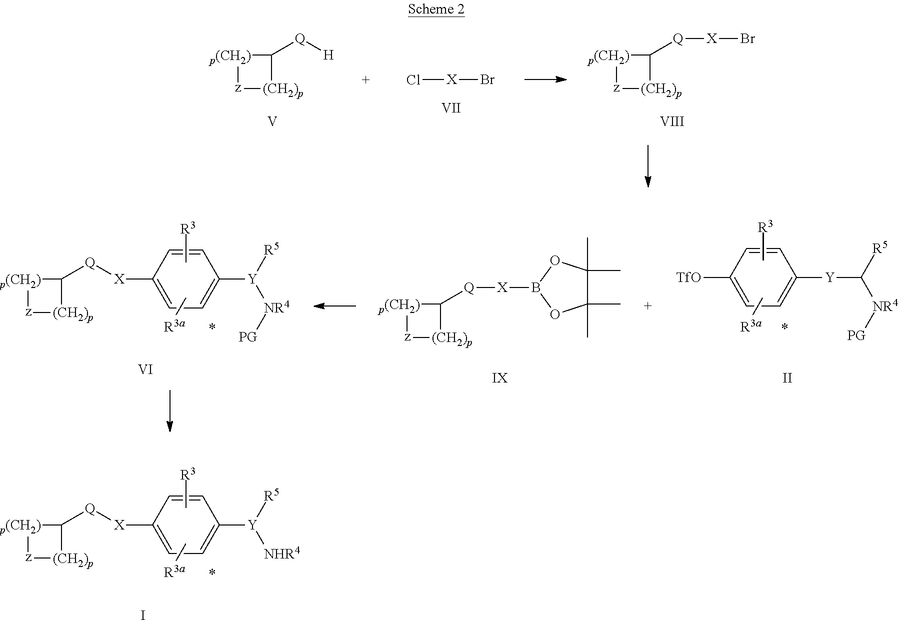

Compounds of formula (I) where p is 2, Q is —O— or —CR⁸H—O— and X is phenyl can be synthesized as outlined in Scheme 3. Aryl halide of formula (XI) can be prepared by reaction of alcohol of formula (V) and phenol (X) under, for example, Mitsunobu conditions using azodicarboxylic dipiperidide and tributylphosphine. Aryl boronates of formula (XII) can be prepared by reaction of aryl halide of formula (XI) and bis(pinacolato)diboron in the presence of a suitable catalyst, such as [1,1-bis(diphenylphosphino)ferrocene]dichloropalladium in a suitable solvent such as 1,4-dioxane at 110° C. Compounds of formula (VI) can be prepared by reaction of triflate of formula (II) with a boronate of formula (XII) under, for example, Suzuki conditions using [1,1-bis(diphenylphosphino)ferrocene]dichloropalladium in a suitable solvent such as DMF/water at 80° C. in a microwave reactor. Deprotection of the amine functionality, using standard conditions well known to those with skill in the art, affords compounds of formula (I) as described above.

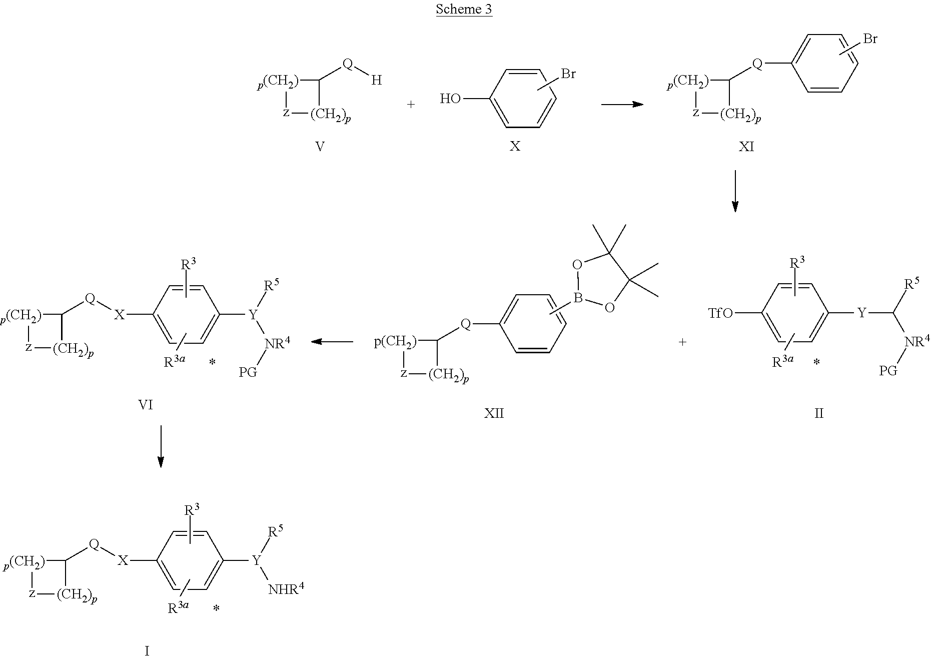

Compounds of formula (I) where p is 2, Q is —O— or —CR⁸H—O— and X is 5-pyridyl or 5-pyrimidyl and can be synthesized as outlined in Scheme 4. Aryl bromide of formula (VIII) can be prepared by reaction of alcohol of formula (V) with aryl bromide of formula (XIII) under, for example, Mitsunobu conditions using azodicarboxylic dipiperidide and tributylphosphine. Compounds of formula (VI) can be prepared by reaction of aryl bromide of formula (VIII) with a boronate of formula (XIV) under, for example, Suzuki conditions using [1,1-bis(diphenylphosphino)ferrocene]dichloropalladium in a suitable solvent such as DMF/water at 80° C. in a microwave reactor. Deprotection of the amine functionality, using standard conditions well known to those with skill in the art, affords compounds of formula (I) as described above.

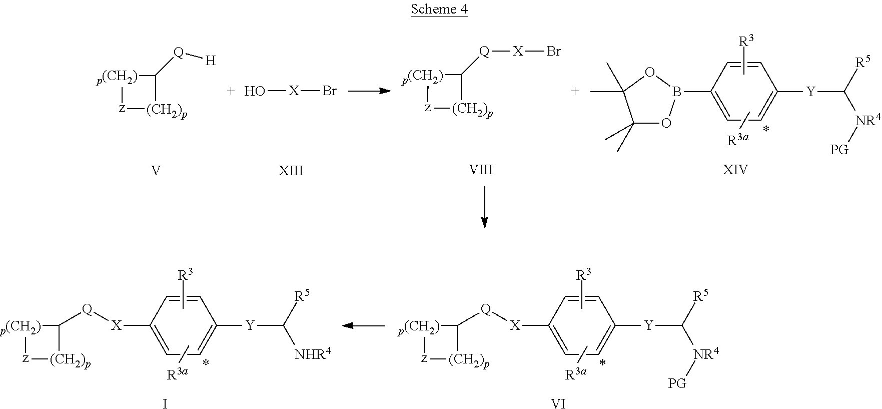

Compounds of formula (I) where p is 2, Q is —O—CR⁸H— and X is phenyl can be synthesized as outlined in Scheme 5. Mesylates of formula (XVI) can be prepared by reaction of alcohol of formula (XV) with methanesulfonyl chloride in the presence of a suitable base, such as triethylamine, in a suitable solvent, such as DCM. Aryl bromides of formula (XI) can be prepared by reaction of mesylates of formula (XVI) with alcohols of formula (XVII) in the presence of a suitable base, such as NaH, in a suitable solvent, such as THF. Aryl boronates of formula (XII) can be prepared by reaction of aryl halide of formula (XI) and bis(pinacolato)diboron in the presence of a suitable catalyst, such as [1,1-bis(diphenylphosphino)ferrocene]dichloropalladium in a suitable solvent such as 1,4-dioxane at 110° C. Compounds of formula (VI) can be prepared by reaction of triflate of formula (II) with a boronate of formula (XII) under, for example, Suzuki conditions using [1,1-bis(diphenylphosphino)ferrocene]dichloropalladium in a suitable solvent such as DMF/water at 80° C. in a microwave reactor. Deprotection of the amine functionality, using standard conditions well known to those with skill in the art, affords compounds of formula (I) as described above.

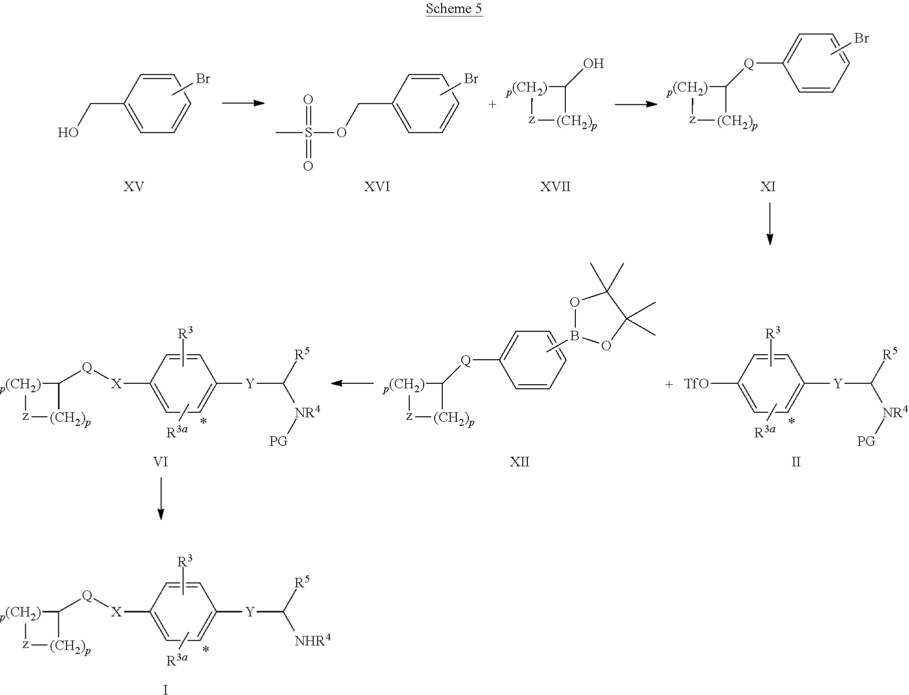

Compounds of formula (I) where p is 1 and X is 2-pyridyl or 2-pyrimidyl can be synthesized as outlined in Scheme 6. Aryl bromide of formula (VIII) can be prepared by reaction of alcohol of formula (V) with aryl bromide of formula (VII) in the presence of a suitable base, such as NaH, in a suitable solvent, such as DMF at 60° C. Compounds of formula (VI) can be prepared by reaction of aryl bromide of formula (VIII) with a boronate of formula (XIV) under, for example, Suzuki conditions using [1,1-bis(diphenylphosphino)ferrocene]dichloropalladium in a suitable solvent such as DMF/water at 80° C. in a microwave reactor. Deprotection of the amine functionality, using standard conditions well known to those with skill in the art, affords compounds of formula (I) as described above.

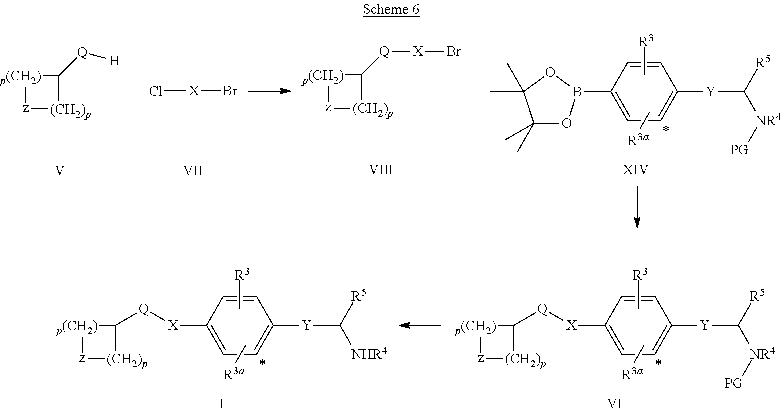

Compounds of formula (I) where p is 1 and X is phenyl, 5-pyridyl or 5-pyrimidyl can be synthesized as outlined in Scheme 7. Aryl bromide of formula (VIII) can be prepared by reaction of alcohol of formula (V) with aryl bromide of formula (XIII) under, for example, Mitsunobu conditions using azodicarboxylic dipiperidide and tributylphosphine. Compounds of formula (VI) can be prepared by reaction of aryl bromide of formula (VIII) with a boronate of formula (XIV) under, for example, Suzuki conditions using [1,1-bis(diphenylphosphino)ferrocene]dichloropalladium in a suitable solvent such as DMF/water at 80° C. in a microwave reactor. Deprotection of the amine functionality, using standard conditions well known to those with skill in the art, affords compounds of formula (I) as described above.

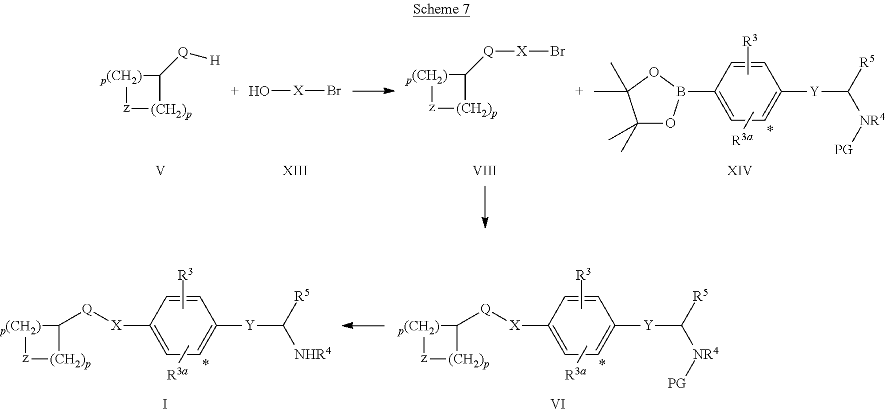

Compounds of formula (I) where p is 2, Q is —O—CR⁸H— and X is oxadiazol-3-yl can be synthesized as outlined in Scheme 8. Nitrile of formula (XVIII) can be prepared by reaction of triflate of formula (II) with ZnCN in the presence of a suitable catalyst, such as [1,1-bis(diphenylphosphino)ferrocene]dichloropalladium in a suitable solvent such as DMF at 70° C. Amidoxime of formula (XIX) can be prepared by reaction of nitrile of formula (XVIII) and hydroxylamine hydrochloride in the presence of a suitable base such as K₂CO₃ in a suitable solvent such as ethanol/water at 78° C. Compounds of formula (VI) can be prepared by reaction of amidoxime of formula (XIX) with acid of formula (XX) under standard conditions, such as isobutyl chloroformate and triethylamine, in a suitable solvent such as DMF. Deprotection of the amine functionality, using standard conditions well known to those with skill in the art, affords compounds of formula (I) as described above.

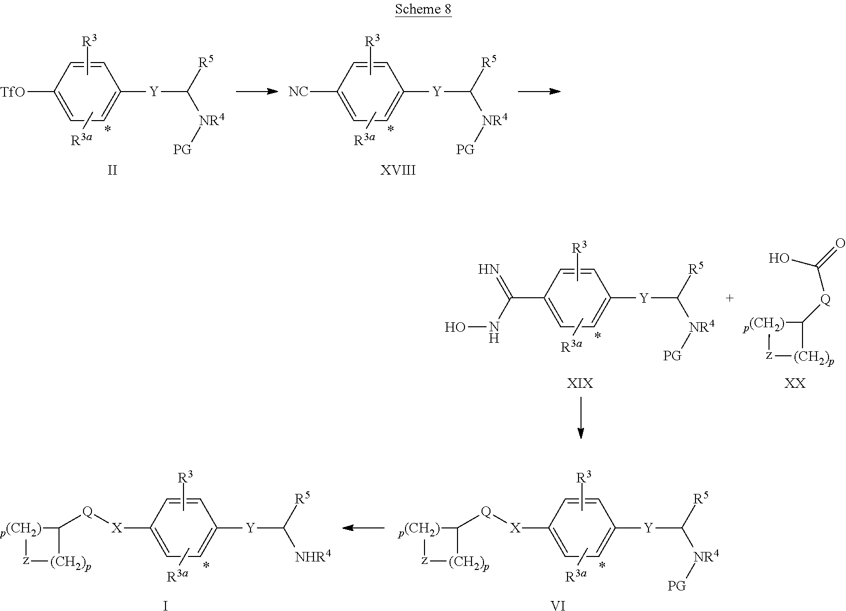

Compounds of formula (I) where p is 2, Q is —O—CR⁸H— and X is oxadiazol-5-yl can be prepared as outlined in Scheme 9. Acid of formula (XXI) can be prepared by reaction of triflate of formula (II) with carbon monoxide in the presence of a suitable catalyst, such as palladium acetate in a suitable solvent, such as DMF at 80° C. Compounds of formula (VI) can be prepared by reaction of acid of formula (XXI) with amidoxime of formula (XXII) under standard conditions, such as isobutyl chloroformate and triethylamine, in a suitable solvent such as DMF. Deprotection of the amine functionality, using standard conditions well known to those with skill in the art, affords compounds of formula (I) as described above.

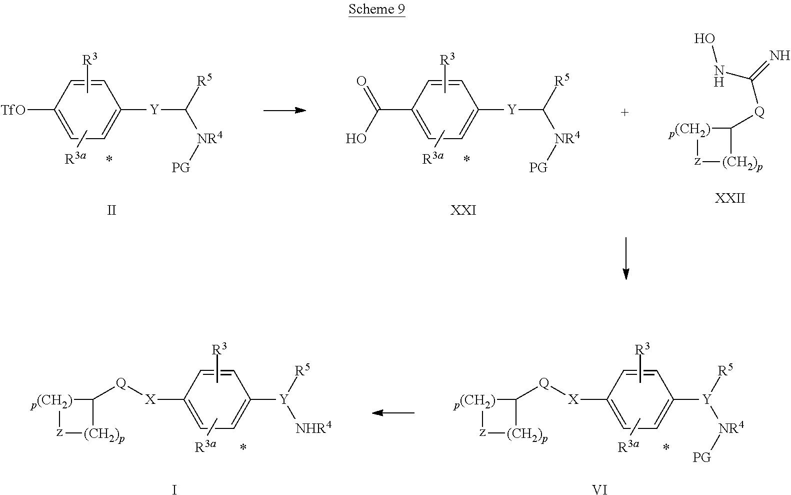

Compounds of formula (I) where p is 2, Q is —O—CR⁸H— and X is thiazol-2-yl can be prepared as outlined in Scheme 10. Amide of formula (XXIII) can be prepared by reaction of nitrile of formula (XVIII) with hydrogen peroxide in a suitable solvent such as water/DMSO. Thioamide of formula (XXIV) can be prepared by reaction of amide of formula (XXIII) under standard conditions, for example using Lawesson's reagent in a suitable solvent such as toluene at reflux. Compounds of formula (VI) can be prepared by reaction of thioamide of formula (XXIV) with chloride of formula (XXV) in the presence of a suitable base, such as K₂CO₃ in a suitable solvent such as acetone. Deprotection of the amine functionality, using standard conditions well known to those with skill in the art, affords compounds of formula (I) as described above.

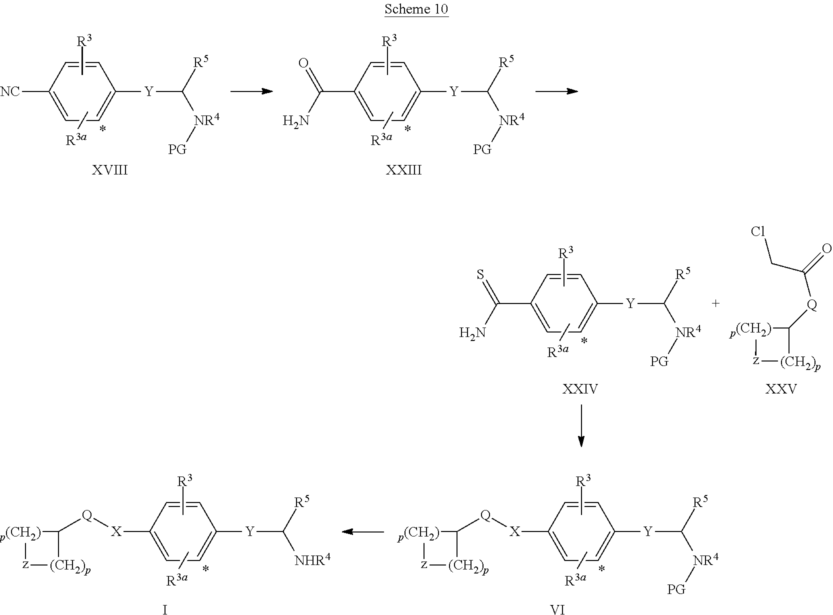

Compounds of formula (I) where p is 2, Q is —O—CR⁸H— and X is thiazol-4-yl can be prepared as outlined in Scheme 11. Ketones of formula (XXVI) can be prepared by reaction of triflate of formula (II) with vinylbutylether in the presence of a suitable catalyst, such as palladium acetate, in a suitable solvent, such as DMF at 80° C. Followed by work up with aqueous HCl solution at room temperature. Bromoketones of formula (XXVII) can be prepared by reaction of ketones of formula (XXVI) with trimethylphenylammonium tribromide in a suitable solvent, such as THF. Compounds of formula (VI) can be prepared by reaction of bromoketones of formula (XXVII) with thioamide of formula (XXVIII) under standard Hantzsch conditions, for example ethanol at room temperature. Deprotection of the amine functionality, using standard conditions well known to those with skill in the art, affords compounds of formula (I) as described above.

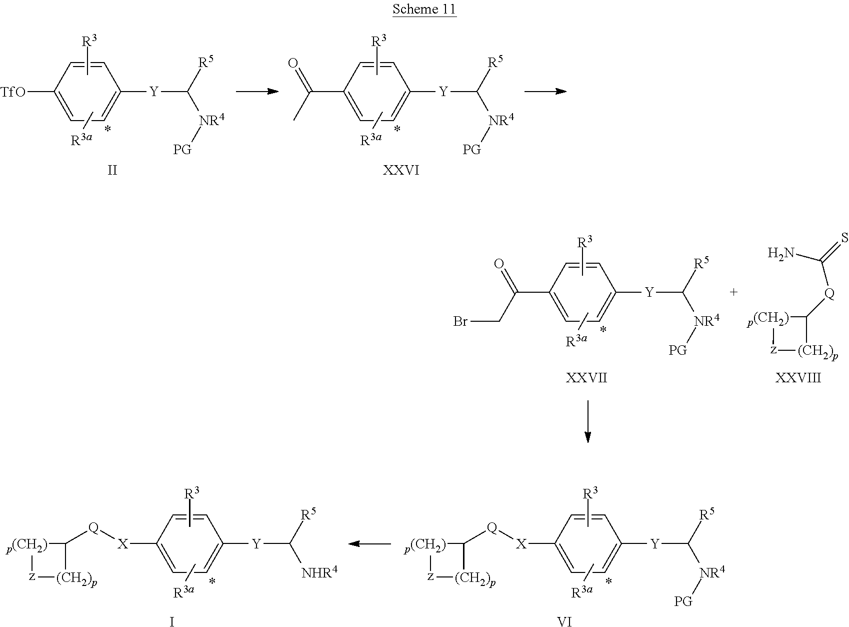

Compounds of formula (XIV) can be prepared as outlined in Scheme 12. Compounds of formula (XIV) can be prepared by reaction of triflate of formula (II) with bis(pinacolato)diboron in the presence of a suitable catalyst, such as [1,1-bis(diphenylphosphino)ferrocene]dichloropalladium in a suitable solvent such as 1,4-dioxane at 110° C.

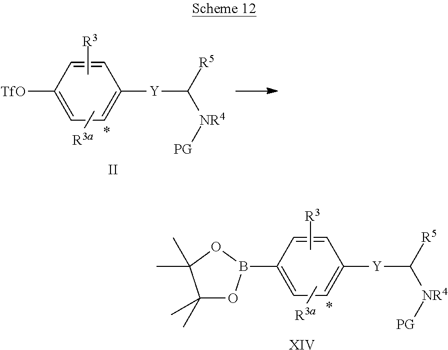

Compounds of formula (II) where R⁵ is benzyl can be prepared as outlined in Scheme 13. Aldehydes of formula (XXX) can be prepared by conversion of phenols of formula (XXIX) under standard conditions, for example, N-phenyltrifluoromethane sulfonimide in a suitable solvent, such as acetonitrile at room temperature. Amine of formula (XXXI) can be prepared by reaction of aldehyde of formula (XXX) with LiHMDS, followed by reaction of the resultant imine with a suitable Grignard reagent. Protection of the resulting amine group with, for example, di-tert-butyldicarbonate, affords compounds of the formula (II).

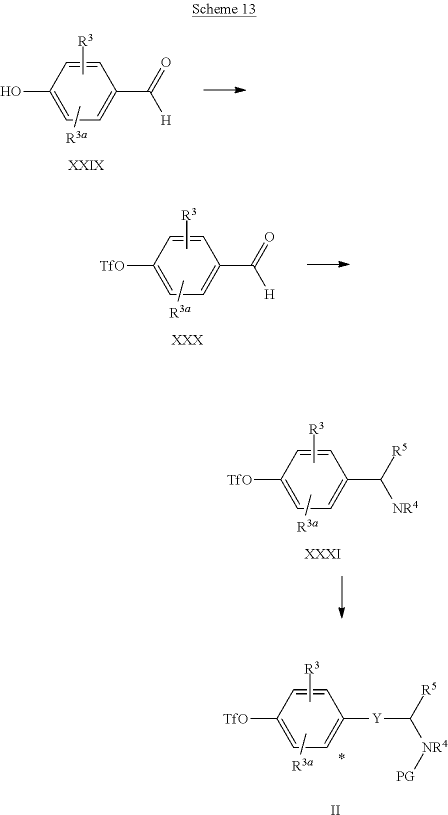

Compounds of formula (II) where R⁵ is amide, W is CH₂ or S, m is 1 and R⁷ is cyano can be prepared as outlined in Scheme 14. Amides of formula (XXXIII) can be prepared by reaction of acids of formula (XXXII) with an appropriate amine under standard amide coupling conditions, for example, HOBT and EDCI, in a suitable solvent, such as DCM. Triflates of formula (XXXIV) can be prepared by conversion of amides of formula (XXXIII) under standard conditions, for example, N-phenyltrifluoromethane sulfonimide in a suitable solvent, such as acetonitrile at room temperature. Compounds of formula (II), as described above, can be prepared by reaction of compounds of formula (XXXIV) under standard dehydrating conditions, such as trifluoroacetic anhydride and pyridine in a suitable solvent, such as THF.

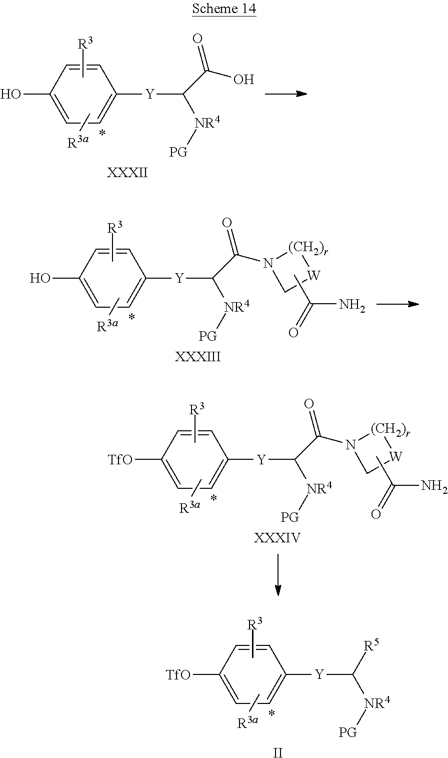

Compounds of formula (II) where R⁵ is amide and R⁷ is not cyano can be prepared as outlined in Scheme 15. Amides of formula (XXXV) can be prepared by reaction of acids of formula (XXXII) with an appropriate amine under standard amide coupling conditions, for example, HOBT and EDCI in a suitable solvent, such as DCM. Triflates of formula (II) can be prepared by conversion of amides of formula (XXXIII) under standard conditions, for example, N-phenyltrifluoromethane sulfonimide in a suitable solvent, such as acetonitrile at room temperature.

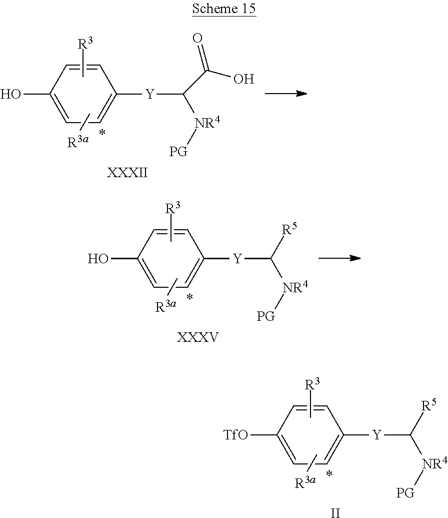

Specifically, chiral compounds of formula (XXXII) where R³ is fluorine, R^{3a} is hydrogen, Y is CHMe and R⁴ is hydrogen can be prepared as outlined in Scheme 16. The compound of formula (XXXVII) can be synthesized by reaction of 4-benzyloxy-2-fluorobenzaldehyde (XXXVI) with methyl(triphenylphosphoranylidene)acetate in a suitable solvent, such as THF, under reflux conditions. Saponification, followed by activation of the resulting carboxylic acid with, for example, pivaloyl chloride, followed by reaction with (R)-(−)-4-phenyl-2-oxazolidinone which has been deprotonated with a suitable base, such as n-butyllithium, affords the compound of formula (XXXIX). Reaction with dimethyl sufide, methyl magnesium bromide and copper (I) bromide-dimethyl sulfide in a suitable solvent, such as THF, yields the compound of formula (XL). Subsequent reaction with dibutylborontriflate and N-bromosuccinimide, followed by reaction with N,N,N′,N′-tetramethylguanidinium azide, affords the compound of formula (XLII). Removal of the phenyloxazolidin-2-one group, with hydrogen peroxide and sodium hydroxide, gives the compound of formula (XLIII). Reduction, under standard conditions, followed by protection of the resulting amine group with, for example, di-tert-butyldicarbonate, affords compounds of the formula (XXXII) as described above.

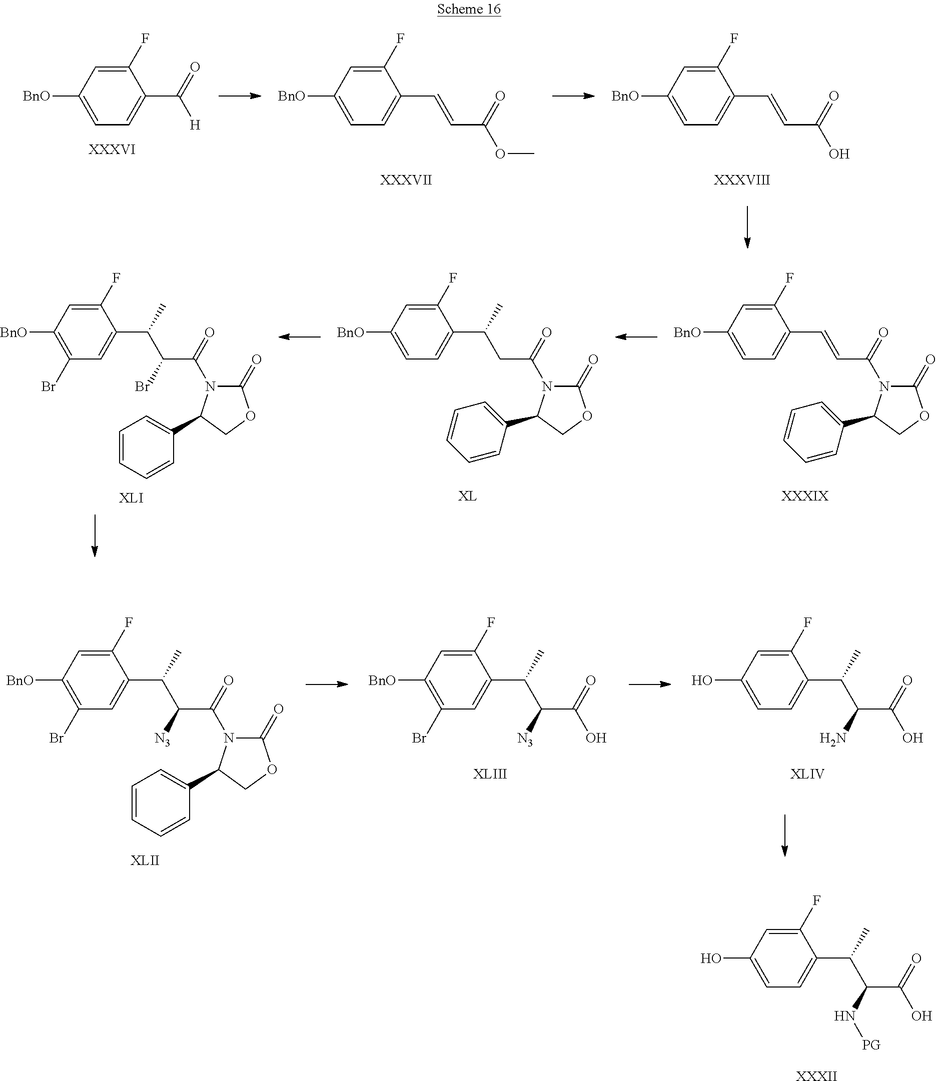

Specifically, compounds of formula (XXXII) where R³ is fluorine, R^{3a} is hydrogen, Y is CH₂ and R⁴ is hydrogen can be prepared as outlined in Scheme 17. Compounds of formula (XLVI) can be prepared by reaction of 2-fluoro-4-methoxybenzaldehyde (XLV) with sodium acetate and acetylaminoacetic acid at 120° C. in acetic anhydride. Reduction of the resulting alkenoic acid (XLVI), under standard conditions, affords a racemic compound of formula (XLVII). Reduction of the alkenoic acid (XLVI) with a chiral catalyst, such as [Rh(cod)(PP)]OTf and (S,S)-Et-Duphos, affords a compound of formula (XLVII) in high enantiomeric excess. Removal of the acetyl group, under standard acidic conditions, followed by protection of the amine group with, for example, di-tert-butyldicarbonate yields compounds of formula (XXXII) as described above.

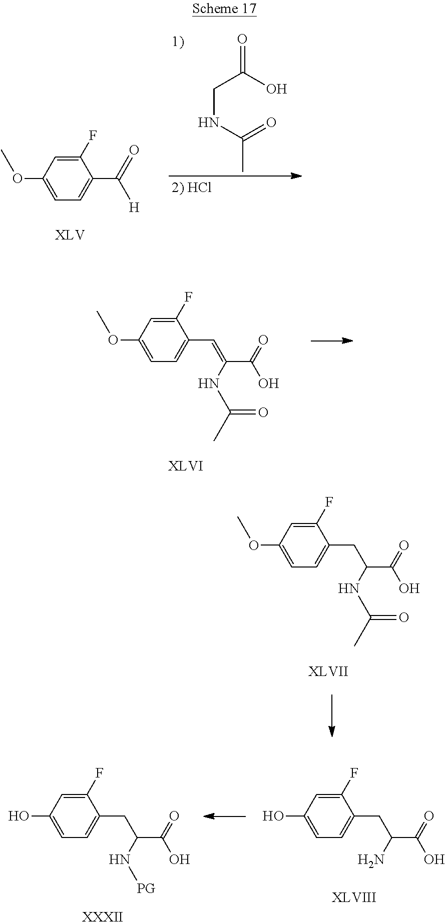

Compounds of formula (XXV) where p is 2 and Q is —O—CR⁸H— can be prepared as outlined in Scheme 18. Alcohols of formula (XVII) can be treated with 1,3-dichloroacetone in the presence of a suitable base, such as K₂CO₃, in a suitable solvent such as DMF to give compounds of formula (XXV) as described above.

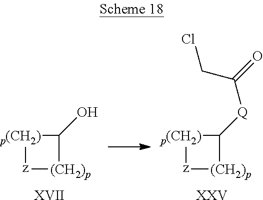

Compounds of formula (XXVIII) where p is 2 and Q is —O—CR⁸H— can be prepared as outlined in Scheme 19. Amides of formula (XLIX) can be prepared by reaction of acids of formula (XX) with an appropriate amine under standard amide coupling conditions, for example, HOBT and EDCI, in a suitable solvent, such as DCM. Thioamide of formula (XXVIII) can be prepared by reaction of amide of formula (XLIX) under standard conditions, for example using Lawesson's reagent in a suitable solvent such as toluene at reflux.

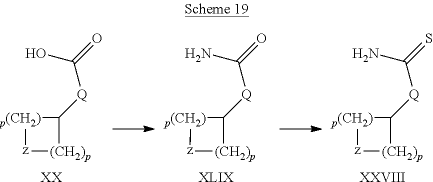

Other compounds of formula (I) may be prepared by methods analogous to those described above or by methods known per se. Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

The processes for the production of the compounds of formula (I) and intermediates thereto as described above are also included as further aspects of the present invention.

Any novel intermediates as defined in the Schemes above or in the Examples, are also included within the scope of the invention. Therefore according to a further aspect of the invention there is provided a compound of any one of formulae II, IV, VI, XIV, XVIII, XIX, XXI, XXIII, XXIV, XXIV, XXVI, XXVII, XXXI, XXXIV as defined above. The preferred groups for variables recited above in relation to the compounds of formula (I) also apply to the intermediate compounds.

As indicated above the compounds of the invention are useful as GPR119 agonists, e.g. for the treatment and/or prophylaxis of diabetes. For such use the compounds of the invention will generally be administered in the form of a pharmaceutical composition.

The compounds of the invention may also be useful as dual GPR119 agonists/DPP-IV inhibitors, e.g. for the treatment and/or prophylaxis of diabetes. For such use the compounds of the invention will generally be administered in the form of a pharmaceutical composition.

The invention also provides a compound of the invention, or a pharmaceutically acceptable salt thereof, for use as a pharmaceutical.

The invention also provides a pharmaceutical composition comprising a compound of the invention, in combination with a pharmaceutically acceptable carrier.

Preferably the composition is comprised of a pharmaceutically acceptable carrier and a non-toxic therapeutically effective amount of a compound of the invention, or a pharmaceutically acceptable salt thereof.

Moreover, the invention also provides a pharmaceutical composition for the treatment of disease by modulating GPR119 and optionally DPP-IV, resulting in the prophylactic or therapeutic treatment of diabetes, comprising a pharmaceutically acceptable carrier and a non-toxic therapeutically effective amount of compound of the invention, or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of the invention, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of the invention, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of the invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of the invention may be used in the treatment of diseases or conditions in which GPR119 and optionally DPP-IV play a role.

Thus the invention also provides a method for the treatment of a disease or condition in which GPR119 and optionally DPP-IV play a role comprising a step of administering to a subject in need thereof an effective amount of a compound of the invention, or a pharmaceutically acceptable salt thereof. Such diseases or conditions diabetes, obesity, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The invention also provides a method for the treatment of type II diabetes, comprising a step of administering to a patient in need thereof an effective amount of a compound of the invention, or a pharmaceutically acceptable salt thereof.

The invention also provides a method for the treatment of obesity, metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of the invention, or a pharmaceutically acceptable salt thereof.

The invention also provides a compound of the invention, or a pharmaceutically acceptable salt thereof, for use in the treatment of a condition as defined above.

The invention also provides the use of a compound of the invention, or a pharmaceutically acceptable salt thereof, in the manufacture of a medicament for the treatment of a condition as defined above.

In the methods of the invention the term “treatment” includes both therapeutic and prophylactic treatment.

The compounds of the invention may exhibit advantageous properties compared to known compounds or combination therapies for the treatment of diabetes.

The compounds of the invention, or pharmaceutically acceptable salts thereof, may be administered alone or in combination with one or more other therapeutically active compounds. The other therapeutically active compounds may be for the treatment of the same disease or condition as the compounds of the invention or a different disease or condition. The therapeutically active compounds may be administered simultaneously, sequentially or separately.

The compounds of the invention may be administered with other active compounds for the treatment of obesity and/or diabetes, for example insulin and insulin analogs, gastric lipase inhibitors, pancreatic lipase inhibitors, sulfonyl ureas and analogs, biguanides e.g. metformin, α2 agonists, glitazones, PPAR-γ agonists, mixed PPAR-α/γ agonists, RXR agonists, fatty acid oxidation inhibitors, α-glucosidase inhibitors, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of the invention, or a pharmaceutically acceptable salt thereof, and at least one other agent, for example another agent for the treatment of diabetes or obesity, represents a further aspect of the invention.

The present invention also provides a method for the treatment of diabetes in a mammal, such as a human, which method comprises administering an effective amount of a compound of the invention, or a pharmaceutically acceptable salt thereof, and another agent, for example another agent for the treatment of diabetes or obesity, to a mammal in need thereof.

The invention also provides the use of a compound of the invention, or a pharmaceutically acceptable salt thereof, and another agent for the treatment of diabetes.

The invention also provides the use of a compound of the invention, or a pharmaceutically acceptable salt thereof, in the manufacture of a medicament for use in combination with another agent, for the treatment of diabetes.

The compound of the invention, or a pharmaceutically acceptable salt thereof, and the other agent(s) may be co-administered or administered sequentially or separately.

Co-administration includes administration of a formulation which includes both the compound of the invention, or a pharmaceutically acceptable salt thereof, and the other agent(s), or the simultaneous or separate administration of different formulations of each agent. Where the pharmacological profiles of the compound of the invention, or a pharmaceutically acceptable salt thereof, and the other agent(s) allow it, coadministration of the two agents may be preferred.

The invention also provides the use of a compound of the invention, or a pharmaceutically acceptable salt thereof, and another agent in the manufacture of a medicament for the treatment of diabetes.

The invention also provides a pharmaceutical composition comprising a compound of the invention, or a pharmaceutically acceptable salt thereof, and another antidiabetic agent, and a pharmaceutically acceptable carrier. The invention also encompasses the use of such compositions in the methods described above.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and Methods

Column chromatography was carried out on SiO₂ (40-63 mesh) unless specified otherwise. LCMS data were obtained as follows: Atlantis 3μ C₁₈ column (3.0×20.0 mm, flow rate=0.85 mL/min) eluting with a H₂O—CH₃CN solution containing 0.1% HCO₂H over 6 min with UV detection at 220 nm. Gradient information: 0.0-0.3 min 100% H₂O; 0.3-4.25 min: Ramp up to 10% H₂O—90% CH₃CN; 4.25-4.4 min: Ramp up to 100% CH₃CN; 4.4-4.9 min: Hold at 100% CH₃CN; 4.9-6.0 min: Return to 100% H₂O. The mass spectra were obtained using an electrospray ionisation source in either the positive (ES⁺) or negative (ES⁻) ion modes.

LCMS data (method 2) were obtained as follows: Chromolith SpeedROD column (4.6×50.0 monolith, flow rate=3.0 mL/min) eluting with a H₂O—CH₃CN solution containing 0.1% TFA over 3 min with UV detection at 220 nm. Gradient information: 0-2 min: 99% H₂O 1% MeCN to 100% MeCN; 2-3 min: Hold at 100% CH₃CN. The mass spectra were obtained using an electrospray ionisation source in the positive (ES⁺) mode.

Chiral-HPLC was performed on a Daicel chiralpak IA 250×20 mm, 5 μM column.

Abbreviations and acronyms: Ac: Acetyl; AcOH: Acetic acid; ADDP: Azodicarboxylic dipiperidide; Boc: tert-butyloxycarbonyl; t-Bu: tent-Butyl; DBU: 1,8-Diazabicyclo[5.4.0]undec-7-ene; DCE: 1,2-Dichloroethane; DCM: Dichloromethane; DIPEA: N,N-Diisopropylethylamine; DMF: Dimethylformamide; EDCI: 1-(3-Dimethylaminopropyl)-3-ethylcarbodiimide hydrochloride; EtOH: Ethanol; Et: Ethyl; EtOAc: Ethyl acetate; eq: Equivalents; h: hour(s); min: minute/s; HATU: O-(7-Azabenzotriazol-1-yl)-N,N,N′,N′-tetramethyluronium hexafluorophosphate; HCl: Hydrochloric acid; HPLC: High performance liquid chromatography; H₂O: Water; HOB t: 1-Hydroxybenzotriazole; IH: Isohexane; LiHMDS: Lithium bis(trimethylsilyl)amide; MeOH: Methanol; Me: Methyl; MeCN: Acetonitrile; MP: Macroporous Polystyrene; MgSO₄: Magnesium sulphate; MTBE: Methyl tert-butyl ether; Na₂CO₃: Sodium carbonate; Na₂SO₃: Sodium sulfite; Na₂SO₄: Sodium sulphate; NaHCO₃: Sodium hydrogen carbonate; NaOH: Sodium hydroxide; NH₄Cl: Ammonium chloride; PBu₃: tri-tert-butyl phosphine; PE-AX column: silica based quaternary amine column; RP: Reverse Phase; RT: Retention time; r.t.: Room temperature; sat: Saturated; SiO₂: Silica; TBAF: Tetra-butyl ammonium fluoride; THF: Tetrahydrofuran; TFA: Trifluoroacetic acid; TFAA: Trifluoroacetic anhydride; TMS: Trimethylsilyl.

The syntheses of the following compounds have been described elsewhere: [1-(3-isopropyl-[1,2,4]oxadiazol-5-yl)piperidin-4-yl]methanol: Jing et. al., WO2008/070692; (4-hydroxycyclohexyl)methylcarbamic acid isopropyl ester: Ackermann et. al., WO02/014267; 4-carboxymethoxypiperidine-1-carboxylic acid isopropyl ester: Fyfe et. al., WO2007/116229. All other compounds were available from commercial sources.

### Preparation 1

### 4-Hydroxymethyl piperidine-1-carboxylic acid isopropyl ester

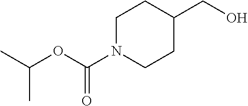

To a solution of 4-piperidine methanol (12 g, 104.12 mmol) in DCM (200 mL) was added DIPEA (23.6 mL, 135.42 mmol) and the reaction was cooled to 0° C. A solution of isopropylchloroformate (120 mL, 119.79 mmol) in toluene (120 mL) was added dropwise, over 1.5 h, then the reaction was brought to r.t. and stirred for a further 2.5 h. The reaction mixture was partitioned with 1M HCl solution (200 mL) then the organic layer was removed and washed with 1M HCl solution (200 mL), brine (200 mL) and dried (MgSO₄). Removal of the solvent in vacuo afforded the title compound: ¹H NMR δ_{H} (400 MHz, CDCl₃): 4.96-4.86 (m, 1H), 4.09-4.25 (m, 2H), 3.51 (d, J=6.2 Hz, 2H), 2.80-2.68 (m, 2H), 1.78-1.62 (m, 3H), 1.49-1.41 (m, 1H), 1.29-1.09 (m, 8H).

### Preparation 2

### 4-Hydroxypiperidine-1-carboxylic acid isopropyl ester

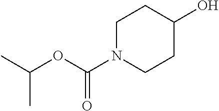

The title compound was prepared from 4-hydroxypiperidine employing a procedure similar to that outlined in Preparation 1: ¹H NMR δ_{H} (400 MHz, CDCl₃): 4.96-4.87 (m, 1H), 3.94-3.82 (m, 3H), 3.13-3.04 (m, 2H), 1.92-1.82 (m, 2H), 1.57-1.54 (m, 1H), 1.54-1.42 (m, 2H), 1.26-1.22 (m, 6H).

### Preparation 3

### 4-(4-Bromophenoxy)piperidine-1-carboxylic acid isopropyl ester

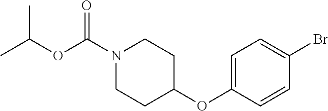

4-Hydroxypiperidine-1-carboxylic acid isopropyl ester (Preparation 2, 4.68 g, 25 mmol), 4-bromophenol (5.19 g, 30 mmol) and triphenylphosphine (7.87 g, 25 mmol) were dissolved in DCM (125 mL) and di tert-butylazodicarboxylate (6.90 g, 30 mmol) was added, portionwise, over 20 min. The reaction was stirred at r.t. for 72 h and then diluted with DCM (150 mL). The organic solution was washed with 2M NaOH solution (2×200 mL), brine (200 mL) then dried (MgSO₄). Removal of the solvent in vacuo followed by purification by column chromatography (SiO₂, 1H:EtOAc, 90:10, 80:20, 70:30) afforded the title compound: RT=4.09 min; m/z (ES⁺)=342.1, 344.0 [M+H]⁺.

### Preparation 4

### 4-(5-Bromopyridin-2-yloxy)piperidine-1-carboxylic acid isopropyl ester

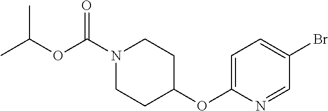

A dry solution of 4-hydroxypiperidine-1-carboxylic acid isopropyl ester (Preparation 2, 10 g, 53 mmol) in DMF, under argon, was cooled to 0° C. Sodium hydride (60% in mineral oil, 2.54 g, 64 mmol) was added in one portion. The reaction was allowed to reach r.t. before stirring for a further 45 min. 5-Bromo-2-chloropyridine (12.32 g, 64 mmol) was added and the reaction was heated to 60° C. for 40 h. The reaction mixture was allowed to cool to r.t. then EtOAc was added. The organic solution was washed with brine, dried (MgSO₄) and solvent was removed in vacuo. The crude material was triturated from iso-hexane (2×6 mL) then diethyl ether to afford the title compound: RT=3.98 min; m/z (ES⁺)=343.0, 345.0 [M+H]⁺.

### Preparation 5

### 4-(5-Bromopyridin-2-yloxymethyl)piperidine-1-carboxylic acid isopropyl ester

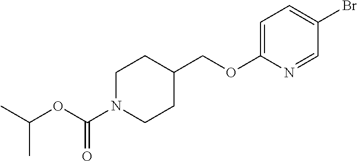

The title compound was prepared from 4-hydroxymethyl piperidine-1-carboxylic acid isopropyl ester (Preparation 1, 2.5 g, 12.42 mmol) and 5-bromo-2-chloropyridine (2.8 g, 14.4 mmol) employing a procedure similar to that outlined in Preparation 4: RT=4.20 min; m/z (ES⁺)=357.1, 359.1 [M+H]⁺.

### Preparation 6

### 4-Methanesulfonyloxymethylpiperidine-1-carboxylic acid isopropyl ester

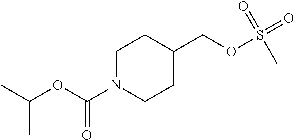

To a dry solution of 4-hydroxymethylpiperidine-1-carboxylic acid isopropyl ester (Preparation 1, 2.5 g, 12.42 mmol) in DCM (30 mL) under argon was added triethylamine (2.08 mL, 14.9 mmol) and the mixture was cooled to 0° C. Methanesulfonyl chloride (1.06 mL, 13.66 mmol) was added, dropwise, over 4 min then the reaction was stirred at 0° C. for 30 min. The mixture was diluted with DCM (50 mL) and the organic layer was washed with water (2×50 mL), 0.5M HCl solution (2×50 mL), brine (50 mL) then dried (MgSO₄). Removal of the solvent in vacuo afforded the title compound: ¹H NMR δ_{H}(400 MHz, DMSO-d₆): 4.95-4.85 (m, 1H), 4.25-4.18 (m, 2H), 4.17-4.07 (m, 2H), 3.31 (s, 3H), 2.98-2.79 (m, 2H), 2.08-1.96 (m, 1H), 1.85-1.75 (m, 2H), 1.35-1.17 (m, 8H).

### Preparation 7

### 4-(4-Bromophenoxymethyl)piperidine-1-carboxylic acid isopropyl ester

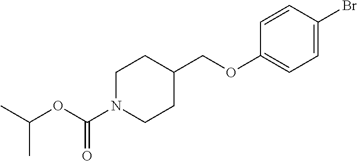

To a solution of 4-methanesulfonyloxymethylpiperidine-1-carboxylic acid isopropyl ester (Preparation 6, 3.4 g, 12.17 mmol) and 4-bromophenol (2.32 g, 13.39 mmol) in DMF (70 mL) under argon was added potassium carbonate (3.36 g, 24.34 mmol) and the reaction was heated to 90° C. for 16 h. The reaction solvent was removed in vacuo and crude residue was dissolved in EtOAc (200 mL) before being washed with water (3×100 mL). The aqueous layers were combined and extracted with EtOAc (50 mL). The organic fractions were combined and washed with sat. NaHCO₃ solution (2×150 mL), brine (150 mL), then dried (MgSO₄). Removal of the solvent in vacuo afforded the title compound: RT=4.36 min; m/z (ES⁺)=356.2, 358.2 [M+H]⁺.

### Preparation 8

### 1-Piperidin-4-yl ethanol

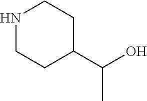

To a solution of α-methyl-4-pyridine methanol (3.7 g, 30 mmol) in EtOH (100 mL) was added AcOH (1.9 mL, 33 mmol) and platinum oxide (0.5 g, 2.2 mmol) and the resulting mixture was allowed to stir under an atmosphere of hydrogen at r.t. for 16 h. The mixture was filtered and the filtrate was concentrated in vacuo. The residue was dissolved in MeOH, to which was added a solution of NaOH (1.6 g, 40 mmol) and water (1.6 mL) in MeOH. The reaction was stirred for 30 min before removing the solvent in vacuo, and the resulting residue was suspended in diethyl ether for 30 min. The mixture was filtered and the filtrate was concentrated in vacuo to afford the title compound: ¹H NMR δ_{H} (400 MHz, CDCl₃): 3.63-3.55 (m, 1H), 3.39-3.31 (m, 2H), 2.7-2.6 (m, 2H), 2.01-1.92 (m, 2H), 1.76-1.69 (m, 1H), 1.67-1.54 (m, 2H), 1.51-1.42 (m, 1H), 1.1-1.14 (m, 3H).

### Preparation 9

### 4-(1-Hydroxyethyl)piperidine-1-carboxylic acid isopropyl ester

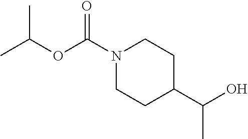

The title compound was prepared from 1-piperidin-4-yl ethanol (Preparation 8, 2.7 g, 20.93 mmol) employing the procedure outlined in Preparation 1: ¹H NMR δ_{H} (400 MHz, CDCl₃): 4.97-4.87 (m, 1H), 4.28-4.14 (m, 2H), 3.66-3.55 (m, 1H), 2.77-2.63 (m, 2H), 1.88-1.81 (m, 1H), 1.67-1.59 (m, 1H), 1.48-1.38 (m, 1H), 1.26-1.16 (m, 11H).

### Preparation 10

### 4-[1-(5-Bromopyridin-2-yloxy)ethyl]piperidine-1-carboxylic acid isopropyl ester

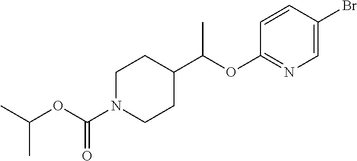

The title compound was prepared by reacting 4-(1-hydroxyethyl)piperidine-1-carboxylic acid isopropyl ester (Preparation 9, 7.4 g, 57.36 mmol) with 2-chloro-5-bromopyridine (13.2 g, 68.8 mmol) employing a procedure similar to that outlined in Preparation 4: RT=4.34 min; m/z (ES⁺)=371.2, 373.2 [M+H]⁺.

### Preparation 11

### 4-(1-Methanesulfonyloxyethyl)piperidine-1-carboxylic acid isopropyl ester

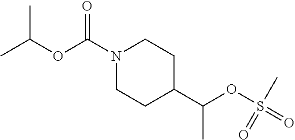

The title compound was prepared from 4-(1-hydroxyethyl)piperidine-1-carboxylic acid isopropyl ester (Preparation 9, 4.3 g, 20 mmol) employing a procedure similar to that outlined in Preparation 6: ¹H NMR δ_{H}(400 MHz, CDCl₃): 4.98-4.86 (m, 1H), 4.69-4.61 (m, 1H), 4.31-4.17 (m, 2H), 3.01 (s, 3H), 2.76-2.66 (m, 2H), 1.84-1.63 (m, 3H), 1.44-1.38 (m, 3H), 1.35-1.22 (m, 8H).

### Preparation 12

### 4-[1-(4-Bromophenoxy)ethyl]piperidine-1-carboxylic acid isopropyl ester

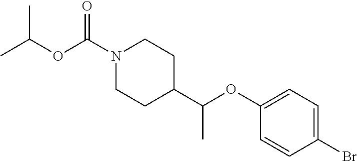

4-(1-Methanesulfonyloxyethyl)piperidine-1-carboxylic acid isopropyl ester (Preparation 11, 100 mg, 0.36 mmol) was reacted with 4-bromophenol (69 mg, 0.40 mmol) employing the conditions outlined in Preparation 4. Work-up involved partitioning the reaction mixture between EtOAc and water. The organic phase was separated and washed with 1M NaOH solution, water, brine, and dried (MgSO₄). Removal of the solvent in vacuo followed by purification by column chromatography (SiO₂, 1H:EtOAc, 4:1) afforded the title compound: RT=4.5 min; m/z (ES⁺)=370.2, 372.1 [M+H]⁺.

### Preparation 13

### 4-(4-Bromopyridin-2-yloxymethyl)piperidine-1-carboxylic acid isopropyl ester

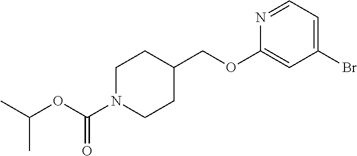

To a solution of 4-hydroxymethyl piperidine-1-carboxylic acid isopropyl ester (Preparation 1, 1.1 g, 5.47 mmol) in THF (15 mL) was added sodium hydride (60% in mineral oil, 218 mg, 5.47 mmol) and the reaction stirred at r.t. for 1 h. 4-Bromo-2-chloropyridine (0.62 mL, 5.47 mmol) was added and the reaction was heated to 90° C. for 4 h before being quenched with water (10 mL). The organics were extracted into EtOAc (3×15 mL), dried (MgSO₄), and solvent was removed in vacuo. Purification by column chromatography (SiO₂, IH:EtOAc, 9:1) afforded the title compound: RT=4.16 min; m/z (ES⁺)=357.1, 359.1 [M+H]⁺.

### Preparation 14

### Azetidin-3-ol

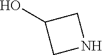

To a solution of 1-benzhydrylazetidin-3-ol (30.5 g, 130 mmol) in EtOH (500 mL) was added a pre-mixed solution of triethylamine (55 mL, 390 mmol) and formic acid (15 mL, 390 mol) in ethanol (100 mL). Palladium on carbon (2.40 g) was added and the mixture heated to reflux for 3 h. The mixture was cooled to r.t. and filtered through celite to afford the title compound as a solution in ethanol.

### Preparation 15

### 1-(4-Isopropylbenzyl)azetidin-3-ol

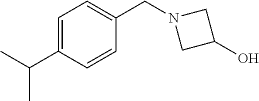

To a solution of azetidin-3-ol (Preparation 14, 6.84 mmol) and 4-isopropylbenzaldehyde (8.21 mmol) in ethanol (45 mL) was added acetic acid (0.5 mL). After stirring for 1 h, sodium triacetoxyborohydride (8.21 mmol) was added, and stiffing continued for 72 h. Aqueous hydrochloric acid (1M, 30 mL) was added and the mixture concentrated to remove ethanol. The mixture was extracted with diethyl ether (×2), and the remaining aqueous mixture basified by addition of 2M NaOH solution. The solution was then extracted with DCM (×3). The combined DCM extracts were dried (MgSO₄) and concentrated to afford the title compound; RT=2.00 min; m/z (ES⁺)=206.1 [M+H]⁺.

### Preparation 16

### 5-Bromo-2-[1-(4-isopropyl benzyl)azetidin-3-yloxy]pyridine

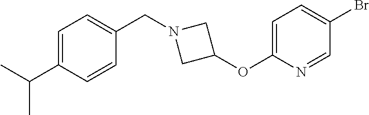

A solution of 1-(4-isopropylbenzyl)azetidin-3-ol (Preparation 15, 500 mg, 2.44 mmol) in DMF (10 mL) under argon was cooled to 0° C. Sodium hydride (60% in mineral oil, 120 mg, 2.92 mmol) was added and the reaction was allowed to reach r.t. 5-Bromo-2-chloropyridine (562.4 mg, 2.92 mmol) was added and the reaction heated to 60° C. for 16 h. Solvent was removed in vacuo and purification by column chromatography (SiO₂, DCM:MeOH, 100:0, 90:10) afforded the title compound: RT=2.98 min; m/z (ES⁺)=361.2, 363.2 [M+H]⁺.

### Preparation 17

### 4-[5-(4,4,5,5-Tetramethyl-[1,3,2]dioxaborolan-2-yl)pyridin-2-yloxy]piperidine-1-carboxylic acid isopropyl ester

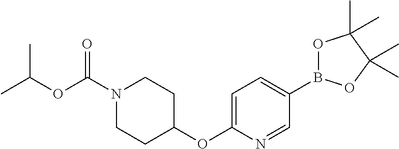

To a solution of 4-(5-bromopyridin-2-yloxy)piperidine-1-carboxylic acid isopropyl ester (Preparation 4, 5.0 g, 14.6 mmol) in dioxane (100 mL) was added potassium acetate (4.3 g, 43.7 mmol), [1,1-bis(diphenylphosphino)ferrocene]dichloropalladium (1.2 g, 1.5 mmol) and bis(pinacolato)diboron (4.42 g, 17.4 mmol). Argon was bubbled through the reaction mixture for 15 min. The reaction was heated to 110° C. for 16 h, and then allowed to stir for a further 72 h at r.t. Removal of the solvent in vacuo followed by purification by column chromatography (SiO₂, DCM) afforded the title compound: RT=4.00 min; m/z (ES⁺)=391.2 [M+H]⁺.

The following compounds were prepared by reacting the appropriate aryl or heteroaryl bromide with bis(pinacolato)diboron employing a procedure similar to that outlined in Preparation 17:

| Prep | | | |
|---|---|---|---|
| No. | Structure | Name | Spectra |
| 18 | 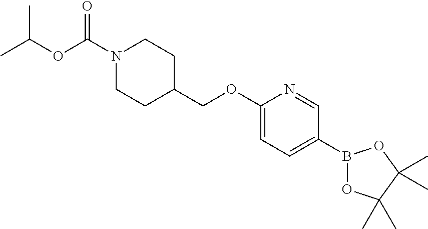 | 4-[5-(4,4,5,5-Tetramethyl- [1,3,2]dioxaborolan-2- yl)pyridin-2-yloxymethyl]- piperidine-1-carboxylic acid isopropyl ester | ¹H NMR δ_{H} (400 MHz, CDCl₃): 8.55- 8.48 (m, 1H), 7.96-7.89 (m, 1H), 6.73- 6.66 (m, 1H), 5.00-4.86 (m, 1H), 4.24- 4.14 (m, 4H), 2.83-2.71 (m, 2H), 2.03- 1.92 (m, 1H), 1.86-1.75 (m, 2H), 1.30- 1.19 (m, 20H) |
| 19 | 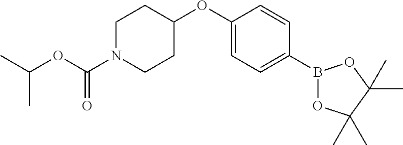 | 4-[4-(4,4,5,5-Tetramethyl- [1,3,2]dioxaborolan-2- yl)phenoxy]piperidine-1- carboxylic acid isopropyl ester | RT = 4.24 min; m/z (ES⁺) = 390.4 [M + H]⁺ |
| 20 | 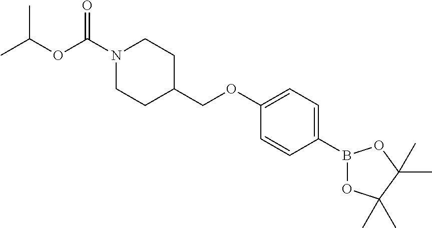 | 4-[4-(4,4,5,5-Tetramethyl- [1,3,2]dioxaborolan-2- yl)phenoxymethyl]piperidine- 1-carboxylic acid isopropyl ester | RT = 4.52 min; m/z (ES⁺) = 404.4 [M + H]⁺ |
| 21 | 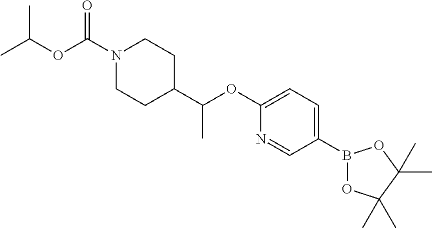 | 4-{1-[5-(4,4,5,5-Tetramethyl- [1,3,2]dioxaborolan-2- yl)pyridin-2-yloxy]ethyl}- piperidine-1-carboxylic acid isopropyl ester | RT = 4.55 min; m/z (ES⁺) = 419.4 [M + H]⁺ |
| 22 | 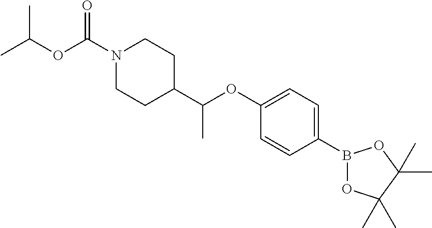 | 4-{1-[4-(4,4,5,5-Tetramethyl- [1,3,2]dioxaborolan-2- yl)phenoxyethyl}piperidine- 1-carboxylic acid isopropyl ester | RT = 4.63 min; m/z (ES⁺) = 418.4 [M + H]⁺ |
| 23 | 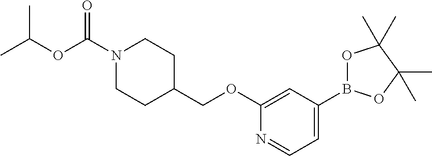 | 4-[4-(4,4,5,5-Tetramethyl- [1,3,2]dioxaborolan-2- yl)pyridin-2-yloxymethyl]- piperidine-1-carboxylic acid isopropyl ester | ¹H NMR δ_{H} (400 MHz, CDCl₃): 8.20- 8.11 (m, 1H), 7.20-7.16 (m, 1H), 7.15- 7.10 (m, 1H), 4.97-4.89 (m, 1H), 4.20 (m, 2H), 4.19 (d, 2H), 2.80- 2.71 (m, 2H), 1.99-1.94 (m, 1H), 1.84- 1.78 (m, 2H), 1.4 (s, 12H), 1.3 (m, 2H) 1.25 (d, 6H) |

### Preparation 24

### 4-(3-Bromophenoxymethyl)piperidine-1-carboxylic acid tert-butyl ester

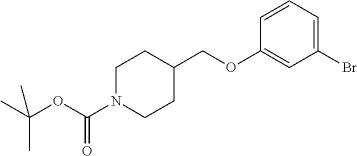

The title compound was prepared by reacting 4-hydroxymethylpiperidine-1-carboxylic acid tert-butyl ester (1.075 g, 5 mmol) with 3-bromophenol (1.026 g, 6 mmol) employing the procedure outlined in Preparation 3: RT=4.50 min; m/z (ES⁺)=370.2, 372.2 [M+H]⁺.

### Preparation 25

### 4-(3-Bromophenoxymethyl)piperidine-1-carboxylic acid isopropyl ester

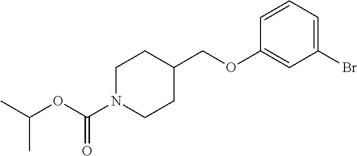

To a solution of 4-(3-bromophenoxymethyl)piperidine-1-carboxylic acid tert-butyl ester (Preparation 24, 1.5 g, 4.05 mmol) in DCM (8 mL) was added TFA (2 mL) and the reaction stirred at r.t. for 15 min. The mixture was diluted with DCM (150 mL) and quenched with sat. NaHCO₃ solution (150 mL). Organics were separated and washed with brine then dried (MgSO₄) and the solvent removed in vacuo. The residue was re-dissolved in DCM (20 mL) and DIPEA (0.8 mL) was added before cooling the reaction to 0° C. Isopropyl chloroformate (1M in toluene, 4.86 mL, 4.86 mmol) was added via syringe and the reaction was allowed to warm to r.t. before stirring for a further 16 h. The mixture was diluted with EtOAc (150 mL) and the resulting solution was washed with 1M citric acid solution then brine, and dried (MgSO₄). Removal of the solvent in vacuo afforded the title compound: RT=4.34 min; m/z (ES⁺)=355.2, 357.2 [M+H]⁺.

### Preparation 26

### 4-[3-(4,4,5,5-Tetramethyl-[1,3,2]dioxaborolan-2-yl)phenoxymethyl]piperidine-1-carboxylic acid isopropyl ester

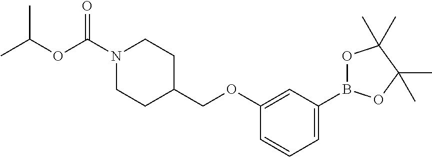

To a solution of 4-(3-bromophenoxymethyl)piperidine-1-carboxylic acid isopropyl ester in dioxane (Preparation 25, 430 mg, 1.21 mmol) was added potassium acetate (355 mg, 3.62 mmol) and bis(pinacolato)diboron (369 mg, 1.45 mmol) and the mixture purged with argon. [1,1-bis(diphenylphosphino)ferrocene]dichloropalladium (98 mg, 0.12 mmol) was added and the reaction was purged for a further 5 min before heating to 100° C. for 20 h. After this time celite was added and the mixture was filtered, washing with EtOAc. The resulting organic filtrate was washed with sat. NaHCO₃ solution (150 mL) then brine (150 mL) and dried (MgSO₄). Removal of the solvent in vacuo followed by purification by column chromatography (SiO₂, 1H:EtOAc, 7:3) afforded the title compound: RT=4.44 min; m/z (ES⁺)=404.3 [M+H]⁺.

### Preparation 27

### (S)-2-Amino-3-(2-fluoro-4-hydroxyphenyl)propionic acid hydrobromide

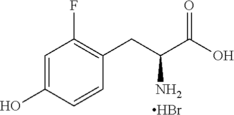

Acetic anhydride (540 g, 5.30 mol) was added under stirring to a mixture of 2-fluoro-4-methoxybenzaldehyde (240 g, 1.56 mol), N-acetylglycine (219 g, 1.87 mol) and sodium acetate (128 g, 1.56 mol) at ambient temperature. The suspension was heated to 100° C. for 18 h. The solution was cooled to ambient temperature and the residue was alternately extracted with DCM (5×500 mL) and water (5×200 mL). The remaining crystalline solid was dried to yield 4-[1-(2-fluoro-4-methoxyphenyl)meth-(E)-ylidene]-2-methyl-4H-oxazol-5-one. The DCM extracts were combined, dried (Na₂SO₄), filtered and concentrated in vacuo. The residue was recrystallized twice from EtOH to provide further 4-[1-(2-fluoro-4-methoxyphenyl)meth-(E)-ylidene]-2-methyl-4H-oxazol-5-one: RT=3.00 min (LCMS method 2).

To 4-[1-(2-fluoro-4-methoxyphenyl)meth-(E)-ylidene]-2-methyl-4H-oxazol-5-one (150.9 g, 0.642 mol) in dioxane (700 mL) was added 1M HCl solution (1000 mL) and the mixture was heated under reflux conditions for 90 min. The dioxane was widely removed by evaporation and the aqueous layer was extracted with EtOAc (×2) and DCM (×2). The combined organic layers were evaporated and the residue was recrystallized from EtOAc/heptane to afford (Z)-2-acetylamino-3-(2-fluoro-4-methoxyphenyl)acrylic acid: RT=1.73 min (LCMS method 2). (Z)-2-Acetylamino-3-(2-fluoro-4-methoxyphenyl)acrylic acid (35.0 g, 138 mmol) was dissolved in MeOH (670 mL) and hydrogenated in an autoclave for 96 h with a pressure of 8 bar at 50° C. using [Rh(cod)(PP)]OTf (277 μmol) as catalyst and (S,S)-Et-Duphos as ligand (277 μmol). The solution was cooled and evaporated and the crude product was dissolved in EtOAc (550 mL). The mixture was heated to 60° C. followed by the slow addition of heptane (200 mL) before slowly cooling to ambient temperature. The solids were isolated to afford (Z)-2-acetylamino-3-(2-fluoro-4-methoxyphenyl)acrylic acid. RT=1.21 min (LCMS method 2). A tantal autoclave was charged with (Z)-2-acetylamino-3-(2-fluoro-4-methoxyphenyl)acrylic acid (71.0 g, 278 mmol), aqueous hydrobromic acid (48%, 420 mL) and acetic acid (320 mL) and heated to 105° C. for 16 h. The solvent was evaporated and the residue was subsequently triturated with diethyl ether and tert-butylmethylether before being dried at 30° C. in vacuo for 3 h to afford the title compound: RT=0.815 min; m/z (ES⁺)=200.0 [M+H]⁺(LCMS method 2).

### Preparation 28

### (S)-2-tert-Butoxycarbonylamino-3-(2-fluoro-4-hydroxyphenyl)propionic acid

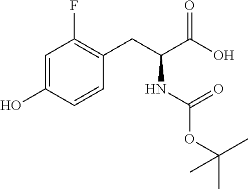

To a suspension of (S)-2-amino-3-(2-fluoro-4-hydroxyphenyl)propionic acid hydrobromide (Preparation 27, 15 g, 53.5 mmol) and triethylamine (15.64 mL, 112.25 mmol) in a mixture of dioxane (150 mL) and water (150 mL) at 0° C. was added di-tert-butyl dicarbonate (12.84 g, 58.85 mmol). The resulting suspension was allowed to warm to r.t. and stirred for 48 h before removing the dioxane in vacuo. The resulting residue was partitioned between EtOAc and water then the aqueous phase was separated and acidified to pH3 with 1M citric acid solution. The product was extracted into EtOAc, dried (MgSO₄) and the solvent removed in vacuo to afford the title compound: RT=2.82 min, m/z (ES⁺)=300.1 [M+H]⁺.

### Preparation 29

### (S)-[2-((S)-2-Carbamoylpyrrolidin-1-yl)-1-(2-fluoro-4-hydroxybenzyl)-2-oxoethyl]carbamic acid tert-butyl ester

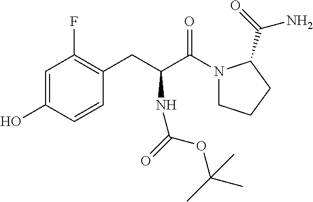

To a solution of (S)-2-tert-butoxycarbonylamino-3-(2-fluoro-4-hydroxyphenyl)propionic acid (Preparation 28, 15.3 g, 51 mmol) in THF (200 mL) was added EDCI (12.21 g, 63.75 mmol), HOBt (6.94 g, 51 mmol) and DIPEA (17.73 mL, 102 mmol) and the reaction was stirred for 20 min. The mixture was treated with L-(−)-prolinamide (6.51 g, 56.1 mmol) and stirring continued at r.t. for 18 h. The solvent was removed in vacuo and the resulting residue was diluted with DCM. The organic solution was washed with 2M Na₂CO₃ solution, followed by 0.1M citric acid solution before being dried (MgSO₄). Removal of the solvent in vacuo afforded the title compound: RT=2.67 min, m/z (ES⁺)=396.3 [M+H]⁺.

### Preparation 30

### (S)-Trifluoromethanesulfonic acid 4-[2-tert-butoxycarbonylamino-3-((S)-2-carbamoylpyrrolidin-1-yl)-3-oxopropyl]-3-fluorophenyl ester

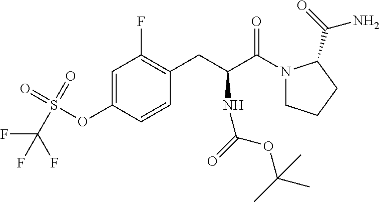

To a solution of (S)-[2-((S)-2-carbamoylpyrrolidin-1-yl)-1-(2-fluoro-4-hydroxybenzyl)-2-oxoethyl]carbamic acid tert-butyl ester (Preparation 29, 17.65 g, 44.46 mmol) and DIPEA (8.5 mL, 48.91 mmol) in MeCN at 0° C. was added N-phenyltrifluoromethane sulfonimide (15.94 g, 44.46 mmol). The reaction was stirred at r.t. for 2 h before removal of the solvent in vacuo. Purification of the residue by column chromatography (SiO₂, EtOAc) afforded the title compound: RT=3.42 min, m/z (ES⁺)=528.3 [M+H]⁺.

### Preparation 31

### (S)-Trifluoromethanesulfonic acid 4-[2-tert-butoxycarbonylamino-3-((S)-2-cyanopyrrolidin-1-yl)-3-oxopropyl]-3-fluorophenyl ester

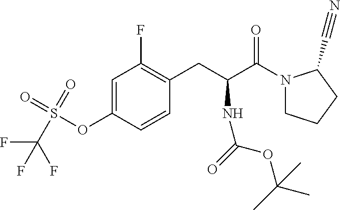

To a solution of (S)-trifluoromethanesulfonic acid 4-[2-tert-butoxycarbonylamino-3-((S)-2-carbamoylpyrrolidin-1-yl)-3-oxopropyl]-3-fluorophenyl ester (Preparation 30, 7.42 g, 14.08 mmol) in THF (150 mL) was added pyridine (2.26 mL, 28.16 mmol) and the mixture was cooled to 0° C. The reaction was treated with TFAA (9.78 mL, 70.4 mmol) and stirred for 5 min before being diluted with DCM (100 mL), washed with sat. Na₂CO₃ solution and dried (MgSO₄). Removal of the solvent in vacuo followed by purification by column chromatography (SiO₂, IH:EtOAc, 4:1) afforded the title compound: ¹H NMR δ_{H} (400 MHz, DMSO-d₆): 7.59-7.48 (m, 2H), 7.35-7.22 (m, 2H), 4.76-4.70 (m, 1H), 4.52-4.42 (m, 1H), 3.56-3.34 (m, 2H), 3.11-2.99 (m, 1H), 2.91-2.81 (m, 1H), 2.23-1.84 (m, 4H), 1.24 (s, 9H).

### Preparation 32

### (S)-[1-(2-Fluoro-4-hydroxybenzyl)-2-oxo-2-pyrrolidin-1-ylethyl]-carbamic acid tert-butyl ester

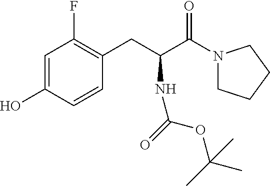

The title compound was prepared by reacting (S)-2-tert-butoxycarbonylamino-3-(2-fluoro-4-hydroxyphenyl)propionic acid (Preparation 28, 4.2 g, 14 mmol) with pyrrolidine (1.27 mL, 15.4 mmol) employing the procedure outlined in Preparation 29: RT=2.97 min, m/z (ES⁺)=353.3 [M+H]⁺.

### Preparation 33

### (S)-Trifluoromethanesulfonic acid-4-(2-tert-butoxycarbonylamino-3-oxo-3-pyrrolidin-1-ylpropyl)-3-fluorophenyl ester

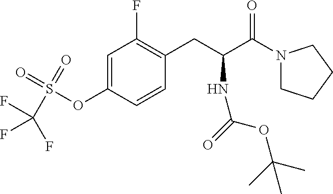

The title compound was prepared by reacting (S)-[1-(2-fluoro-4-hydroxybenzyl)-2-oxo-2-pyrrolidin-1-ylethyl]carbamic acid tert-butyl ester (Preparation 32, 5 g, 14.0 mmol) with N-phenyltrifluoromethane sulfonimide (5.5 g, 15.4 mmol) employing the procedure outlined in Preparation 30: RT=3.92 min, m/z (ES⁺)=485.3 [M+H]⁺.

### Preparation 34

### (S)-[1-(2-Fluoro-4-hydroxybenzyl)-2-((S)-3-fluoropyrrolidin-1-yl)-2-oxoethyl]carbamic acid tert-butyl ester

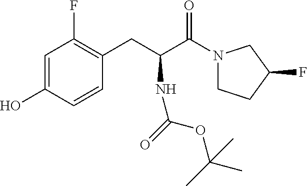

The title compound was prepared by reacting (S)-2-tert-butoxycarbonylamino-3-(2-fluoro-4-hydroxyphenyl)propionic acid (Preparation 28) with (S)-3-fluoropyrrolidine hydrochloride employing the procedure outlined in Preparation 29: RT=2.96 min, m/z (ES⁺)=371.2 [M+H]⁺.

### Preparation 35

### (S)-Trifluoromethanesulfonic acid 4-[2-tert-butoxycarbonylamino-3-((S)-3-fluoropyrrolidin-1-yl)-3-oxopropyl]3-fluorophenyl ester

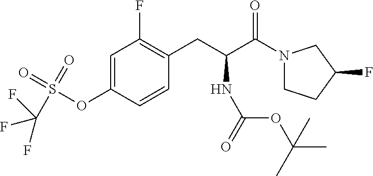

The title compound was prepared by reacting (S)-[1-(2-fluoro-4-hydroxybenzyl)-2-((S)-3-fluoropyrrolidin-1-yl)-2-oxoethyl]carbamic acid tert-butyl ester (Preparation 34, 4.42 g, 11.94 mmol) with N-phenyltrifluoromethane sulfonimide (4.6 g, 12.87 mmol) employing the procedure outlined in Preparation 30: RT=3.77 min, m/z (ES⁺)=503.1 [M+H]⁺.

### Preparation 36

### (S)-[2-(3,3-Difluoropyrrolidin-1-yl)-1-(2-fluoro-4-hydroxybenzyl)-2-oxoethyl]carbamic acid tert-butyl ester

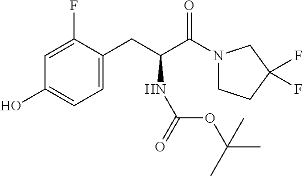

The title compound was prepared by reacting (S)-2-tert-butoxycarbonylamino-3-(2-fluoro-4-hydroxyphenyl)propionic acid (Preparation 28) with 3,3-difluoropyrrolidine hydrochloride employing a procedure similar to that outlined in Preparation 29 but using triethylamine as the base: RT=3.13 min, m/z (ES⁺)=389.2 [M+H]⁺.

### Preparation 37

### (S)-Trifluoromethanesulfonic acid 4-[2-tert-butoxycarbonylamino-3-(3,3-difluoropyrrolidin-1-yl)-3-oxopropyl]3-fluorophenyl ester

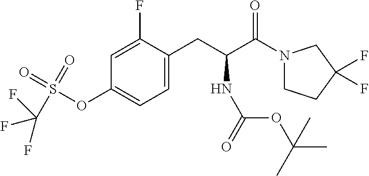

The title compound was prepared by reacting (S)-[2-(3,3-difluoropyrrolidin-1-yl)-1-(2-fluoro-4-hydroxybenzyl)-2-oxoethyl]carbamic acid tert-butyl ester (Preparation 36, 4.42 g, 11.94 mmol) with N-phenyltrifluoromethane sulfonimide (4.6 g, 12.87 mmol) employing the procedure outlined in Preparation 30: RT=3.93 min, m/z (ES⁺)=521.1 [M+H]⁺.

### Preparation 38

### (S)-4-(5-{4-[2-tert-Butoxycarbonylamino-3-(3,3-difluoropyrrolidin-1-yl)-3-oxopropyl]-3-fluorophenyl}pyridin-2-yloxy)piperidine-1-carboxylic acid isopropyl ester

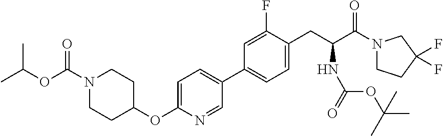

To a solution of 4-[5-(4,4,5,5-tetramethyl-[1,3,2]dioxaborolan-2-yl)pyridine-2-yloxy]piperidine-1-carboxylic acid isopropyl ester (Preparation 17, 250 mg, 0.64 mmol) in a mixture of DMF (4.0 mL) and water (1.3 mL) was added (S)-trifluoromethanesulfonic acid 4-[2-tert-butoxycarbonylamino-3-(3,3-difluoropyrrolidin-1-yl)-3-oxopropyl]3-fluorophenyl ester (Preparation 37, 278 mg, 0.53 mmol), [1,1-bis(diphenylphosphino)ferrocene]dichloropalladium (44 mg, 0.05 mmol) and triethylamine (223 μL, 1.6 mmol) and the reaction was heated in a microwave reactor at 80° C. for 20 min. The mixture was taken up into EtOAc, washed with brine and dried (MgSO₄). Removal of the solvent in vacuo and purification by column chromatography (SiO₂, 1H:EtOAc, 100:0, 90:10, 80:20, 70:30, 50:50, 0:100) afforded the title compound: RT=4.23 min; m/z (ES⁺)=635.3 [M+H]⁺.

### Preparation 39

### (S)-4-(5-{4-[2-tert-Butoxycarbonylamino-3-((S)-3-fluoropyrrolidin-1-yl)-3-oxopropyl]-3-fluorophenyl}pyridin-2-yloxy)piperidine-1-carboxylic acid isopropyl ester

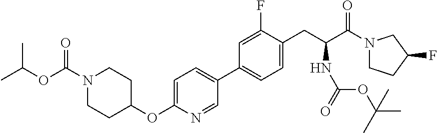

The title compound was prepared from 4-[5-(4,4,5,5-tetramethyl-[1,3,2]dioxaborolan-2-yl)pyridine-2-yloxy]piperidine-1-carboxylic acid isopropyl ester (Preparation 17, 200 mg, 0.51 mmol) and (S)-trifluoromethanesulfonic acid 4-[2-tert-butoxycarbonylamino-3-((S)-3-fluoropyrrolidin-1-yl)-3-oxopropyl]-3-fluorophenyl ester (Preparation 35, 214 mg, 0.43 mmol) employing the procedure outlined in Preparation 38: RT=4.10 min; m/z (ES⁺)=617.4 [M+H]⁺.

### Preparation 40

### (S)-4-(5-{4-[2-tert-Butoxycarbonylamino-3-((S)-2-cyanopyrrolidin-1-yl)-3-oxopropyl]-3-fluorophenyl}pyridin-2-yloxymethyl)piperidine-1-carboxylic acid isopropyl ester

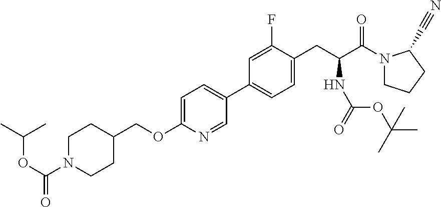

The title compound was prepared from 4-[5-(4,4,5,5-tetramethyl-[1,3,2]dioxaborolan-2-yl)pyridin-2-yloxymethyl]piperidine-1-carboxylic acid isopropyl ester (Preparation 18, 240 mg, 0.59 mmol) and (S)-trifluoromethanesulfonic acid 4-[2-tert-butoxycarbonylamino-3-((S)-2-cyanopyrrolidin-1-yl)-3-oxopropyl]3-fluorophenyl ester (Preparation 31, 250 mg, 0.49 mmol) employing the procedure outlined in Preparation 38: RT=4.32 min; m/z (ES⁺)=638.5 [M+H]⁺.

### Preparation 41

### (S)-4-(5-{4-[2-tert-Butoxycarbonylamino-3-((S)-3-fluoropyrrolidin-1-yl)-3-oxopropyl]-3-fluorophenyl}pyridin-2-yloxymethyl)piperidine-1-carboxylic acid isopropyl ester

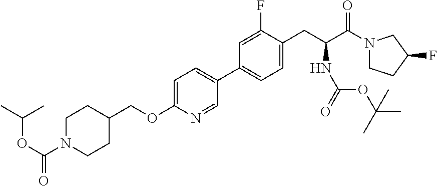

The title compound was prepared from 4-[5-(4,4,5,5-tetramethyl-[1,3,2]dioxaborolan-2-yl)pyridin-2-yloxymethyl]piperidine-1-carboxylic acid isopropyl ester (Preparation 18, 242 mg, 0.60 mmol) and (S)-trifluoromethanesulfonic acid 4-[2-tert-butoxycarbonylamino-3-((S)-3-fluoropyrrolidin-1-yl)-3-oxopropyl]3-fluorophenyl ester (Preparation 35, 250 mg, 0.50 mmol) employing the procedure outlined in Preparation 38: RT=4.25 min; m/z (ES⁺)=631.5 [M+H]⁺.

### Preparation 42

### [2-((S)-3-Fluoropyrrolidin-1-yl)-1-(4-hydroxyphenyl)-2-oxoethyl]carbamic acid tert-butyl ester

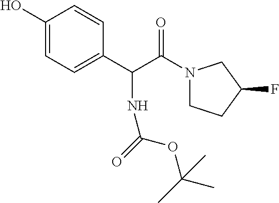

To a solution of tert-butoxycarbonylamino(4-hydroxyphenyl)acetic acid (3.0 g, 11.22 mmol) in a mixture of DCM (30 mL) and DMF (6 mL) under argon was added HOBt (2.06 g, 13.47 mmol) and EDCI (2.80 g, 14.59 mmol) and the mixture was stirred at r.t. for 15 min. To the reaction was added (S)-3-fluoropyrrolidine hydrochloride (1.55 g, 12.35 mmol) and DIPEA (4.89 mL, 28.06 mmol) and stirring continued for a further 16 h. Solvent was removed in vacuo and the crude material was partitioned between EtOAc (150 mL) and water (100 mL). The aqueous layer was removed and the organic phase was washed with water (100 mL), NaHCO₃ solution (100 mL), 1M HCl solution (100 mL) then brine (100 mL) before being dried (MgSO₄). Removal of the solvent in vacuo followed by column chromatography (SiO₂, DCM:MeOH, 95:5) afforded the title compound: RT=2.76 min; m/z (ES⁺)=339.3 [M+H]⁺.

### Preparation 43

### Trifluoromethanesulfonic acid 4-[1-tert-butoxycarbonylamino-2-((S)-3-fluoropyrrolidin-1-yl)-2-oxoethyl]phenyl ester

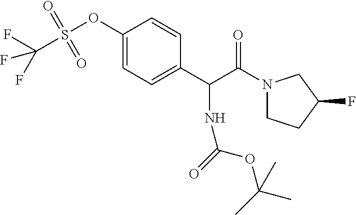

A solution of [2-((S)-3-fluoropyrrolidin-1-yl)-1-(4-hydroxyphenyl)-2-oxoethyl]carbamic acid tert-butyl ester (Preparation 42, 2.28 g, 6.74 mmol) in MeCN (100 mL) under argon, was cooled to 0° C. DIPEA (1.29 mL, 7.41 mmol) was added, followed by N-phenyltrifluoromethane sulfonimide (2.65 g, 7.41 mmol) and the reaction was stirred at 0° C. for 10 min before being allowed to stir at r.t. for 16 h. The reaction solvent was removed in vacuo and the crude residue was re-dissolved in EtOAc (150 mL). The organic solution was washed with sat. NaHCO₃ solution (60 mL), then brine (60 mL) and dried (MgSO₄) and the solvent was removed in vacuo. Purification by column chromatography (SiO₂, 1H:EtOAc, 1:1) afforded the title compound: RT=3.74 min; m/z (ES⁺)=471.3 [M+H]⁺.

### Preparation 44

### (S)-4-{4′-[1-tert-Butoxycarbonylamino-2-((S)-3-fluoropyrrolidin-1-yl)-2-oxoethyl]biphenyl-4-yloxy}piperidine-1-carboxylic acid isopropyl ester

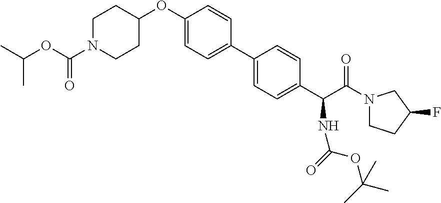

To a solution of trifluoromethanesulfonic acid 4-[1-tert-butoxycarbonylamino-2-((S)-3-fluoropyrrolidin-1-yl)-2-oxoethyl]phenyl ester (Preparation 43, 544 mg, 1.16 mmol) and 4-[4-(4,4,5,5-tetramethyl-[1,3,2]dioxaborolan-2-yl)-phenoxy]piperidine-1-carboxylic acid isopropyl ester (Preparation 19, 300 mg, 0.77 mmol) in a mixture of toluene (5 mL) and EtOH (5 mL), under argon, was added Na₂CO₃ solution (2M, 1.93 mL, 3.85 mmol) and the mixture was stirred for 5 min. [1,1-Bis(diphenylphosphino)ferrocene]dichloropalladium (126 mg, 0.15 mmol) was added and the reaction was bubbled with argon for 5 min before heating to 80° C. for 16 h. The reaction solvent was concentrated in vacuo and the resulting residue was partitioned between EtOAc (100 mL) and water (100 mL). The organic layer was separated and washed with water (50 mL), saturated NaHCO₃ solution (100 mL) then brine (100 mL) and dried (MgSO₄). Removal of the solvent in vacuo followed by purification by column chromatography (SiO₂, 1H:EtOAc, 1:1) and chiral HPLC afforded the title compound: RT=4.06 min; m/z (ES⁺)=584.6 [M+H]⁺.

### Preparation 45

### [2-((S)-2-Carbamoylpyrrolidin-1-yl)-1-(4-hydroxyphenyl)-2-oxoethyl]carbamic acid tert-butyl ester

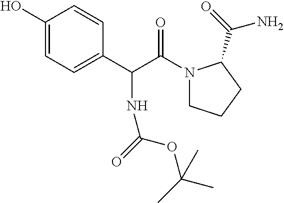

To a solution of tert-butoxycarbonylamino(4-hydroxyphenyl)acetic acid (3.0 g, 11.2 mmol) in DCM (75 mL) was added (S)-prolinamide (1.54 g, 13.5 mmol), EDCI (2.15 g, 11.2 mmol), HOBt (2.06 g, 13.47 mmol) and DIPEA (4.69 mL, 26.9 mmol) and the reaction was stirred at r.t. for 24 h. The reaction mixture was diluted with DCM and washed with 1M citric acid, sat. NaHCO₃ solution, water then brine and dried (MgSO₄). Removal of the solvent in vacuo and purification by column chromatography (SiO₂, DCM:MeOH, 95:5, 90:10) afforded the title compound: RT=2.50 min; m/z (ES⁺)=364.3 [M+H]⁺.

### Preparation 46

### Trifluoromethanesulfonic acid 4-[1-tert-butoxycarbonylamino-2-((S)-2-carbamoylpyrrolidin-1-yl)-2-oxoethyl]phenyl ester

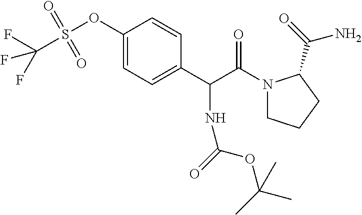

The title compound was prepared from [2-((S)-2-carbamoylpyrrolidin-1-yl)-1-(4-hydroxyphenyl)-2-oxoethyl]carbamic acid tert-butyl ester (Preparation 45, 1.93 g, 3.13 mmol) employing the procedure outlined in Preparation 43: RT=3.37 min; m/z (ES⁺)=496.4 [M+H]⁺.

### Preparation 47

### Trifluoromethanesulfonic acid 4-[1-tert-butoxycarbonylamino-2-((S)-cyanopyrrolidin-1-yl)-2-oxoethyl]phenyl ester

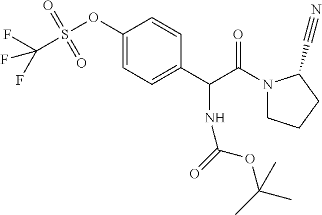

To a solution of trifluoromethanesulfonic acid 4-[1-tert-butoxycarbonylamino-2-((S)-2-carbamoylpyrrolidin-1-yl)-2-oxoethyl]phenyl ester (Preparation 46, 1.55 g, 3.13 mmol) in THF (60 mL), cooled to 0° C., was added pyridine (0.53 mL, 6.57 mmol) followed by TFAA (2.39 mL, 16.89 mmol). The reaction mixture was stirred at 0° C. for 5 min and then quenched by the addition of sat. NaHCO₃ solution. Organics were extracted into EtOAc and washed with 1M citric acid, water, then brine, and dried (MgSO₄). Removal of the solvent in vacuo followed by purification by column chromatography (SiO₂, 1H:EtOAc, 1:1 followed by a second column using DCM:EtOAc, 95:5) afforded the title compound: RT=3.80 min; m/z (ES⁺)=478.4 [M+H]⁺.

### Preparation 48

### (S)-4-{4′-[1-tert-Butoxycarbonylamino-2-((S)-2-cyanopyrrolidin-1-yl)-2-oxoethyl]biphenyl-4-yloxy}piperidine-1-carboxylic acid isopropyl ester

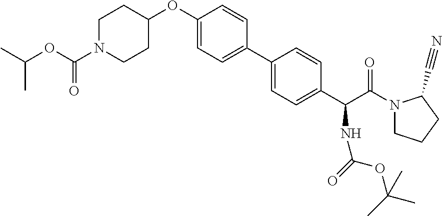

4-[4-(4,4,5,5-Tetramethyl-[1,3,2]dioxaborolan-2-yl)-phenoxy]piperidine-1-carboxylic acid isopropyl ester (Preparation 19, 234 mg, 0.6 mmol) was reacted with trifluoromethanesulfonic acid 4-[1-tert-butoxycarbonylamino-2-((S)-cyanopyrrolidin-1-yl)-2-oxoethyl]phenyl ester (Preparation 47, 240 mg, 0.5 mmol) employing a similar procedure to that outlined in Preparation 38. Purification by chiral HPLC afforded the title compound: RT=4.17 min; m/z (ES⁺)=591.3 [M+H]⁺.

### Preparation 49

### (S)-4-[4′-(2-tert-Butoxycarbonylamino-3-oxo-3-pyrrolidin-1-yl-propyl)-3′-fluorobiphenyl-4-yloxymethyl]piperidine-1-carboxylic acid isopropyl ester

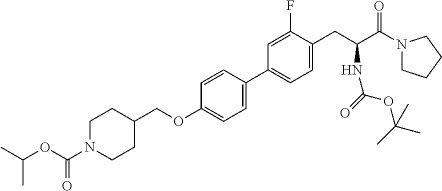

A solution of 4-[4-(4,4,5,5-tetramethyl[1,3,2]dioxaborolan-2-yl)phenoxymethyl]piperidine-1-carboxylic acid isopropyl ester (Preparation 20, 200 mg, 0.50 mmol) in a mixture of DMF (1.5 mL) and water (0.5 mL) was combined with (S)-trifluoromethanesulfonic acid-4-(2-tert-butoxycarbonylamino-3-oxo-3-pyrrolidin-1-ylpropyl)-3-fluorophenyl ester (Preparation 33, 200 mg, 0.41 mmol), [1,1-bis(diphenylphosphino)ferrocene]dichloropalladium (34 mg, 0.04 mmol) and DIPEA (216 μL, 1.24 mmol). The reaction was heated in a microwave reactor at 80° C. for 20 min. EtOAc (100 mL) was added and the solution was washed with water (3×50 mL), sat. NaHCO₃ solution (2×50 mL) then brine (50 mL), and dried (MgSO₄). Removal of the solvent in vacuo followed by purification by column chromatography (SiO₂, 1H:EtOAc, 1:1) afforded the title compound: RT=4.53 min; m/z (ES⁺)=612.5 [M+H]⁺.

The following compounds were prepared by reacting 4-[4-(4,4,5,5-tetramethyl-[1,3,2]dioxaborolan-2-yl)phenoxymethyl]piperidine-1-carboxylic acid isopropyl ester (Preparation 20) with the appropriate trifluoromethanesulfonate ester intermediate employing the procedure outlined in Preparation 49:

| Prep | | | |
|---|---|---|---|
| No. | Structure | Name | LCMS |
| 50 | 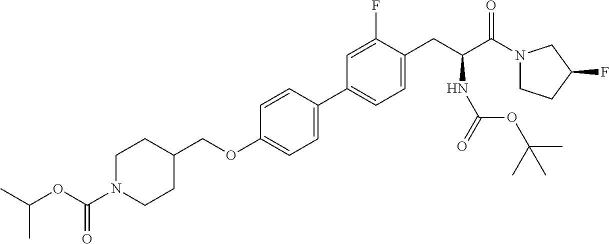 | (S)-4-{4′-{2-tert- Butoxycarbonylamino-3- ((S)-3-fluoropyrrolidin- 1-yl)-3-oxopropyl]-3′- fluorobiphenyl-4- yloxymethyl}piperidine- 1-carboxylic acid isopropyl ester | RT = 4.35 min; m/z (ES⁺) = 360.5 [M + H]⁺ |
| 51 | 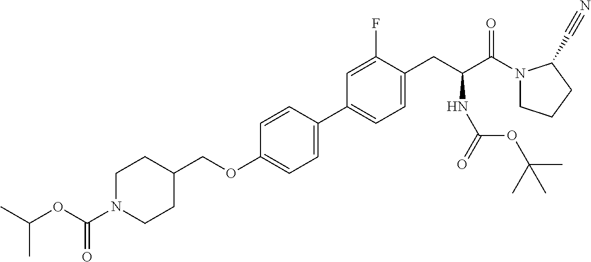 | (S)-4-{4′-{2-tert- Butoxycarbonylamino-3- ((S)-2-cyanopyrrolidin- 1-yl)-3-oxopropyl]-3′- fluorobiphenyl-4- yloxymethyl}piperidine- 1-carboxylic acid isopropyl ester | RT = 4.43 min; m/z (ES⁺) = 637.5 [M + H]⁺ |

### Preparation 52

### 4-(6-Bromopyridin-3-yloxymethyl)piperidine-1-carboxylic acid isopropyl ester

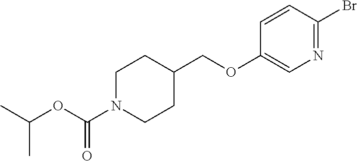

To a dry solution of azodicarboxylic dipiperidide (5.01 g, 19.87 mmol) in toluene (200 mL) was added tri n-butylphosphine (4.95 mL, 19.87 mmol) followed by 2-bromo-5-hydroxypyridine (1.73 g, 9.94 mmol) and the reaction was cooled to 0° C. for 5 min. A solution of 4-hydroxymethyl piperidine-1-carboxylic acid isopropyl ester (Preparation 1, 2.0 g, 9.94 mmol) in toluene (50 mL) was added to the solution, dropwise over 5 min, and the reaction was allowed to stir at 0° C. for 16 h, iso-hexane (200 mL) was added and the reaction was stirred for 10 min before filtering the mixture to remove the precipitate. The filtrate was washed with 2M NaOH solution (2×150 mL), water (150 mL), 1M HCl solution (2×150 mL), brine (150 mL) and dried (MgSO₄). Removal of the solvent in vacuo and purification by column chromatography (SiO₂, IH:EtOAc, 4:1, 3:1) afforded the title compound: RT=3.87 min; m/z (ES⁺)=357.2, 359.1 [M+H]⁺.

### Preparation 53

### (S)-{1-[2-Fluoro-4-(4,4,5,5-tetramethyl-[1,3,2]dioxaborolan-2-yl)benzyl]-2-oxo-2-pyrrolidin-1-ylethyl}carbamic acid tert-butyl ester

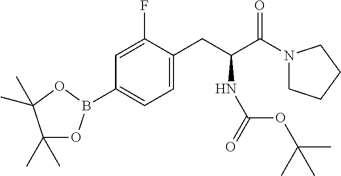

To a solution of (S)-trifluoromethanesulfonic acid-4-(2-tert-butoxycarbonylamino-3-oxo-3-pyrrolidin-1-ylpropyl)-3-fluorophenyl ester (Preparation 33, 320 mg, 0.66 mmol) in dioxane (12 mL) under argon was added bis(pinacolato)diboron (201 mg, 0.79 mmol), potassium acetate (194 mg, 0.07 mmol) and 1,1-bis(diphenylphosphino)ferrocene (37 mg, 0.07 mmol) and the mixture was bubbled with argon for 5 min. [1,1-bis(diphenylphosphino)ferrocene]dichloropalladium (54 mg, 0.07 mmol) was added and the reaction was bubbled with argon for a further 10 min before being heated to 90° C. for 16 h. The reaction mixture was filtered through celite then purification by column chromatography (SiO₂, 1H:EtOAc, 6:4) afforded the title compound: RT=4.07 min; m/z (ES⁺)=463.4 [M+H]⁺.

The following compounds were prepared by reacting bis(pinacolato)diboron with the appropriate trifluoromethanesulfonate ester intermediate employing the procedure outlined in

### Preparation 53

| Prep | | | |
|---|---|---|---|
| No. | Structure | Name | LCMS |
| 54 | 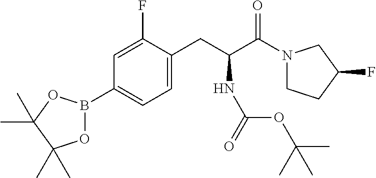 | (S)-{2-((S)-3- Fluoropyrrolidin-1- yl)-1-[2-fluoro-4- (4,4,5,5-tetramethyl- [1,3,2]dioxaborolan- 2-yl)benzyl]-2- oxoethyl}carbamic acid tert-butyl ester | RT = 3.86 min; m/z (ES⁺) = 481.4 [M + H]⁺ |
| 55 | 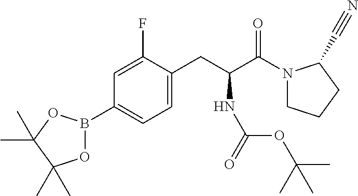 | (S)-{2-((S)-2- Cyanopyrrolidin-1- yl)-1-[2-fluoro-4- (4,4,5,5-tetramethyl- [1,3,2]dioxaborolan- 2-yl)benzyl]-2- oxoethyl}carbamic acid tert-butyl ester | RT = 4.03 min; m/z (ES⁺) = 488.4 [M + H]⁺ |

### Preparation 56

### (S)-4-{6-[4-(2-tert-Butoxycarbonylamino-3-oxo-3-pyrrolidin-1-ylpropyl)-3-fluorophenyl]pyridin-3-yloxymethyl}piperidine-1-carboxylic acid isopropyl ester

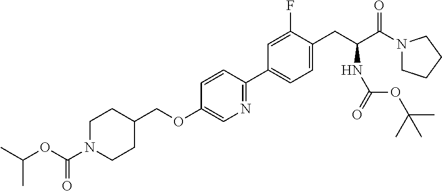

4-(6-Bromopyridin-3-yloxymethyl)piperidine-1-carboxylic acid isopropyl ester (Preparation 52, 133 mg, 0.37 mmol), (S)-{1-[2-fluoro-4-(4,4,5,5-tetramethyl-[1,3,2]dioxaborolan-2-yl)benzyl]-2-oxo-2-pyrrolidin-1-ylethyl}carbamic acid tert-butyl ester (Preparation 53, 190 mg, 0.41 mmol), [1,1-bis(diphenylphosphino)ferrocene]dichloropalladium (31 mg, 0.04 mmol) and DIPEA (195 mg, 1.12 mmol) were combined in a mixture of DMF (1.5 mL) and water (0.5 mL) and the reaction was heated to 80° C. in a microwave reactor for 25 min. The reaction mixture was diluted with EtOAc (50 mL) then washed with water (3×30 mL), sat. NaHCO₃ solution (2×30 mL) then brine (30 mL) and dried (MgSO₄). Removal of the solvent in vacuo followed by purification by column chromatography (SiO₂, DCM:EtOAc, 7:3) afforded the title compound: RT=4.10 min; m/z (ES⁺)=613.5 [M+H]⁺.

The following compounds were prepared by reacting 4-(6-bromopyridin-3-yloxymethyl)-piperidine-1-carboxylic acid isopropyl ester (Preparation 52) with the appropriate boronate ester intermediate employing the procedure outlined in Preparation 56:

| Prep | | | |
|---|---|---|---|
| No. | Structure | Name | LCMS |
| 57 | 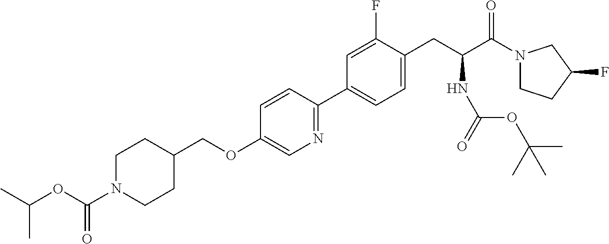 | (S)-4-(6-{4-[2-tert- Butoxycarbonylamino-3- ((S)-3-fluoropyrrolidin- 1-yl)-3-oxopropyl]-3- fluorophenyl}pyridin-3- yloxymethyl)piperidine- 1-carboxylic acid isopropyl ester | RT = 4.02 min; m/z (ES⁺) = 631.5 [M + H]⁺ |
| 58 | 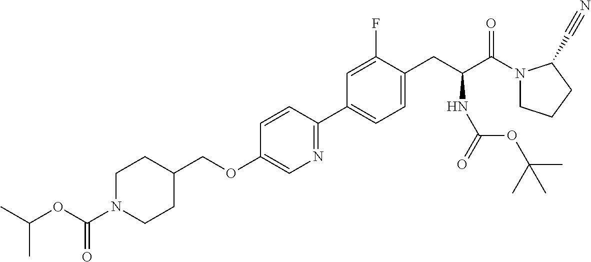 | (S)-4-(6-{4-(2-tert- Butoxycarbonylamino-3- ((S)-2-cyanopyrrolidin- 1-yl)-3-oxopropyl]-3- fluorophenyl}pyridin-3- yloxymethyl)piperidine- 1-carboxylic acid isopropyl ester | RT = 4.15 min; m/z (ES⁺) = 638.5 [M + H]⁺ |

### Preparation 59

### (S)-[1-(2-Fluoro-4-{6-[1-(4-isopropylbenzypazetidin-3-yloxy]pyridin-3-yl}benzyl)-2-oxo-2-pyrrolidin-1-yl ethyl]carbamic acid tert-butyl ester

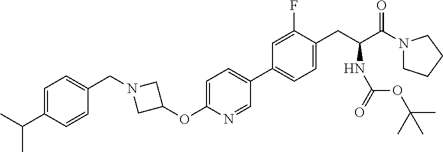

The title compound was prepared from 5-bromo-2-[1-(4-isopropylbenzyl)azetidin-3-yloxy]pyridine (Preparation 16, 92 mg, 0.25 mmol) and (S)-{1-[2-fluoro-4-(4,4,5,5-tetramethyl-[1,3,2]dioxaborolan-2-yl)benzyl]-2-oxo-2-pyrrolidin-1-yl ethyl}carbamic acid tert-butyl ester (preparation 53, 140 mg, 0.3 mmol) employing the procedure outlined in Preparation 38: RT=3.23 min; m/z (ES⁺)=617.5 [M+H]⁺.

### Preparation 60

### (S)-[2-((S)-Cyanopyrrolidin-1-yl)-1-(2-fluoro-4-{6-[1-(4-isopropylbenzyl)azetidin-3-yloxy]pyridin-3-yl}benzyl)-2-oxoethyl]carbamic acid tert-butyl ester

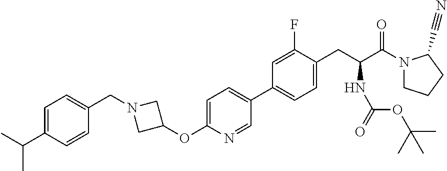

The title compound was prepared from 5-bromo-2-[1-(4-isopropylbenzyl)azetidin-3-yloxy]pyridine (Preparation 16, 111.1 mg, 0.31 mmol) and (S)-{2-((S)-2-cyanopyrrolidin-1-yl)-1-[2-fluoro-4-(4,4,5,5-tetramethyl-[1,3,2]dioxaborolan-2-yl)benzyl]-2-oxoethyl}carbamic acid tert-butyl ester (Preparation 55, 180 mg, 0.37 mmol) employing the procedure outlined in Preparation 38: RT=3.22 min; m/z (ES⁺)=642.5 [M+H]⁺.

### Preparation 61

### (S)-4-[1-(5-{4-[2-tert-Butoxycarbonylamino-3-((S)-2-cyanopyrrolidin-1-yl)-3-oxopropyl]-3-fluorophenyl}pyridin-2-yloxy)ethyl]piperidine-1-carboxylic acid isopropyl ester

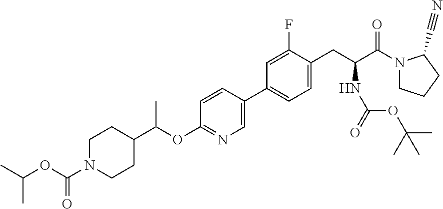

4-{1-[5-(4,4,5,5-Tetramethyl-[1,3,2]dioxaborolan-2-yl)pyridin-2-yloxy]ethyl}piperidine-1-carboxylic acid isopropyl ester (Preparation 21, 49 mg, 0.12 mmol), (S)-trifluoromethanesulfonic acid 4-[2-tert-butoxycarbonylamino-3-((S)-2-cyanopyrrolidin-1-yl)-3-oxopropyl]-3-fluorophenyl ester (Preparation 31, 50 mg, 0.1 mmol), [1,1-bis(diphenylphosphino)ferrocene]dichloropalladium (8 mg, 0.01 mmol) and triethylamine (41 μL, 0.29 mmol) were combined in a mixture of DMF (0.75 mL) and water (0.25 mL), and the reaction was heated in a microwave reactor at 80° C. for 3×20 min. The mixture was then filtered through celite, washing with EtOAc. The filtrate was washed with water (×4), 1M citric acid, then brine and dried (MgSO₄). Removal of the solvent in vacuo followed by purification by column chromatography (SiO₂, 1H:EtOAc, 1:1) afforded the title compound: RT=4.43 min; m/z (ES⁺)=652.6 [M+H]⁺.

The following compounds were prepared by reacting the appropriate aryl or heteroaryl boronate ester with the appropriate trifluoromethanesulfonate ester intermediate employing a similar procedure to that outlined in Preparation 61:

| Prep | | | |
|---|---|---|---|
| No. | Structure | Name | LCMS |
| 62 | 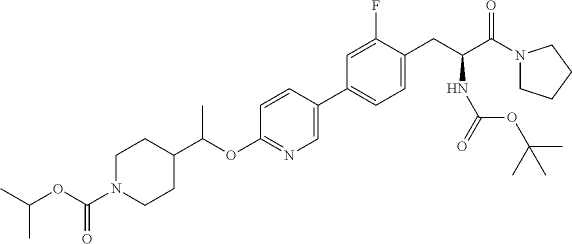 | (S)-4-(1-{5-[4-(2-tert- Butoxycarbonylamino-3- oxo-3-pyrrolidin-1- ylpropyl)-3- fluorophenyl]pyridin-2- yloxy}ethyl)piperidine- 1-carboxylic acid isopropyl ester | RT = 4.39 min; m/z (ES⁺) = 627.6 [M + H]⁺ |
| 63 | 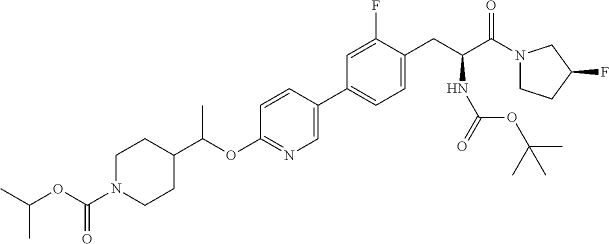 | (S)-4-[1-(5-{4-[2-tert- Butoxycarbonylamino-3- ((S)-3-fluoropyrrolidin- 1-yl)-3-oxopropyl]-3- fluorophenyl}pyridin-2- yloxy)ethyl]piperidine- 1-carboxylic acid isopropyl ester | RT = 4.35 min; m/z (ES⁺) = 645.6 [M + H]⁺ |
| 64 | 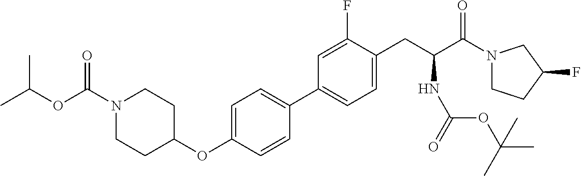 | (S)-4-{4′-[2-tert- Butoxycarbonylamino-3- ((S)-3-fluoropyrrolidin- 1-yl)-3-oxopropyl]-3′- fluorobiphenyl-4- yloxy}piperidine-1- carboxylic acid isopropyl ester | RT = 4.22 min; m/z (ES⁺) = 616.4 [M + H]⁺ |
| 65 | 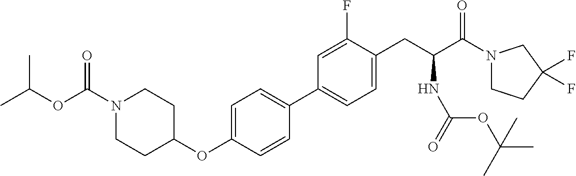 | (S)-4-{4′-[2-tert- Butoxycarbonylamino-3- (3,3-difluoropyrrolidin- 1-yl)-3-oxopropyl]- 3′fluorobiphenyl-4- yloxy}piperidine-1- carboxylic acid isopropyl ester | RT = 4.32 min; m/z (ES⁺) = 634.3 [M + H]⁺ |
| 66 | 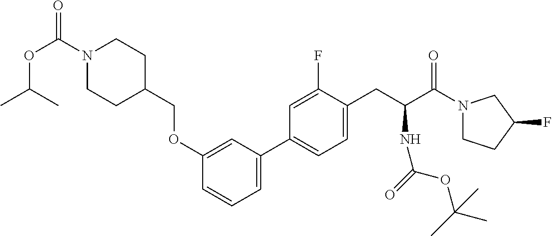 | (S)-4-{4′-[2-tert- Butoxycarbonylamino-3- ((S)-3-fluoropyrrolidin- 1-yl)-3-oxopropyl]-3′- fluorobiphenyl-3- yloxymethyl}piperidine- 1-carboxylic acid isopropyl ester | RT = 4.33 min; m/z (ES⁺) = 630.5 [M + H]⁺ |
| 67 | 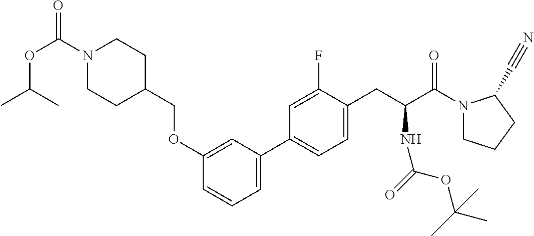 | (S)-4-{4′-[2-tert- Butoxycarbonylamino-3- ((S)-2-cyanopyrrolidin- 1-yl)-3-oxopropyl]-3′- fluorobiphenyl-3- yloxymethyl}piperidine- 1-carboxylic acid isopropyl ester | RT = 4.43 min; m/z (ES⁺) = 637.5 [M + H]⁺ |

### Preparation 68

### (S)-2-tert-Butoxycarbonylamino-3-(4-trifluoromethanesulfonyloxy-phenyl)propionic acid methyl ester

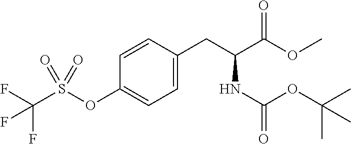

To a solution of (S)-2-tert-butoxycarbonylamino-3-(4-hydroxyphenyl)propionic acid methyl ester (2.90 g, 9.3 mmol) in MeCN (100 mL) was added N-phenyltrifluoromethane sulfonimide (4.98 g, 13.9 mmol) and DIPEA (1.94 mL, 11.1 mmol) and the mixture was stirred at r.t. for 12 h. After concentration in vacuo the residue was re-dissolved in EtOAc (400 mL), which was then washed with conc. NH₄Cl solution, water and brine before being dried (MgSO₄). Removal of the solvent in vacuo followed by purification by column chromatography (SiO₂, IH:EtOAc, 2:1) afforded the title compound: RT=4.01 min; m/z (ES⁺)=450.18 [M+Na]⁺.

### Preparation 69

### (S)-4-[4′-(2-tert-Butoxycarbonylamino-2-methoxycarbonylethyl)biphenyl-4-yloxymethyl]piperidine-1-carboxylic acid isopropyl ester

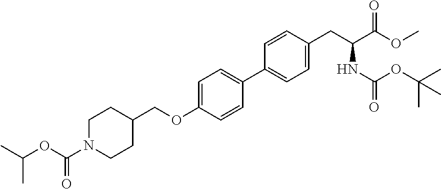

A mixture of (S)-2-tert-butoxycarbonylamino-3-(4-trifluoromethanesulfonyloxy-phenyl)propionic acid methyl ester (Preparation 68, 311 mg, 0.73 mmol), 4-[4-(4,4,5,5-tetramethyl[1,3,2]dioxaborolan-2-yl)phenoxymethyl]piperidine-1-carboxylic acid isopropyl ester (Preparation 20, 352 mg, 0.87 mmol), triethylamine (0.3 mL, 2.15 mmol) and [1,1-bis(diphenylphosphino)ferrocene]dichloropalladium (78 mg, 0.10 mmol) in a solution of DMF (4 mL) and water (1 mL) was heated in a microwave reactor at 80° C. for 20 min. The reaction mixture was diluted with EtOAc (200 mL) then washed with aqueous 1M HCl solution, water and brine before being dried (MgSO₄). Removal of the solvent in vacuo and purification of the residue by column chromatography (SiO₂, 1H:EtOAc, 2:1) afforded the title compound: RT=4.64 min; m/z (ES⁺)=572.46 [M+NH₄]⁺.

### Preparation 70

### (S)-4-[4′-(2-tert-Butoxycarbonylamino-2-carboxyethyl)biphenyl-4-yloxymethyl]piperidine-1-carboxylic acid isopropyl ester

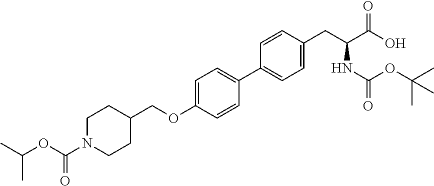

To a solution of (S)-4-[4′-(2-tert-butoxycarbonylamino-2-methoxycarbonylethyl)biphenyl-4-yloxymethyl]piperidine-1-carboxylic acid isopropyl ester (Preparation 69, 202 mg, 0.36 mmol) in THF (15 mL) at 0° C. was added water (3 mL) and lithium hydroxide hydrate (49 mg, 1.17 mmol). The ice bath was removed and stirring continued for 18 h at r.t. Water (50 mL) was added and the pH of the mixture was adjusted to 2-3 with dilute aqueous HCl. The reaction mixture was extracted with EtOAc, and the organic phase was washed with brine and dried (MgSO₄), then removal of the solvent in vacuo afforded the title compound: RT=4.15 min; m/z (ES⁺)=558.5 [M+NH₄]⁺.

### Preparation 71

### (S)-4-{4′-[2-tert-Butoxycarbonylamino-3-((S)-3-fluoropyrrolidin-1-yl)-3-oxopropyl]biphenyl-4-yloxymethyl}piperidine-1-carboxylic acid isopropyl ester

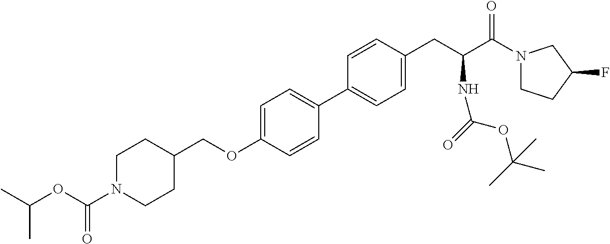

To a solution of (S)-4-[4′-(2-tert-butoxycarbonylamino-2-carboxyethyl)biphenyl-4-yloxymethyl]piperidine-1-carboxylic acid isopropyl ester (Preparation 70, 88 mg, 0.16 mmol) and (S)-(+)-3-fluoropyrrolidine hydrochloride (42 mg, 0.33 mmol) in DMF (5 mL) was added HOBt (26 mg, 0.17 mmol), EDCI (42 mg, 0.22 mmol) and DIPEA (0.12 mL, 0.69 mmol). After stirring at r.t. for 12 h the reaction mixture was partitioned between EtOAc (100 mL) and water/brine (1:1, 100 mL). The layers were separated and the aqueous phase was extracted with EtOAc (3×50 mL). The combined organic layers were washed with 1M HCl solution (50 mL), 1M NaOH solution (50 mL) and brine (50 mL) then dried (MgSO₄). Removal of the solvent in vacuo followed by purification by column chromatography (SiO₂, 1H:EtOAc, 2:3) afforded the title compound: RT=4.24 min; m/z (ES⁺)=612.5 [M+H]⁺.

### Preparation 72

### (S)-4-{4′-[2-tert-Butoxycarbonylamino-3-((S)-2-carbamoylpyrrolidin-1-yl)-3-oxo-propyl]biphenyl-4-yloxymethyl}piperidine-1-carboxylic acid isopropyl ester

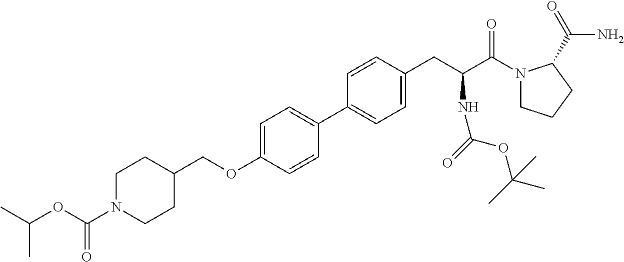

The title compound was prepared from (S)-4-[4′-(2-tert-butoxycarbonylamino-2-carboxyethyl)biphenyl-4-yloxymethyl]piperidine-1-carboxylic acid isopropyl ester (Preparation 70, 88 mg, 0.16 mmol) and L-(−)-prolinamide (40 mg, 0.35 mmol) employing the procedure outlined in Preparation 71: RT=3.98 min; m/z (ES⁺)=637.5 [M+H]⁺.

### Preparation 73

### (S)-4-{4′-[2-tert-Butoxycarbonylamino-3-((S)-2-cyanopyrrolidin-1-yl)-3-oxopropyl]biphenyl-4-yloxymethyl}piperidine-1-carboxylic acid isopropyl ester

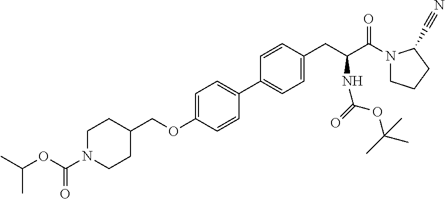

To a solution of (S)-4-{4′-[2-tert-butoxycarbonylamino-3-((S)-2-carbamoylpyrrolidin-1-yl)-3-oxopropyl]biphenyl-4-yloxymethyl}piperidine-1-carboxylic acid isopropyl ester (Preparation 72, 71 mg, 0.11 mmol) in dry THF (5 mL) at 0° C. was added pyridine (20 μL, 0.25 mmol) and TFAA (80 mL, 0.58 mmol). The mixture was stirred for 10 min at 0° C. before being quenched by adding to sat. NaHCO₃ solution (100 mL). Organics were extracted with EtOAc (3×50 mL). The combined extracts were dried (MgSO₄), filtered and concentrated in vacuo, then purification of the residue by column chromatography (SiO₂, 1H:EtOAc, 1:2) afforded the title compound: RT=4.35 min; m/z (ES⁺)=636.5 [M+NH₄]⁺

### Preparation 74

### (S)-{1-[2-Fluoro-4-(6-fluoropyridin-3-yl)benzyl]-2-oxo-2-pyrrolidin-1-ylethyl}carbamic acid tert-butyl ester

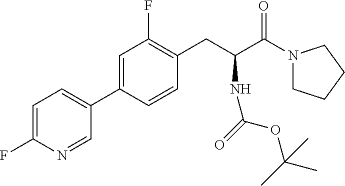

To a solution of (S)-trifluoromethanesulfonic acid-4-(2-tert-butoxycarbonylamino-3-oxo-3-pyrrolidin-1-ylpropyl)-3-fluorophenyl ester (Preparation 33, 650 mg, 1.34 mmol) in a mixture of THF and water (5:1, 17 mL) was added 2-fluoropyridyl-5-boronic acid (246 mg, 1.75 mmol), potassium phosphate (462 mg, 2.01 mmol) and [1,1-bis(diphenylphosphino)ferrocene]dichloropalladium (109 mg, 0.13 mmol), and the reaction was heated in a microwave reactor at 80° C. for 20 min. Additional portions of 2-fluoropyridyl-5-boronic acid (30 mg, 0.2 mmol), potassium phosphate (100 mg, 0.47 mmol) and [1,1-bis(diphenylphosphino)ferrocene]dichloropalladium (10 mg, 0.01 mmol) were added and the reaction was heated at 80° C. for a further 20 min. The mixture was partitioned between DCM (150 mL) and water (100 mL) and the organic phase was separated then washed with sat. Na₂CO₃ solution (100 mL) and dried (MgSO₄). Removal of the solvent in vacuo followed by trituration of the residue with diethyl ether afforded the title compound: RT=3.67 min, m/z (ES⁺)=432.3 [M+H]⁺.

### Preparation 75

### (S)-4-(5-{4-[tert-Butoxycarbonylamino-3-((S)-2-cyanopyrrolidin-1-yl)-3-oxopropyl]-3-fluorophenyl}pyridine-2-yloxy)piperidine-1-carboxylic acid isopropyl ester

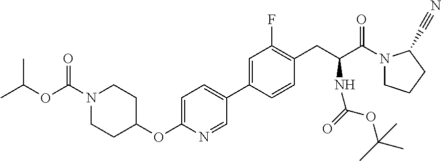

A mixture of 4-[5-(4,4,5,5-tetramethyl-[1,3,2]dioxaborolan-2-yl)pyridin-2-yloxy]piperidine-1-carboxylic acid isopropyl ester (Preparation 17, 239 mg, 0.61 mmol), (S)-trifluoromethanesulfonic acid 4-[2-tert-butoxycarbonylamino-3-((S)-2-cyanopyrrolidin-1-yl)-3-oxopropyl]-3-fluorophenyl ester (Preparation 31, 250 mg, 0.49 mmol), palladium tetrakis (113 mg, 0.1 mmol) and potassium carbonate (169 mg, 1.23 mmol) in toluene (5 mL) were bubbled with argon for 5 min and then heated in a microwave reactor at 75° C. for 30 min. The reaction mixture was diluted with EtOAc and the organics were washed with water then brine, and dried (MgSO₄). Removal of the solvent in vacuo followed by purification by column chromatography (SiO₂, 1H:EtOAc, 75:25, 60:40) afforded the title compound: RT=4.08 min; m/z (ES⁺)=624.6 [M+H]⁺.

### Preparation 76

### (S)-4-{4′-[2-tert-Butoxycarbonylamino-3-((S)-2-cyanopyrrolidin-1-yl)-3-oxopropyl]-3′-fluorobiphenyl-4-yloxy}piperidine-1-carboxylic acid isopropyl ester

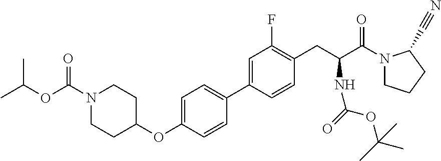

The title compound was prepared from 4-[4-(4,4,5,5-tetramethyl-[1,3,2]dioxaborolan-2-yl)phenoxy]piperidine-1-carboxylic acid isopropyl ester (Preparation 19, 300 mg, 0.77 mmol) and (S)-trifluoromethanesulfonic acid 4-[2-tert-butoxycarbonylamino-3-((S)-2-cyanopyrrolidin-1-yl)-3-oxopropyl]-3-fluorophenyl ester (Preparation 31, 314 mg, 0.62 mmol) employing the procedure outlined in Preparation 38: RT=4.25 min; m/z (ES⁺)=623.6 [M+H]⁺.

### Preparation 77

### (S)-4-{5-[4-(2-tert-Butoxycarbonylamino-3-oxo-3-pyrrolidin-1-ylpropyl)-3-fluorophenyl]pyridin-2-yloxymethyl}piperidine-1-carboxylic acid isopropyl ester

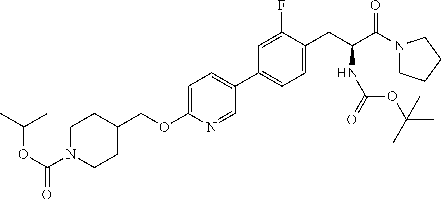

The title compound was prepared from 4-[5-(4,4,5,5-tetramethyl-[1,3,2]dioxaborolan-2-yl)pyridin-2-yloxymethyl]-piperidine-1-carboxylic acid isopropyl ester (Preparation 18, 261 mg, 0.65 mmol) and (S)-trifluoromethanesulfonic acid-4-(2-tert-butoxycarbonylamino-3-oxo-3-pyrrolidin-1-ylpropyl)-3-fluorophenyl ester (Preparation 33, 250 mg, 0.52 mmol) employing the procedure outlined in Preparation 38: RT=4.32 min; m/z (ES⁺)=613.6 [M+H]⁺.

### Preparation 78

### (S)-4-{1-[4′-(2-tert-Butoxycarbonylamino-3-oxo-3-pyrrolidin-1-ylpropyl)-3′-fluorobiphenyl-4-yloxy]ethyl}piperidine-1-carboxylic acid isopropyl ester

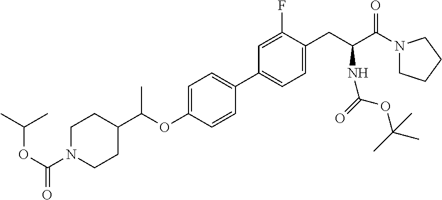

The title compound was prepared from 4-{1-[4-(4,4,5,5-tetramethyl-[1,3,2]dioxaborolan-2-yl)phenoxy]ethyl}piperidine-1-carboxylic acid isopropyl ester (Preparation 22, 107.7 mg, 0.26 mmol) and (S)-trifluoromethanesulfonic acid-4-(2-tert-butoxycarbonylamino-3-oxo-3-pyrrolidin-1-ylpropyl)-3-fluorophenyl ester (Preparation 33, 100 mg, 0.21 mmol) employing the procedure outlined in Preparation 38: RT=4.57 min; m/z (ES⁺)=626.6 [M+H]⁺.

### Preparation 79

### (S)-4-(1-{4′-[2-tert-Butoxycarbonylamino-3-((S)-3-fluoropyrrolidin-1-yl)-3-oxopropyl]-3′-fluorobiphenyl-4-yloxy}ethyl)piperidine-1-carboxylic acid isopropyl ester

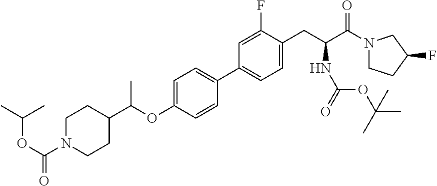

The title compound was prepared from 4-{1-[4-(4,4,5,5-tetramethyl-[1,3,2]dioxaborolan-2-yl)phenoxy]ethyl}piperidine-1-carboxylic acid isopropyl ester (Preparation 22, 260 mg, 0.63 mmol) and (S)-trifluoromethanesulfonic acid 4-[2-tert-butoxycarbonylamino-3-((S)-3-fluoropyrrolidin-1-yl)-3-oxopropyl]3-fluorophenyl ester (Preparation 35, 252 mg, 0.5 mmol) employing the procedure outlined in Preparation 38: RT=4.42 min; m/z (ES⁺)=644.6 [M+H]⁺.

### Preparation 80

### (S)-7-Hydroxy-3,4-dihydro-1H-isoquinoline-2,3-dicarboxylic acid-2-tert-butyl ester

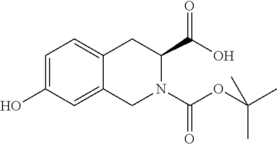

A mixture of (S)-7-hydroxy-1,2,3,4-tetrahydroisoquinoline-3-carboxylic acid (5 g, 25.29 mmol), di-tert-butyl dicarbonate (6.6 g, 30.35 mmol) and triethylamine (3.42 mL, 25.29 mmol) in THF (40 mL) was stirred at r.t. for 1 h. The mixture was partitioned between EtOAc (250 mL) and 1M citric acid solution (500 mL), and the organic phase was separated before being washed with brine (500 mL) and dried (MgSO₄). Removal of the solvent in vacuo followed by trituration with diethyl ether afforded the title compound: RT=3.15 min, m/z (ES⁺)=294.1 [M+H]⁺.

### Preparation 81

### (S)-3-((S)-2-Carbamoylpyrrolidine-1-carbonyl)-7-hydroxy-3,4-dihydro-1H-isoquinoline-2-carboxylic acid tert-butyl ester

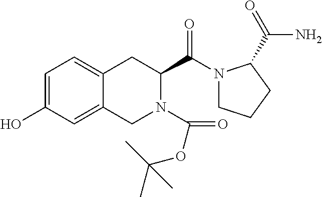

The title compound was prepared by reacting (S)-7-hydroxy-3,4-dihydro-1H-isoquinoline-2,3-dicarboxylic acid-2-tert-butyl ester (Preparation 80, 2.05 g, 7.0 mmol) with L-(−)-prolinamide (1.6 g, 14.0 mmol) employing a procedure similar to that outlined in Preparation 29 but using triethylamine as the base: RT=2.62 min, m/z (ES⁺)=390.3 [M+H]⁺.

### Preparation 82

### (S)-3-((S)-2-Carbamoylpyrrolidine-1-carbonyl)-7-trifluoromethanesulfonyloxy-3,4-dihydro-1H-isoquinoline-2-carboxylic acid tert-butyl ester

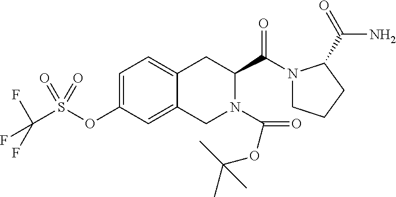

The title compound was prepared by reacting (S)-3-((S)-2-carbamoylpyrrolidine-1-carbonyl)-7-hydroxy-3,4-dihydro-1H-isoquinoline-2-carboxylic acid tert-butyl ester (Preparation 81, 900 mg, 2.31 mmol) with N-phenyltrifluoromethane sulfonimide (909 mg, 2.54 mmol) employing the procedure outlined in Preparation 30: RT=3.41 min, m/z (ES⁺)=522.3 [M+H]⁺.

### Preparation 83

### (S)-3-((S)-2-Cyanopyrrolidine-1-carbonyl)-7-trifluoromethanesulfonyloxy-3,4-dihydro-1H-isoquinoline-2-carboxylic acid tert-butyl ester

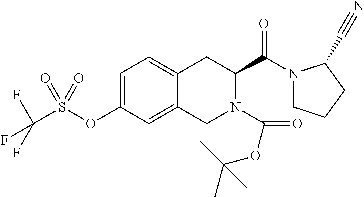

The title compound was prepared by treating (S)-3-((S)-2-carbamoylpyrrolidine-1-carbonyl)-7-trifluoromethanesulfonyloxy-3,4-dihydro-1H-isoquinoline-2-carboxylic acid tert-butyl ester (Preparation 82, 1.2 g, 2.3 mmol) with TFAA (1.62 mL, 11.52 mmol) employing the procedure outlined in Preparation 47: RT=3.93 min, m/z (ES⁺)=504.3 [M+H]⁺.

### Preparation 84

### (S)-3-((S)-2-Cyanopyrrolidine-1-carbonyl)-7-[6-(1-isopropoxycarbonylpiperidin-4-ylmethoxy)pyridin-3-yl]-3,4-dihydro-1H-isoquinoline-2-carboxylic acid tert-butyl ester

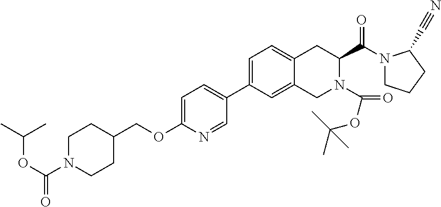

The title compound was prepared by reacting 4-[5-(4,4,5,5-tetramethyl-[1,3,2]dioxaborolan-2-yl)pyridin-2-yloxymethyl]piperidine-1-carboxylic acid isopropyl ester (Preparation 18, 145 mg, 0.36 mmol) with (S)-3-((S)-2-cyanopyrrolidine-1-carbonyl)-7-trifluoromethanesulfonyloxy-3,4-dihydro-1H-isoquinoline-2-carboxylic acid tert-butyl ester (Preparation 83, 151 mg, 0.3 mmol) employing the procedure outlined in Preparation 38: RT=4.32 min, m/z (ES⁺)=632.6 [M+H]⁺.

### Preparation 85

### 4-(4-Bromobenzyloxy)piperidine-1-carboxylic acid isopropyl ester

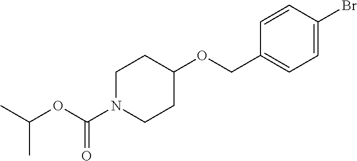

The title compound was prepared by reacting 4-hydroxypiperidine-1-carboxylic acid isopropyl ester (Preparation 2, 2.05 g, 12 mmol) with 4-bromobenzylbromide (3.0 g, 12 mmol) employing the procedure outlined in Preparation 16: RT=4.20 min, m/z (ES⁺)=356.1, 358.1 [M+H]⁺.

### Preparation 86

### 4-[4-(4,4,5,5-Tetramethyl-[1,3,2]dioxaborolan-2-yl)benzyloxy]piperidine-1-carboxylic acid isopropyl ester

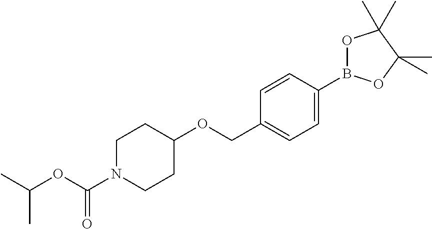

The title compound was prepared from 4-(4-bromobenzyloxy)piperidine-1-carboxylic acid isopropyl ester (Preparation 85, 2.3 g, 6.46 mmol) employing a procedure similar to that outlined in Preparation 26: RT=4.30 min, m/z (ES⁺)=404.4 [M+H]⁺.

### Preparation 87

### (S)-4-[4′-(2-tert-Butoxycarbonylamino-3-oxo-3-pyrrolidin-1-ylpropyl)-3′-fluorobiphenyl-4-ylmethoxy]piperidine-1-carboxylic acid isopropyl ester

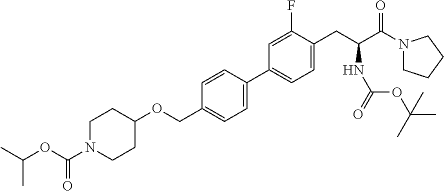

The title compound was prepared by reacting 4-[4-(4,4,5,5-tetramethyl-[1,3,2]dioxaborolan-2-yl)benzyloxy]piperidine-1-carboxylic acid isopropyl ester (Preparation 86, 300 mg, 0.74 mmol) with (S)-trifluoromethanesulfonic acid-4-(2-tert-butoxycarbonylamino-3-oxo-3-pyrrolidin-1-ylpropyl)-3-fluorophenyl ester (Preparation 33, 240 mg, 0.5 mmol) employing a similar procedure to that outlined in Preparation 38: RT=4.43 min, m/z (ES⁺)=612.5 [M+H]⁺.

### Preparation 88

### (S)-4-{4′-[2-tert-Butoxycarbonylamino-3-((S)-3-fluoropyrrolidin-1-yl)-3-oxopropyl]-3′-fluorobiphenyl-4-ylmethoxy}piperidine-1-carboxylic acid isopropyl ester

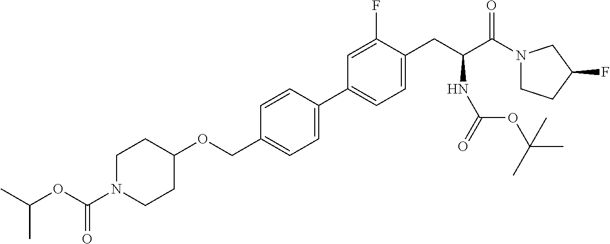

The title compound was prepared by reacting 4-[4-(4,4,5,5-tetramethyl-[1,3,2]dioxaborolan-2-yl)benzyloxy]piperidine-1-carboxylic acid isopropyl ester (Preparation 86, 300 mg, 0.74 mmol) with (S)-trifluoromethanesulfonic acid 4-[2-tert-butoxycarbonylamino-3-((S)-3-fluoropyrrolidin-1-yl)-3-oxopropyl]-3-fluorophenyl ester (Preparation 35, 249 mg, 0.5 mmol) employing a similar procedure to that outlined in Preparation 38: RT=4.20 min, m/z (ES⁺)=630.5 [M+H]⁺.

### Preparation 89

### (E)-3-(4-Benzyloxy-2-fluorophenyl)acrylic acid methyl ester

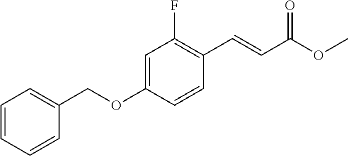

Methyl(triphenylphosphoranylidene)acetate (25.0 g, 74.8 mmol) was added to a solution of 4-benzyloxy-2-fluorobenzaldehyde (9.10 g, 39.5 mmol) in THF (400 mL) and the resulting solution was stirred under reflux conditions for 16 h, before being absorbed onto silica and purified by column chromatography (IH:EtOAc, 3:1) to afford the title compound: RT=4.15 min; m/z (ES⁺)=287.2 [M+H]⁺.

### Preparation 90

### (E)-3-(4-Benzyloxy-2-fluorophenyl)acrylic acid

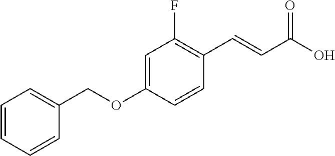

1M NaOH solution (40 mL, 39.8 mmol) was added to a solution of (E)-3-(4-benzyloxy-2-fluorophenyl)acrylic acid methyl ester (Preparation 89, 9.50 g, 33.2 mmol) in MeOH (300 mL) and the resulting suspension was stirred at ambient temperature before heating under reflux conditions for 1 h to afford a solution. The solvent was removed in vacuo and the residue was dissolved in EtOAc (300 mL) and water (600 mL) before adding 1M HCl solution (50 mL) and stirring at ambient temperature for 30 min. The aqueous layer was separated and further extracted with EtOAc (×2). The combined organic layers were washed with brine, dried (MgSO₄), filtered and concentrated in vacuo to afford the title compound: RT=3.72 min; m/z (ES⁺)=562.3 [2M+NH₄]⁺.

### Preparation 91

### (R)-3-[(E)-3-(4-Benzyloxy-2-fluorophenyl)acryloyl]-4-phenyloxazolidin-2-one

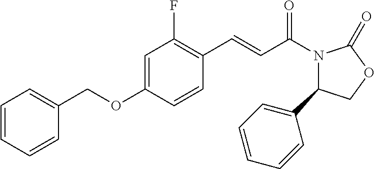

Triethylamine (4.60 mL, 33.0 mmol) and pivaloyl chloride (3.60 mL, 28.2 mmol) were added to a solution of (E)-3-(4-benzyloxy-2-fluorophenyl)acrylic acid (Preparation 90, 6.20 g, 22.8 mmol) in THF (200 mL) at −78° C. and stirred at this temperature for 15 min before stirring at 0° C. for 1 h. In a separate reaction flask, n-butyllithium (1.6M in hexane, 20 mL, 32.0 mmol) was added to a solution of R-(−)-4-phenyl-2-oxazolidinone (5.00 g, 30.6 mmol) in THF (200 mL) at −78° C. and stirred at this temperature for 20 min before adding the above solution, cooled to −78° C., via cannula. The resulting reaction mixture was stirred at −78° C. for 1.5 h and then at ambient temperature for 16 h. The reaction mixture was added to concentrated aqueous NH₄Cl solution (300 mL), then the aqueous layer was separated and further extracted with EtOAc (2×200 mL). The combined organic layers were washed with brine, dried (MgSO₄), and concentrated in vacuo. Recrystallisation (IH:EtOAc, 1:1, 250 mL) afforded the title compound: RT=4.08 min; m/z (ES⁺)=418.2 [M+H]⁺.

### Preparation 92

### (R)-3-[(R)-3-(4-Benzyloxy-2-fluorophenyl)butyryl]-4-phenyloxazolidin-2-one

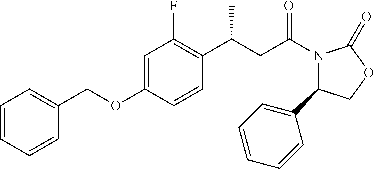

Dimethyl sulfide (30 mL) and methyl magnesium bromide (3.0M solution in Et₂O, 13.0 mL, 39.0 mmol) were added to a suspension of copper(I)bromidedimethyl sulfide (8.80 g, 42.9 mmol) in THF (60 mL) at −40° C. and the resulting reaction mixture was stirred at this temperature for 30 min before warming to −20° C. to −15° C. A solution of (R)-3-[(E)-3-(4-benzyloxy-2-fluorophenyl)acryloyl]-4-phenyloxazolidin-2-one (Preparation 91, 40.0 g, 9.58 mmol) in THF (40 mL) was added dropwise maintaining the temperature between −25° C. and −15° C. and the solution was stirred at this temperature for 2.5 h, then at ambient temperature for 72 h. The reaction was quenched with concentrated aqueous NH₄Cl solution (50 mL) and filtered through celite. The filtrate was diluted with EtOAc, washed with water and brine, dried (MgSO₄), and concentrated in vacuo. Purification by column chromatography (IH:EtOAc, 3:1, 2:1) afforded the title compound: RT=4.35 min; m/z (ES⁺)=434.3 [M+H]⁺.

### Preparation 93

### (R)-3-[(2R,3S)-3-(4-Benzyloxy-5-bromo-2-fluorophenyl)-2-bromobutyryl]-4-phenyloxazolidin-2-one

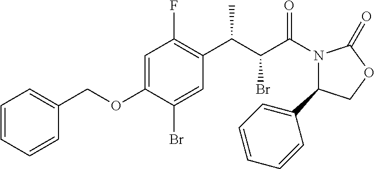

Dibutylborontriflate (10.0 mL, 10.0 mmol) and DIPEA (1.80 mL, 10.3 mmol) were added to a solution of (R)-3-[(R)-3-(4-benzyloxy-2-fluorophenyl)butyryl]-4-phenyloxazolidin-2-one (Preparation 92, 3.00 g, 6.92 mmol) in DCM (50 mL) at −78° C. The resulting reaction mixture was stirred at −78° C. for 10 min then at 0° C. for 1 h. The reaction was cooled to −78° C. and transferred, via cannula, to a solution of N-bromosuccinimide (3.71 g, 20.8 mmol) in DCM (50 mL) under argon, previously cooled to −78° C. The resulting reaction was stirred at −78° C. for 2 h and at 0° C. for 2 h, before being quenched with 0.5M aqueous NaHCO₃ solution. The DCM was removed in vacuo, EtOAc was added and the organic layer was washed with water, brine, dried (MgSO₄), and concentrated in vacuo. Purification by column chromatography (IH:EtOAc, 3:1) afforded the title compound: ¹H NMR δ_{H}(400 MHz, CDCl₃) 7.51-7.30 (m, 11H), 6.68 (m, 1H), 6.12 (m, 1H), 5.26 (m, 1H), 5.13 (s, 2H), 4.59 (m, 1H), 4.20 (m, 1H), 3.63 (m, 1H), 1.52 (m, 3H).

### Preparation 94

### (R)-3-[(2S,3S)-2-Azido-3-(4-benzyloxy-5-bromo-2-fluorophenyl)butyryl]-4-phenyloxazolidin-2-one

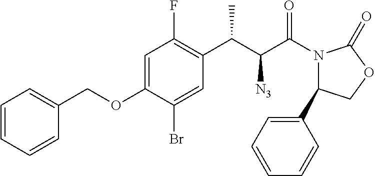

N,N,N′,N′-Tetramethylguanidinium azide (3.70 g, 23.4 mmol) was added to a solution of (R)-3-[(2R,3S)-3-(4-benzyloxy-5-bromo-2-fluorophenyl)-2-bromobutyryl]-4-phenyloxazolidin-2-one (Preparation 93, 3.40 g, 5.75 mmol) in MeCN (25 mL) and the resulting solution was stirred at ambient temperature for 16 h. The reaction mixture was diluted with EtOAc and washed with water and brine, dried (MgSO₄), and concentrated in vacuo. Purification by column chromatography (IH:EtOAc, 2:1) afforded the title compound: RT=4.58 min; m/z (ES⁺)=570.1, 572.1 [M+NH₄]⁺.

### Preparation 95

### (2S,3S)-2-Azido-3-(4-benzyloxy-5-bromo-2-fluorophenyl)butyric acid

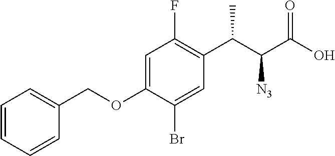

Hydrogen peroxide (35% aqueous solution, 5.00 mL) and Lithium hydroxide monohydrate (810 mg, 19.3 mmol) were added to a solution of (R)-3-[(2S,3S)-2-azido-3-(4-benzyloxy-5-bromo-2-fluorophenyl)butyryl]-4-phenyloxazolidin-2-one (Preparation 94, 3.02 g, 5.46 mmol) in a mixture of THF and water (3:1, 100 mL) at 0° C. and the resulting solution was stirred at this temperature for 6 h. The reaction was quenched with 10% aqueous (w/v) Na₂SO₃ solution and stirred at ambient temperature for 1 h before quenching with water (250 mL) and extracting with EtOAc (4×200 mL). The combined organics were washed with 0.5M HCl solution and brine, dried (MgSO₄), and concentrated in vacuo to afford the title compound: RT=4.03 min; m/z (ES⁺)=425.0, 427.0 [M+NH₄]⁺.

### Preparation 96

### (2S,3S)-3-(2-Fluoro-4-hydroxyphenyl)-2-methylbutyric acid hydrochloride

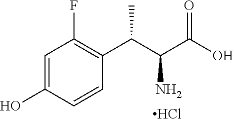

10% Palladium on carbon (1.65 g) was added to a solution of (2S,3S)-2-azido-3-(4-benzyloxy-5-bromo-2-fluorophenyl)butyric acid (Preparation 95, 2.23 g, 5.46 mmol) in a mixture of EtOH and water (9:1, 200 mL) and the resulting reaction mixture was stirred under an atmosphere of hydrogen for 72 h, before filtering through celite. The filtrate was concentrated in vacuo and the remainder dissolved in water and 1M HCl solution, washed with EtOAc and concentrated in vacuo to afford the title compound: RT=1.71 min; m/z (ES⁺)=214.0 [M+H]⁺.

### Preparation 97

### (2S,3S)-2-tert-Butoxycarbonylamino-3-(4-tert-butoxycarbonyloxy-2-fluorophenyl)butyric acid methyl ester

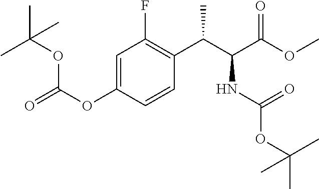

Triethylamine (700 μL, 5.00 mmol) and di-tert-butyldicarboante (1.60 g, 7.33 mmol) were added to a solution of (2S,3S)-3-(2-fluoro-4-hydroxyphenyl)-2-methylbutyric acid hydrochloride (Preparation 96, 682 mg, 2.73 mmol) in a mixture of dioxane and water (19:1, 50 mL) and the resulting solution was stirred for 72 h. The solvent was removed in vacuo and to the residue was added EtOAc (300 mL) and water (100 mL). The mixture was made acidic with 1M HCl solution and stirred vigorously. The aqueous layer was further extracted with EtOAc (2×100 mL) and the combined organic layers were washed with brine, dried (MgSO₄), filtered and concentrated in vacuo. The remainder was dissolved in a mixture of toluene and MeOH (4:1, 50 mL) and cooled to 0° C. before the addition of trimethylsilyldiazomethane (2M in hexane, 2.5 mL, 5.0 mmol). The resulting reaction mixture was stirred from 0° C. to ambient temperature over 30 min, then quenched with AcOH (1 mL) and concentrated in vacuo. Purification by column chromatography (IH:EtOAc, 3:1) afforded the title compound: RT=4.10 min; m/z (ES⁺)=428.2 [M+H]⁺.

### Preparation 98

### (2S,3S)-2-tert-Butoxycarbonylamino-3-(4-tert-butoxycarbonyloxy-2-fluorophenyl)butyric acid

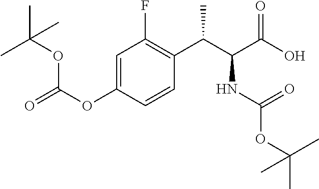

The title compound was prepared from (2S,3S)-2-tert-butoxycarbonylamino-3-(4-tert-butoxycarbonyloxy-2-fluorophenyl)butyric acid methyl ester (Preparation 97, 574 mg, 1.53 mmol) employing the procedure outlined in Preparation 70: ¹H NMR δ_{H}(400 MHz, CD₃OD): 7.35 (m, 1H) 6.95 (m, 2H), 4.43 (m, 1H), 3.49 (m, 1H), 1.55 (s, 9H), 1.36 (m, 12H).

### Preparation 99

### (1S,2S)-Carbonic acid 4-[2-tert-butoxycarbonylamino-3-(3,3-difluoropyrrolidin-1-yl)-1-methyl-3-oxopropyl]-3-fluorophenyl ester tert-butyl ester

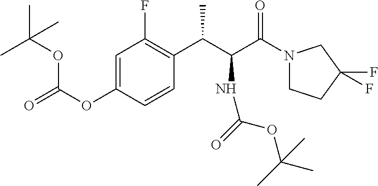

The title compound was prepared by reacting (2S,3S)-2-tert-butoxycarbonylamino-3-(4-tert-butoxycarbonyloxy-2-fluorophenyl)butyric acid (Preparation 98, 318 mg, 0.77 mmol) with 3,3-difluoropyrrolidine hydrochloride (226 mg, 1.57 mmol) employing a similar procedure to that outlined in Preparation 71: RT=4.05 min, m/z (ES⁺)=503.5 [M+H]⁺.

### Preparation 100

### (1S,2S)-[1-(3,3-Difluoropyrrolidine-1-carbonyl)-2-(2-fluoro-4-hydroxyphenyl)propyl]carbamic acid tert-butyl ester

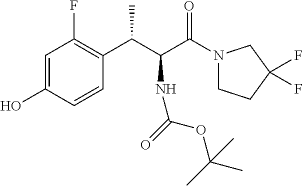

To a solution of (1S,2S)-carbonic acid 4-[2-tert-butoxycarbonylamino-3-(3,3-difluoropyrrolidin-1-yl)-1-methyl-3-oxopropyl]-3-fluorophenyl ester tert-butyl ester (Preparation 99, 232 mg, 0.46 mmol) in DCM (8 mL) was added piperidine (2.0 mL, 20.2 mmol) and the reaction was stirred at r.t. for 16 h. The resulting mixture was partitioned between EtOAc (300 mL) and 1M HCl solution (100 mL) and the organic layer was separated, washed with water then brine, and dried (MgSO₄). Removal of the solvent in vacuo afforded the title compound: R-T=3.40 min, m/z (ES⁺)=403.2 [M+H]⁺.

### Preparation 101

### (1S,2S)-Trifluoromethanesulfonic acid 4-[2-tert-butoxycarbonylamino-3-(3,3-difluoropyrrolidin-1-yl)-1-methyl-3-oxopropyl]-3-fluorophenyl ester

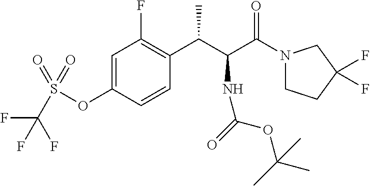

The title compound was prepared by reacting (1S,2S)-[1-(3,3-difluoropyrrolidine-1-carbonyl)-2-(2-fluoro-4-hydroxyphenyl)propyl]carbamic acid tert-butyl ester (Preparation 100, 185 mg, 0.46 mmol) with N-phenyltrifluoromethane sulfonimide (252 mg, 0.71 mmol) employing the procedure outlined in Preparation 30: RT=3.96 min, m/z (ES⁺)=535.4 [M+H]⁺.

### Preparation 102

### 4-(5-{4-[(1S,2S)-2-tert-Butoxycarbonylamino-3-(3,3-difluoropyrrolidin-1-yl)-1-methyl-3-oxopropyl]-3-fluorophenyl}pyridin-2-yloxymethyl)piperidine-1-carboxylic acid isopropyl ester

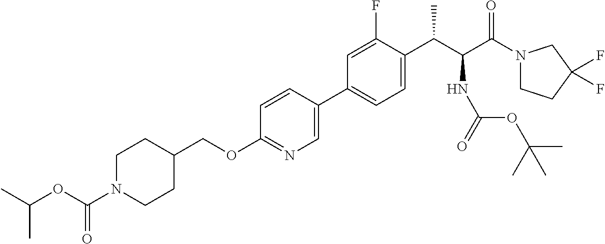

The title compound was prepared by reacting 4-[5-(4,4,5,5-tetramethyl-[1,3,2]dioxaborolan-2-yl)pyridin-2-yloxymethyl]piperidine-1-carboxylic acid isopropyl ester (Preparation 18) with (1S,2S)-trifluoromethanesulfonic acid 4-[2-tert-butoxycarbonylamino-3-(3,3-difluoropyrrolidin-1-yl)-1-methyl-3-oxopropyl]-3-fluorophenyl ester (Preparation 101) employing the procedure outlined in Preparation 61: RT=4.37 min; m/z (ES⁺)=663.5 [M+H]⁺.

### Preparation 103

### (S)-3-((S)-2-Cyanopyrrolidine-1-carbonyl)-7-{6-[1-(1-isopropoxycarbonylpiperidin-4-yl)ethoxy]pyridin-3-yl}-3,4-dihydro-1H-isoquinoline-2-carboxylic acid tert-butyl ester

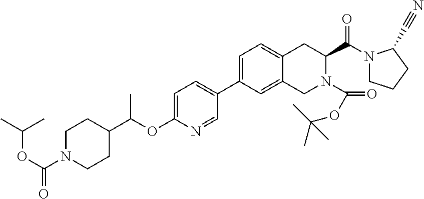

The title compound was prepared by reacting 4-{1-[5-(4,4,5,5-tetramethyl-[1,3,2]dioxaborolan-2-yl)pyridin-2-yloxy]ethyl}piperidine-1-carboxylic acid isopropyl ester (Preparation 21, 150 mg, 0.36 mmol) with (S)-3-((S)-2-cyanopyrrolidine-1-carbonyl)-7-trifluoromethanesulfonyloxy-3,4-dihydro-1H-isoquinoline-2-carboxylic acid tert-butyl ester (Preparation 83, 156 mg, 0.3 mmol) employing the procedure outlined in Preparation 38: RT=4.39 min, m/z (ES⁺)=646.6 [M+H]⁺.

### Preparation 104

### (S)-3-((S)-2-Cyanopyrrolidine-1-carbonyl)-7-{6-[(S)-1-(1-isopropoxycarbonylpiperidin-4-yl)ethoxy]pyridin-3-yl}-3,4-dihydro-1H-isoquinoline-2-carboxylic acid tert-butyl ester

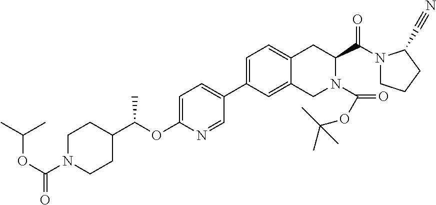

The title compound was afforded via chiral HPLC separation of (S)-3-((S)-2-cyanopyrrolidine-1-carbonyl)-7-{6-[1-(1-isopropoxycarbonylpiperidin-4-yl)ethoxy]pyridin-3-yl}-3,4-dihydro-1H-isoquinoline-2-carboxylic acid tert-butyl ester (Preparation 103): Daicel chiral pack IA 250×20 mm, MTBE:EtOH:DEA, 40:60:0.1, 10 mL/min, 285 nm.

### Preparation 105

### (S)-3-((S)-2-Cyanopyrrolidine-1-carbonyl)-7-{6-[(R)-1-(1-isopropoxycarbonylpiperidin-4-yl)ethoxy]pyridin-3-yl}-3,4-dihydro-1H-isoquinoline-2-carboxylic acid tert-butyl ester

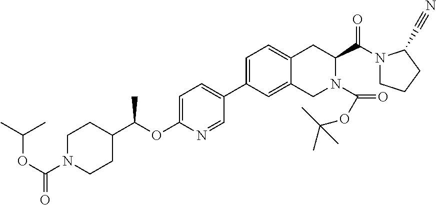

The title compound was afforded via chiral HPLC separation of (S)-3-((S)-2-cyanopyrrolidine-1-carbonyl)-7-{6-[1-(1-isopropoxycarbonylpiperidin-4-yl)ethoxy]pyridin-3-yl}-3,4-dihydro-1H-isoquinoline-2-carboxylic acid tert-butyl ester (Preparation 103): Daicel chiral pack IA 250×20 mm, MTBE:EtOH:DEA, 40:60:0.1, 10 mL/min, 285 nm.

### Preparation 106

### (S)-4-(4{4-[2-tert-Butoxycarbonylamino-3-((S)-3-fluoropyrrolidin-1-yl)-3-oxopropyl]-3-fluorophenyl}-pyridin-2-yloxymethyl)piperidine-1-carboxylic acid isopropyl ester

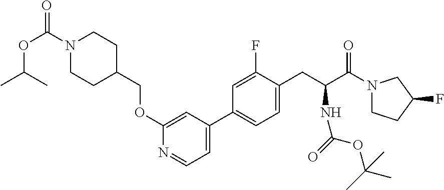

A solution of 4-[4-(4,4,5,5-tetramethyl-[1,3,2]dioxaborolan-2-yl)pyridin-2-yloxymethyl]piperidine-1-carboxylic acid isopropyl ester (Preparation 23, 910 mg, 2.25 mmol) and potassium hydrogen fluoride (878 mg, 11.24 mmol) in MeOH (9 mL) was stirred at r.t. for 16 h. The solvent was removed in vacuo then the residue was washed with diethyl ether and recrystallised from MeCN to afford a white solid. The material was combined with (S)-trifluoromethanesulfonic acid 4-[2-tert-butoxycarbonylamino-3-((S)-3-fluoropyrrolidin-1-yl)-3-oxopropyl]3-fluorophenyl ester (Preparation 35, 588 mg, 1.17 mmol), palladium (II) acetate (26 mg, 1.17 mmol) and potassium carbonate (462 mg, 3.35 mmol) in a mixture of toluene (5 mL) and water (1 mL), and the reaction was heated to 110° C. for 24 h. After cooling to r.t. the reaction mixture was extracted with DCM (3×10 mL) and the organic fractions were combined, dried (MgSO₄), and the solvent removed in vacuo. Purification by column chromatography (SiO₂, IH:EtOAc, 7:3, 1:1) afforded the title compound: RT=4.20 min; m/z (ES⁺)=631.4 [M+H]⁺.

### Example 1

### (S)-4-(5-{4-[2-Amino-3-(3,3-difluoropyrrolidin-1-yl)-3-oxopropyl]-3-fluorophenyl}pyridine-2-yloxy)piperidine-1-carboxylic acid isopropyl ester

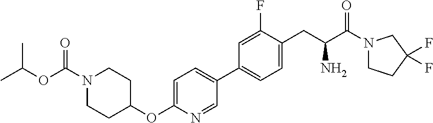

A solution of (S)-4-(5-{4-[2-tert-butoxycarbonylamino-3-(3,3-difluoropyrrolidin-1-yl)-3-oxopropyl]-3-fluorophenyl}pyridin-2-yloxy)piperidine-1-carboxylic acid isopropyl ester (Preparation 38, 280 mg, 0.44 mmol) in DCM (5 mL) under argon was cooled to 0° C. TFA (1 mL) was added and the reaction stirred at 0° C. for 2 h. A further portion of TFA (0.5 mL) was added and stiffing continued for 1 h. The reaction was quenched with saturated NaHCO₃ solution and organics were extracted into DCM. The organic phase was washed with brine, dried (MgSO₄) and the solvent was removed in vacuo. Purification by column chromatography (SiO₂, DCM:MeOH, 98:2, 97:3, 95:5, 90:10, 80:20) afforded the title compound: RT=3.18 min; m/z (ES⁺)=535.3 [M+H]⁺.

The following examples were prepared by treating the appropriate tert-butyl carbamate protected amine with TFA employing a procedure similar to that outlined in Example 1:

| [symbol] x | Structure | Name | LCMS |
|---|---|---|---|
| [symbol] | 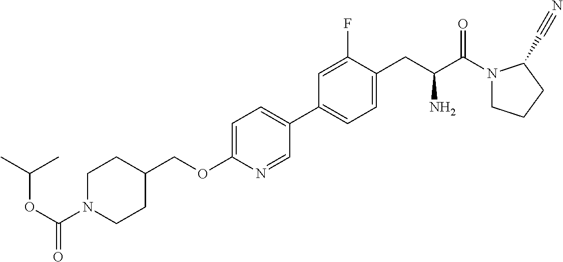 | (S)-4-(5-{4-[2-Amino-3-((S)-2- cyanopyrrolidin-1-yl)-3- oxopropyl]-3-fluorophenyl}- pyridin-2-yloxymethyl)- piperidine-1-carboxylic acid isopropyl ester | RT = 2.92 min; m/z (ES⁺) = 538.5 [M + H]⁺ |
| [symbol] | 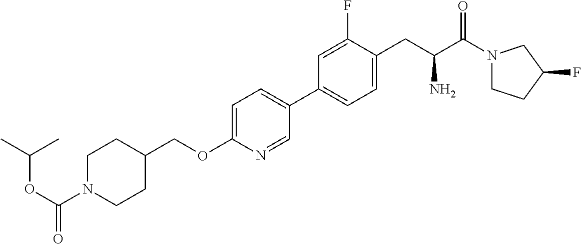 | (S)-4-(5-{4-[2-Amino-3-((S)-3- fluoropyrrolidin-1-yl)-3- oxopropyl]-3-fluorophenyl}- pyridin-2-yloxymethyl)- piperidine-1-carboxylic acid isopropyl ester | RT = 2.95 min; m/z (ES⁺) = 531.4 [M + H]⁺ |
| [symbol] | 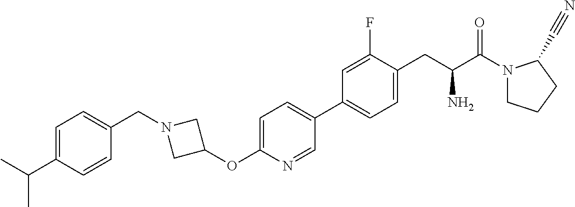 | (S)-1-[2-Amino-3-(2-fluoro-4- {6-[1-(4-isopropylbenzyl)- azetidin-3-yloxy]pyridin-3- yl}phenyl)propionyl]pyrrolidine- 2-carbonitrile | RT = 2.57 min; m/z (ES⁺) = 542.5 [M + H]⁺ |
| [symbol] | 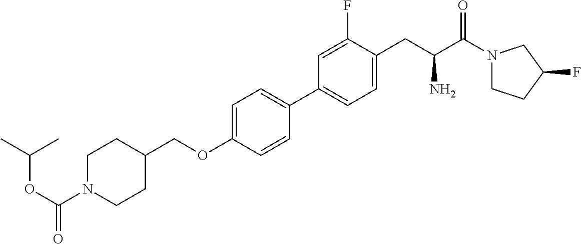 | (S)-4-{4′-[2-Amino-3-((S)-3- fluoropyrrolidin-1-yl)-3- oxopropyl]-3′-fluorobiphenyl-4- yloxymethyl}piperidine-1- carboxylic acid isopropyl ester | RT = 2.92 min; m/z (ES⁺) = 530.4 [M + H]⁺ |
| [symbol] | 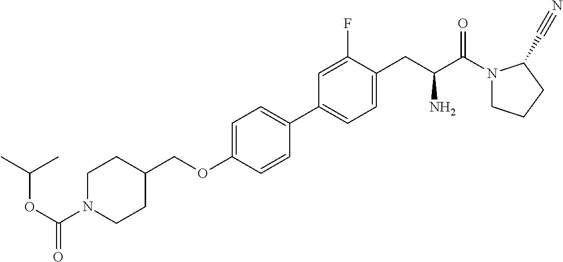 | (S)-4-{4′-[2-Amino-3-((S)-2- cyanopyrrolidin-1-yl)-3- oxopropyl]-3′-fluorobiphenyl-4- yloxymethyl}piperidine-1- carboxylic acid isopropyl ester | RT = 2.97 min; m/z (ES⁺) = 537.4 [M + H]⁺ |
| [symbol] | 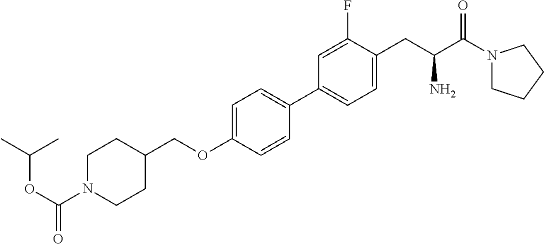 | (S)-4-[4′-(2-Amino-3-oxo-3- pyrrolidin-1-yl-propyl)-3′- fluorobiphenyl-4-yloxymethyl}- piperidine-1-carboxylic acid isopropyl ester | RT = 3.03 min; m/z (ES⁺) = 512.4 [M + H]⁺ |
| [symbol] | 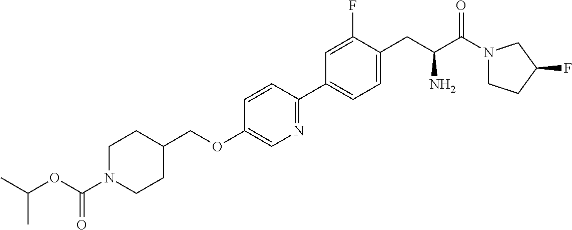 | (S)-4-(6-{4-[2-Amino-3-((S)-3- fluoropyrrolidin-1-yl)-3- oxopropyl]-3-fluorophenyl}- pyridin-3-yloxymethyl}- piperidine-1-carboxylic acid isopropyl ester | RT = 2.80 min; m/z (ES⁺) = 531.4 [M + H]⁺ |
| [symbol] | 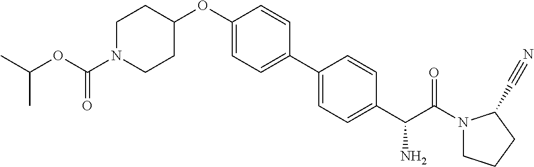 | (R)-4-{4′-[1-Amino-2-((S)-2- cyanopyrrolidin-1-yl)-2- oxoethyl]biphenyl-4-yloxy}- piperidine-1-carboxylic acid isopropyl ester | RT = 2.88 min; m/z (ES⁺) = 491.4 [M + H]⁺ |
| [symbol] | 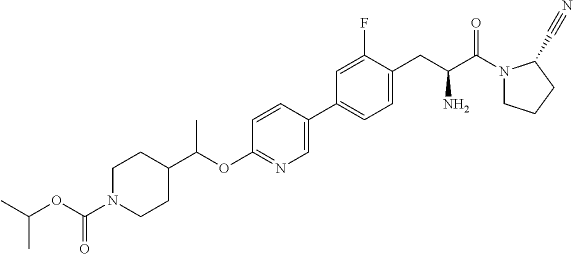 | (S)-4-[1-(5-{4-[2-Amino-3-((S)- 2-cyanopyrrolidin-1-yl)-3- oxopropyl]-3-fluorophenyl}- pyridin-2-yloxy)ethyl]- piperidine-1-carboxylic acid isopropyl ester | RT = 2.98 min; m/z (ES⁺) = 552.5 [M + H]⁺ |
| [symbol] | 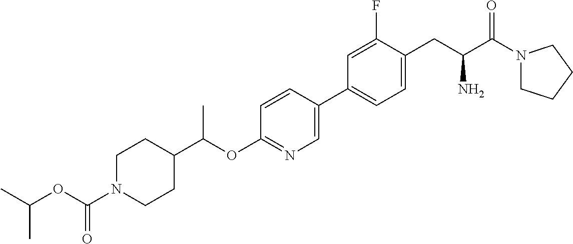 | (S)-4-(1-{5-[4-(2-Amino-3-oxo- 3-pyrrolidin-1-ylpropyl)-3- fluorophenyl]pyridin-2- yloxy}ethyl)piperidine-1- carboxylic acid isopropyl ester | RT = 2.93 min; m/z (ES⁺) = 527.5 [M + H]⁺ |
| [symbol] | 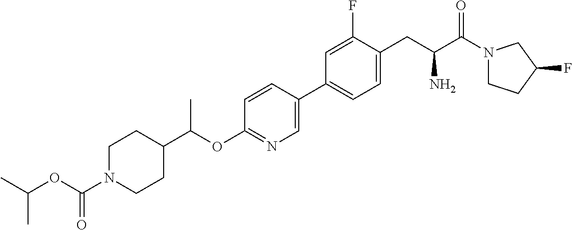 | (S)-4-[1-(5-{4-[2-Amino-3-((S)- 3-fluoropyrrolidin-1-yl)-3- oxopropyl]-3-fluorophenyl}- pyridin-2-yloxy)ethyl]- piperidine-1-carboxylic acid isopropyl ester | RT = 2.92 min; m/z (ES⁺) = 545.5 [M + H]⁺ |
| [symbol] | 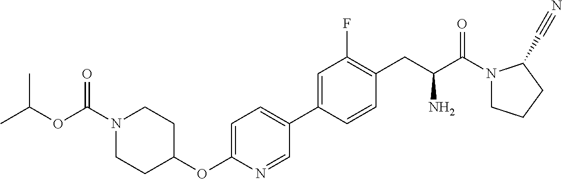 | (S)-4-(5-{4-[2-Amino-3-((S)-2- cyanopyrrolidin-1-yl)-3- oxopropyl]-3-fluorophenyl}- pyridin-2-yloxy)piperidine-1- carboxylic acid isopropyl ester | RT = 2.96 min; m/z (ES⁺) = 524.5 [M + H]⁺ |
| [symbol] | 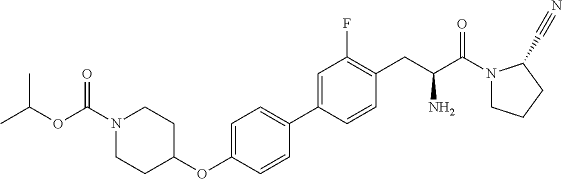 | (S)-4-{4′-[2-Amino-3-((S)-2- cyanopyrrolidin-1-yl)-3- oxopropyl]-3′-fluorobiphenyl-4- yloxy}piperidine-1-carboxylic acid isopropyl ester | RT = 2.91 min; m/z (ES⁺) = 523.4 [M + H]⁺ |
| [symbol] | 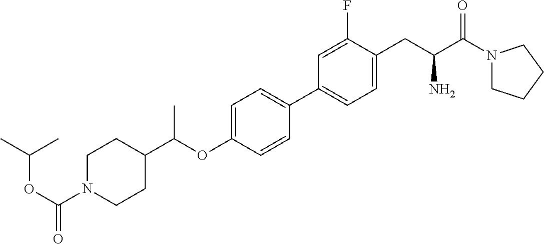 | (S)-4-{1-[4′-(2-Amino-3-oxo-3- pyrrolidin-1-ylpropyl)-3′- fluorobiphenyl-4-yloxy]- ethyl}piperidine-1-carboxylic acid isopropyl ester | RT = 3.12 min; m/z (ES⁺) = 526.5 [M + H]⁺ |
| [symbol] | 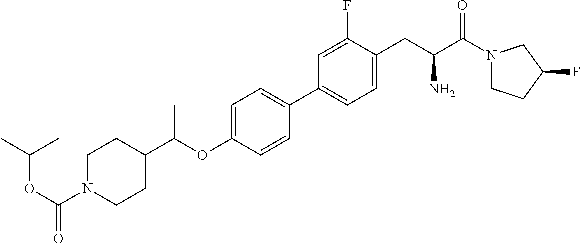 | (S)-4-(1-{4′-[2-Amino-3-((S)-3- fluoropyrrolidin-1-yl)-3- oxopropyl]-3′-fluorobiphenyl-4- yloxy}ethyl)piperidine-1- carboxylic acid isopropyl ester | RT = 3.00 min; m/z (ES⁺) = 544.5 [M + H]⁺ |

[symbol]

indicates data missing or illegible when filed

### Example 17

### (S)-4-(5-{4-[2-Amino-3-((S)-3-fluoropyrrolidin-1-yl)-3-oxopropyl]-3-fluorophenyl}pyridin-2-yloxy)piperidine-1-carboxylic acid isopropyl ester hydrochloride

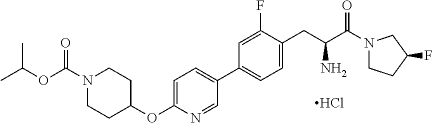

A solution of (S)-4-(5-{4-[2-tert-butoxycarbonylamino-3-((S)-3-fluoropyrrolidin-1-yl)-3-oxopropyl]-3-fluorophenyl}pyridin-2-yloxy)piperidine-1-carboxylic acid isopropyl ester (Preparation 39, 230 mg, 0.37 mmol) in DCM (4 mL) under argon was cooled to 0° C. TFA (1 mL) was added and the reaction was stirred for 16 h. The mixture was diluted with DCM and a saturated Na₂CO₃ solution was added to adjust the pH. The organic phase was passed through a phase separator and the solvent was removed in vacuo. Purification by column chromatography (SiO₂, DCM:MeOH, 100:0, 95:5, 93:7) afforded the title compound as its free base. The product was taken into a solution of 4M HCl in dioxane and stirred at r.t. for 15 min. Removal of the solvent in vacuo afforded the title compound. RT=2.81 min; m/z (ES⁺)=517.4 [M+H]⁺.

### Example 18

### (S)-2-Amino-3-(2-fluoro-4-{6-[1-(4-isopropylbenzypazetidin-3-yloxy]pyridin-3-yl}phenyl)-1-pyrrolidin-1-yl propan-1-one p-toluenesulfonic acid salt

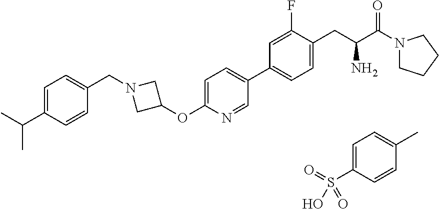

A solution of (S)-[1-(2-fluoro-4-{6-[1-(4-isopropylbenzyl)azetidin-3-yloxy]pyridin-3-yl}benzyl)-2-oxo-2-pyrrolidin-1-yl ethyl]carbamic acid tert-butyl ester (Preparation 59, 75 mg, 0.12 mmol) in DCM (4 mL) under argon was cooled to 0° C. TFA (1 mL) was added and the reaction was stirred for 3 h. The mixture was diluted with DCM and a saturated NaHCO₃ solution was added to adjust the pH. The organic phase was separated, dried (MgSO₄) and the solvent was removed in vacuo. Purification by column chromatography (SiO₂, DCM:MeOH, 100:0, 98:2, 95:5, 90:10) afforded the title compound as the free amine. The product was dissolved in DCM and p-toluenesulfonic acid monohydrate (1 eq, 15.2 mg, 0.08 mmol) was added as a solution in MeOH. The mixture was stirred for 15 min. Removal of the solvent in vacuo afforded the title compound: RT=2.57 min; m/z (ES⁺)=517.5 [M+H]⁺.

The following examples were prepared by treating the appropriate tert-butyl carbamate protected amine with TFA employing a procedure similar to that outlined in Example 18.

| Ex | Structure | Name | LCMS |
|---|---|---|---|
| 19 | 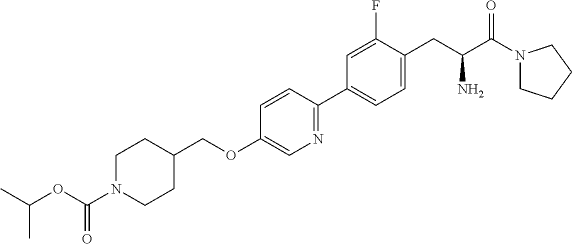 | (S)-4-{6-[4-(2-Amino-3- oxo-3-pyrrolidin-1-yl propyl)-3-fluoro- phenyl]pyridin-3- yloxymethyl}piperidine- 1-carboxylic acid isopropyl ester | RT = 2.85 min; m/z (ES⁺) = 513.5 [M + H]⁺ |
| 20 | 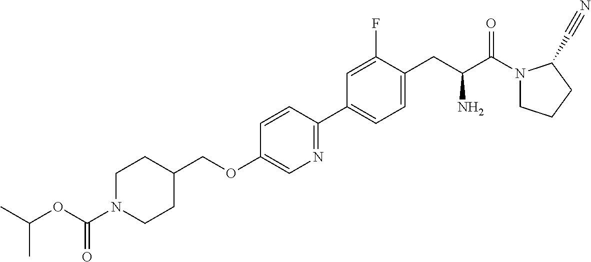 | (S)-4-(6-{4-[2-Amino-3- ((S)-2-cyanopyrrolidin- 1-yl)-3-oxopropyl]-3- fluorophenyl}pyridin-3- yloxymethyl)piperidine- 1-carboxylic acid isopropyl ester | RT = 2.90 min; m/z (ES⁺) = 538.5 [M + H]⁺ |

### Example 21

### (S)-4-{4′-[1-Amino-2-((S)-3-fluoropyrrolidin-1-yl)-2-oxoethyl]biphenyl-4-yloxy}piperidine-1-carboxylic acid isopropyl ester

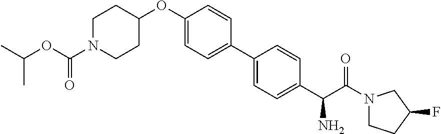

A solution of 4-{4′-[1-tert-butoxycarbonylamino-2-((S)-3-fluoropyrrolidin-1-yl)-2-oxoethyl]biphenyl-4-yloxy}piperidine-1-carboxylic acid isopropyl ester (Preparation 44, 106 mg, 0.18 mmol) in DCM (5 mL) under argon, was cooled to 0° C. TFA (1 mL) was added and the reaction was stirred at 0° C. for 1.5 h. The reaction was quenched by adding sat. Na₂CO₃ solution (30 mL) and organics were extracted into EtOAc (50 mL). The organic layer was washed with brine (50 mL) then dried (MgSO₄). Removal of the solvent in vacuo afforded the title compound: RT=2.85 min; m/z (ES⁺)=484.5 [M+H]⁺.

### Example 22

### 4-[(R)-1-(5-{4-[(S)-2-Amino-3-((S)-2-cyanopyrrolidin-1-yl)-3-oxopropyl]-3-fluorophenyl}pyridin-2-yloxy)ethyl]piperidine-1-carboxylic acid isopropyl ester

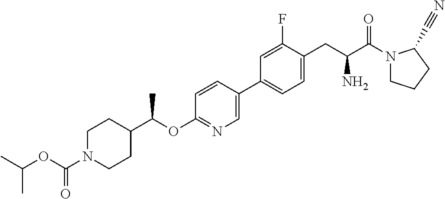

The title compound was afforded via chiral HPLC separation of (S)-4-[1-(5-{4-[2-amino-3-((S)-2-cyanopyrrolidin-1-yl)-3-oxopropyl]-3-fluorophenyl}pyridin-2-yloxy)ethyl]piperidine-1-carboxylic acid isopropyl ester (Example 10): Daicel chiral pack IA 250×20 mm, MeCN:MeOH:DEA, 25:75:0.1, 15 mL/min, 265 nm.

The following examples were afforded via chiral HPLC purification of the relevant mixture of diastereoisomers, employing procedures similar to that outlined in Example 22.

| Ex | Structure | Name | LCMS |
|---|---|---|---|
| 23 | 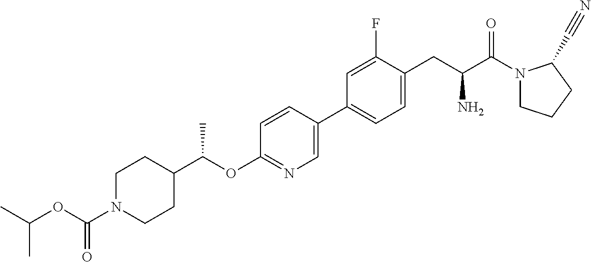 | 4-[(S)-1-(5-{4-[(S)-2- Amino-3-((S)-2- cyanopyrrolidin-1-yl)- 3-oxopropyl]-3- fluorophenyl}pyridin- 2-yloxy)ethyl]- piperidine-1- carboxylic acid isopropyl ester | RT = 2.98 min; m/z (ES⁺) = 552.5 [M + H]⁺ |
| 24 | 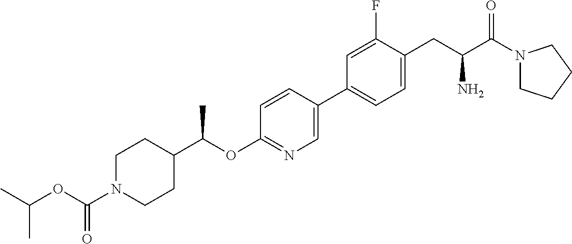 | 4-((R)-1-{5-[4-((S)-2- Amino-3-oxo-3- pyrrolidin-1-ylpropyl)- 3-fluorophenyl]- pyridin-2- yloxy}ethyl)- piperidine-1- carboxylic acid isopropyl ester | RT = 2.93 min; m/z (ES⁺) = 527.5 [M + H]⁺ |
| 25 | 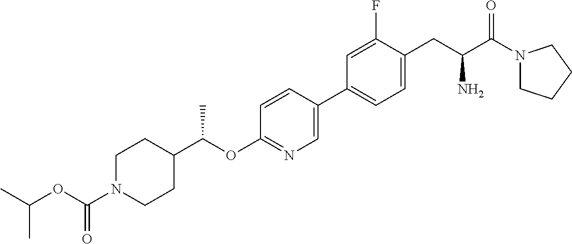 | 4-((S)-1-{5-[4-((S)-2- Amino-3-oxo-3- pyrrolidin-1-ylpropyl)- 3-fluorophenyl]- pyridin-2-yloxy}- ethyl)piperidine-1- carboxylic acid isopropyl ester | RT = 2.93 min; m/z (ES⁺) = 527.5 [M + H]⁺ |
| 26 | 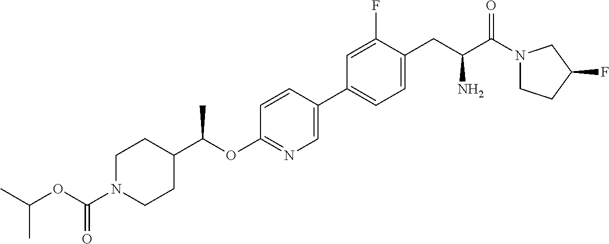 | 4-[(R)-1-(5-{4-[(S)-2- Amino-3-((S)-3- fluoropyrrolidin-1-yl)- 3-oxopropyl]-3-fluoro- phenyl} pyridin-2- yloxy)ethyl]piperidine- 1-carboxylic acid isopropyl ester | RT = 2.92 min; m/z (ES⁺) = 545.5 [M + H]⁺ |
| 27 | 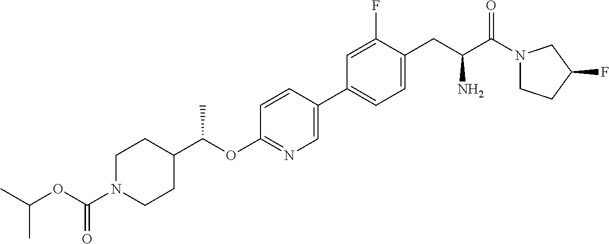 | 4-[(S)-1-(5-{4-[(S)-2- Amino-3-((S)-3- fluoropyrrolidin-1-yl)- 3-oxopropyl]-3-fluoro- phenyl}pyridin-2- yloxy)ethyl]piperidine- 1-carboxylic acid isopropyl ester | RT = 2.92 min; m/z (ES⁺) = 545.5 [M + H]⁺ |

### Example 28

### (S)-4-{4′-[2-Amino-3-((S)-2-cyanopyrrolidin-1-yl)-3-oxopropyl]biphenyl-4-yloxymethyl}piperidine-1-carboxylic acid isopropyl ester hydrochloride

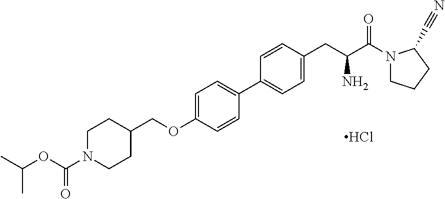

To a solution of (S)-4-{4′-[2-tert-butoxycarbonylamino-3-((S)-2-cyanopyrrolidin-1-yl)-3-oxopropyl]biphenyl-4-yloxymethyl}piperidine-1-carboxylic acid isopropyl ester (Preparation 73, 56 mg, 0.09 mmol) in DCM was added TFA (2.5 mL). After stiffing at r.t. for 2.5 h the reaction mixture was concentrated and the residue was partitioned between EtOAc (100 mL) and sat. NaHCO₃ solution (100 mL). The layers were separated and the aqueous phase was extracted with EtOAc (3×50 mL). The combined organic fractions were dried (MgSO₄), filtered and concentrated in vacuo. Purification of the residue by column chromatography (SiO₂, DCM:MeOH, 100:7.5) afforded the title compound as the free amine. The product was dissolved in MeOH (50 mL) and treated with 1M HCl solution (1 mL). Removal of the solvent in vacuo followed by co-distillation with MeOH (2×50 mL) afforded the title compound: RT=2.97 min; m/z (ES⁺)=519.5 [M+H]⁺.

### Example 29

### (S)-4-{4′-[2-Amino-3-((S)-3-fluoropyrrolidin-1-yl)-3-oxopropyl]biphenyl-4-yloxymethyl}piperidine-1-carboxylic acid isopropyl ester hydrochloride

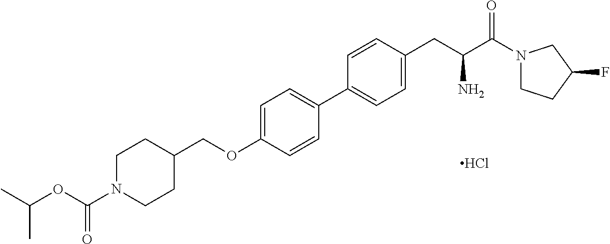

The title compound was prepared from (S)-4-{4′-[2-tert-butoxycarbonylamino-3-((S)-3-fluoropyrrolidin-1-yl)-3-oxopropyl]biphenyl-4-yloxymethyl}piperidine-1-carboxylic acid isopropyl ester (Preparation 71, 75 mg, 0.12 mmol) employing the procedure outlined in Example 28: RT=3.03 min; m/z (ES⁺)=512.4 [M+H]⁺.

### Example 30

### 2-Amino-3-(2-fluoro-4-{6-[1-(6-methylpyrazin-2-yl)piperidin-4-ylmethoxy]pyridin-3-yl}phenyl)-1-pyrrolidin-1-yl-propan-1-one

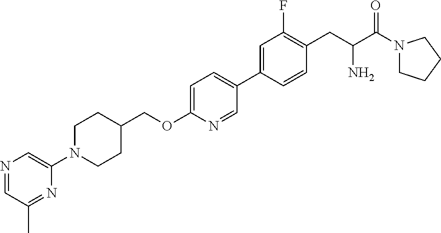

A solution of [1-(6-methylpyrazin-2-yl)piperidin-4-yl]methanol (144 mg, 0.7 mmol) and potassium tert-butoxide (67 mg, 0.7 mmol) in THF (3 mL) was stirred for 5 min before adding (S)-{1-[2-fluoro-4-(6-fluoropyridin-3-yl)benzyl]-2-oxo-2-pyrrolidin-1-ylethyl}carbamic acid tert-butyl ester (Preparation 74, 75 mg, 0.17 mmol) and heating in a microwave reactor at 150° C. for 30 min. The reaction mixture was filtered through a plug of MgSO₄ and solvent was removed in vacuo. Purification of the residue by preparative HPLC afforded the title compound: RT=2.82 min; m/z (ES⁺)=519.5 [M+H]⁺.

The following examples were prepared by treating (S)-{1-[2-fluoro-4-(6-fluoropyridin-3-yl)benzyl]-2-oxo-2-pyrrolidin-1-ylethyl}carbamic acid tert-butyl ester (Preparation 74) with the appropriate alcohol employing the procedure outlined in Example 30.

| [symbol] x | Structure | Name | LCMS |
|---|---|---|---|
| [symbol] | 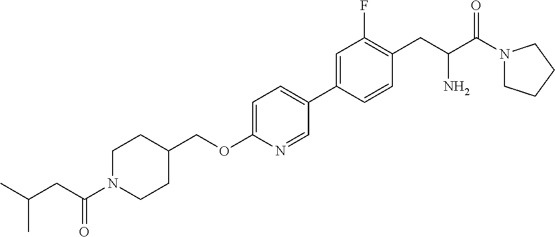 | 1-(4-{5-[4-(2-Amino-3-oxo-3- pyrrolidin-1-ylpropyl)-3- fluorophenyl]pyridin-2- yloxymethyl}piperidin-1-yl)-3- methylbutan-1-one | RT = 2.77 min; m/z (ES⁺) = 511.5 [M + H]⁺ |
| [symbol] | 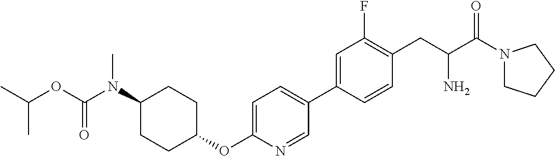 | (4-{5-[4-(2-Amino-3-oxo-3- pyrrolidin-1-ylpropyl)-3- fluorophenyl]pyridin-2- yloxy}cyclohexyl)methylcarbamic acid isopropyl ester | RT = 3.00 min; m/z (ES⁺) = 527.5 [M + H]⁺ |
| [symbol] | 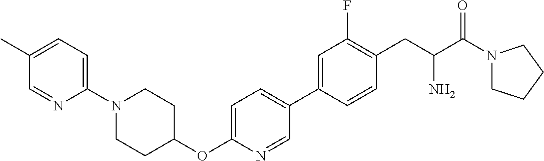 | 2-Amino-3-{2-fluoro-4-[6-(5′- methyl-3,4,5,6-tetrahydro-2H- [1,2′]bipyridinyl-4-yloxy)pyridin- 3-yl]phenyl]phenyl}-1-pyrrolidin- 1-ylpropan-1-one | RT = 2.27 min; m/z (ES⁺) = 504.5 [M + H]⁺ |

[symbol]

indicates data missing or illegible when filed

### Example 34

### (S)-2-Amino-3-(2-fluoro-4-{6-[1-(3-isopropyl-[1,2,4]oxadiazol-5-yl)piperidin-4-ylmethoxy]pyridine-3-yl}phenyl)-1-pyrrolidin-1-yl-propan-1-one

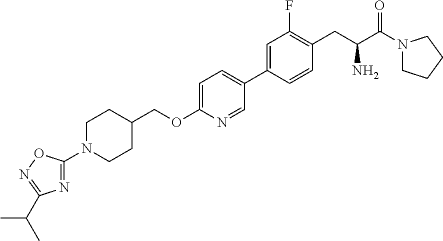

The title compound was prepared by reacting [1-(3-isopropyl-[1,2,4]oxadiazol-5-yl)piperidin-4-yl]methanol (157 mg, 0.7 mmol) with (S)-{1-[2-fluoro-4-(6-fluoropyridin-3-yl)benzyl]-2-oxo-2-pyrrolidin-1-ylethyl}carbamic acid tert-butyl ester (Preparation 74, 75 mg, 0.17 mmol) employing the procedure outlined in Example 30. Further purification by chiral HPLC afforded the title compound: RT=2.77 min; m/z (ES⁺)=537.4 [M+H]⁺.

### Example 35

### (S)-1-(4-{5-[4-(2-Amino-3-oxo-3-pyrrolidin-1-ylpropyl)-3-fluorophenyl]pyridin-2-yloxymethyl}piperidin-1-yl)-3-methylbutan-1-one

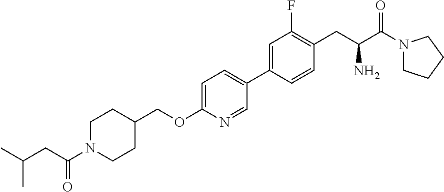

The title compound was prepared by reacting 1-(4-hydroxymethylpiperidin-1-yl)-3-methyl butan-1-one (49 mg, 0.24 mmol) with (S)-{1-[2-fluoro-4-(6-fluoropyridin-3-yl)benzyl]-2-oxo-2-pyrrolidin-1-ylethyl}carbamic acid tert-butyl ester (Preparation 74, 70 mg, 0.16 mmol) employing the procedure outlined in Example 30. Further purification by chiral HPLC afforded the title compound: RT=2.77 min; m/z (ES⁺)=511.5 [M+H]⁺.

### Example 36

### (S)-4-{5-[4-(2-Amino-3-oxo-3-pyrrolidin-1-ylpropyl)-3-fluorophenyl]pyridine-2-yloxymethyl}piperidine-1-carboxylic acid isopropyl ester

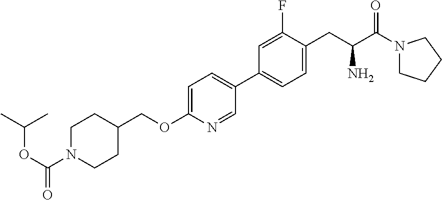

The title compound was prepared from (S)-4-{5-[4-(2-tert-butoxycarbonylamino-3-oxo-3-pyrrolidin-1-ylpropyl)-3-fluorophenyl]pyridin-2-yloxymethyl}piperidine-1-carboxylic acid isopropyl ester (Preparation 77) employing the procedure outlined in Example 21: RT=2.96 min; m/z (ES⁺)=513.4 [M+H]⁺.

### Example 37

### 4-{(S)-1-[4% ((S)-2-Amino-3-oxo-3-pyrrolidin-1-ylpropyl)-3′-fluorobiphenyl-4-yloxy]ethyl}piperidine-1-carboxylic acid isopropyl ester

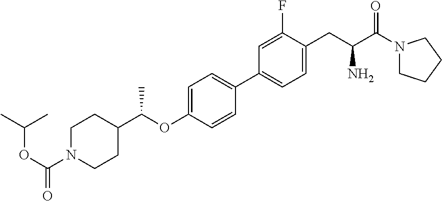

The title compound was afforded via chiral HPLC separation of (S)-4-{1-[4′-(2-amino-3-oxo-3-pyrrolidin-1-ylpropyl)-3′-fluorobiphenyl-4-yloxy]ethyl}piperidine-1-carboxylic acid isopropyl ester (Example 15): Daicel chiral pack IA 250×20 mm, MeCN:MeOH:THF:DEA, 50:50:2:0.1, 15 mL/min, 285 nm.

### Example 38

### 4-{(R)-1-[4′-((S)-2-Amino-3-oxo-3-pyrrolidin-1-ylpropyl)-3′-fluorobiphenyl-4-yloxy]ethyl}piperidine-1-carboxylic acid isopropyl ester

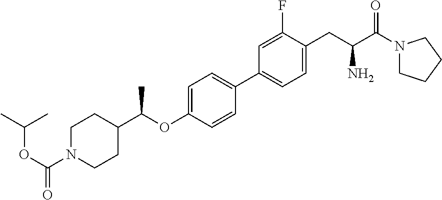

The title compound was afforded via chiral HPLC separation of (S)-4-{1-[4′-(2-amino-3-oxo-3-pyrrolidin-1-ylpropyl)-3′-fluorobiphenyl-4-yloxy]ethyl}piperidine-1-carboxylic acid isopropyl ester (Example 15): Daicel chiral pack IA 250×20 mm, MeCN:MeOH:THF:DEA, 50:50:2:0.1, 15 mL/min, 285 nm.

### Example 39

### 4-((R)-1-{4′-[(S)-2-Amino-3-((S)-3-fluoropyrrolidin-1-yl)-3-oxopropyl]-3′-fluorobiphenyl-4-yloxy}ethyl)piperidine-1-carboxylic acid isopropyl ester

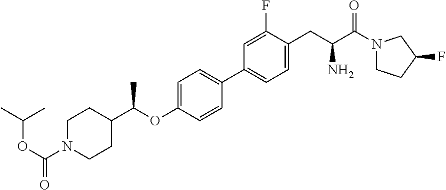

The title compound was afforded via chiral HPLC separation of (S)-4-(1-{4′-[2-amino-3-((S)-3-fluoropyrrolidin-1-yl)-3-oxopropyl]-3′-fluorobiphenyl-4-yloxy}ethyl)piperidine-1-carboxylic acid isopropyl ester (Example 16): Daicel chiral pack IA 250×20 mm, MeCN:MeOH:THF:DEA, 50:50:3:0.1, 15 mL/min, 285 nm.

### Example 40

### 4-((S)-1-{4′-[(S)-2-Amino-3-((S)-3-fluoropyrrolidin-1-yl)-3-oxopropyl]-3′-fluorobiphenyl-4-yloxy}ethyl)piperidine-1-carboxylic acid isopropyl ester

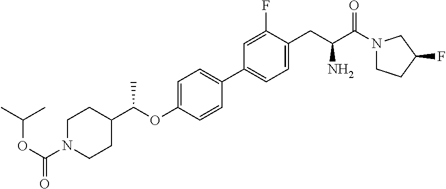

The title compound was afforded via chiral HPLC separation of (S)-4-(1-{4′-[2-amino-3-((S)-3-fluoropyrrolidin-1-yl)-3-oxopropyl]-3′-fluorobiphenyl-4-yloxy}ethyl)piperidine-1-carboxylic acid isopropyl ester (Example 16): Daicel chiral pack IA 250×20 mm, MeCN:MeOH:THF:DEA, 50:50:3:0.1, 15 mL/min, 285 nm.

### Example 41

### (S)-4-{4′-[2-Amino-3-((S)-3-fluoropyrrolidin-1-yl)-3-oxopropyl]-3′-fluorobiphenyl-4-yloxy}piperidine-1-carboxylic acid isopropyl ester p-toluenesulfonic acid salt

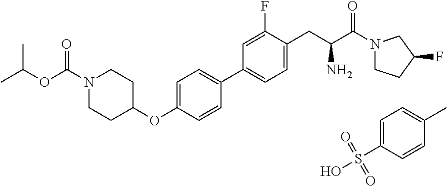

To a solution of (S)-4-{4′-[2-tert-butoxycarbonylamino-3-((S)-3-fluoropyrrolidin-1-yl)-3-oxopropyl]-3′-fluorobiphenyl-4-yloxy}piperidine-1-carboxylic acid isopropyl ester (Preparation 64, 204 mg, 0.33 mmol) in DCM (5 mL) was added TFA (1 mL) and the reaction was stirred at r.t. for 1 h. The reaction was quenched by adding sat. NaHCO₃ solution (100 mL) then the organic phase was separated and washed with brine (100 mL), then dried (MgSO₄). Removal of the solvent in vacuo afforded the title compound as the free amine. The residue was dissolved in DCM and a solution of p-toluenesulfonic acid monohydrate (1 eq, 52 mg, 0.27 mmol) in MeOH was added. Removal of the solvent in vacuo afforded the title compound as its p-toluenesulfonic acid salt: RT=3.12 min; m/z (ES⁺)=516.3 [M+H]⁺.

The following examples were prepared as their p-toluenesulfonic acid salts by treating the appropriate tert-butyl carbamate-protected amine with TFA employing the procedure outlined in Example 41.

| Ex | Structure | Name | LCMS |
|---|---|---|---|
| 42 | 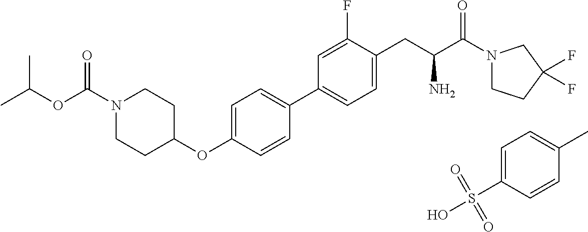 | (S)-4-{4′-[2-Amino-3- (3,3-difluoro- pyrrolidin-1-yl)-3- oxopropyl]-3′-fluoro- biphenyl-4-yloxy}- piperidine-1- carboxylic acid isopropyl ester p- toluenesulfonic acid salt | RT = 3.18 min; m/z (ES⁺) = 534.4 [M + H]⁺ |
| 43 | 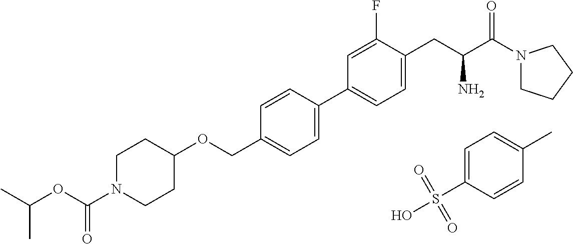 | (S)-4-[4′-(2-Amino-3- oxo-3-pyrrolidin-1- ylpropyl)-3′-fluoro- biphenyl-4- ylmethoxy]piperidine- 1-carboxylic acid isopropyl ester p- toluenesulfonic acid salt | RT = 2.87 min; m/z (ES⁺) = 512.4 [M + H]⁺ |
| 44 | 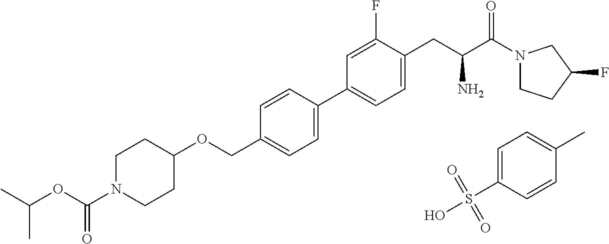 | (S)-4-{4′-[2-Amino-3- ((S)-3-fluoro- pyrrolidin-1-yl)-3- oxopropyl]-3′- fluorobiphenyl-4- ylmethoxy}piperidine- 1-carboxylic acid isopropyl ester | RT = 2.84 min; m/z (ES⁺) = 530.4 [M + H]⁺ |

### Example 45

### (S)-4-{4′-[2-Amino-3-((S)-3-fluoropyrrolidin-1-yl)-3-oxopropyl]-3′-fluorobiphenyl-3-yloxymethyl}-piperidine-1-carboxylic acid isopropyl ester hydrochloride

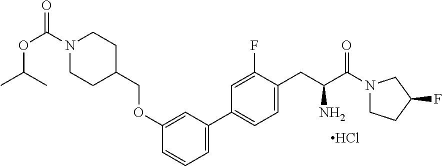

To a solution of (S)-4-{4′-[2-tert-butoxycarbonylamino-3-((S)-3-fluoropyrrolidin-1-yl)-3-oxopropyl]-3′-fluorobiphenyl-3-yloxymethyl}piperidine-1-carboxylic acid isopropyl ester (Preparation 66, 37 mg, 0.06 mmol) in DCM (5 mL) was added TFA (1 mL). The reaction was stirred at r.t. for 30 min then diluted with DCM (150 mL). Saturated NaHCO₃ solution (150 mL) was added and organics were separated, washed with brine and dried (MgSO₄). Removal of the solvent in vacuo afforded the title compound as the free amine. The product was dissolved in diethyl ether (10 mL) and treated with a few drops of HCl solution (4M in dioxane). Decanting the solvent afforded the title compound: RT=2.95 min; m/z (ES⁺)=530.4 [M+H]⁺.

The following examples were prepared by treating the appropriate tert-butyl carbamate-protected amine with TFA employing a procedure similar to that outlined in Example 45.

| Ex | Structure | Name | LCMS |
|---|---|---|---|
| 46 | 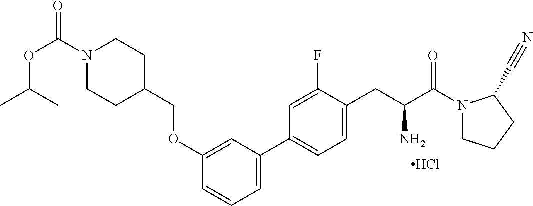 | (S)-4-{4′-[2-Amino-3- ((S)-2-cyanopyrrolidin- 1-yl)-3-oxopropyl]-3′- fluorobiphenyl-3- yloxymethyl}piperidine- 1-carboxylic acid isopropyl ester hydrochloride | RT = 2.98 min; m/z (ES⁺) = 537.4 [M + H]⁺ |
| 47 | 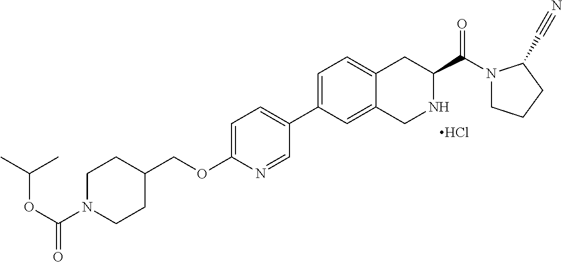 | 4-{5-[(S)-3-((S)-2- Cyanopyrrolidine-1- carbonyl)-1,2,3,4- tetrahydroisoquinolin-7- yl]pyridin-2-yloxy- methyl}piperidine-1- carboxylic acid isopropyl ester hydrochloride | RT = 2.85 min; m/z (ES⁺) = 532.4 [M + H]⁺ |
| 48 | 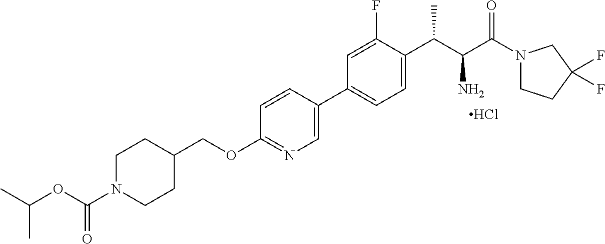 | 4-(5-{4-[1S,2S)-2- Amino-3-(3,3- difluoropyrrolidin-1-yl)- 1-methyl-3-oxopropyl]- 3-fluorophenyl}pyridin- 2-yloxymethyl) piperidine-1-carboxylic acid isopropyl ester hydrochloride | RT = 3.04 min; m/z (ES⁺) = 563.5 [M + H]⁺ |
| 49 | 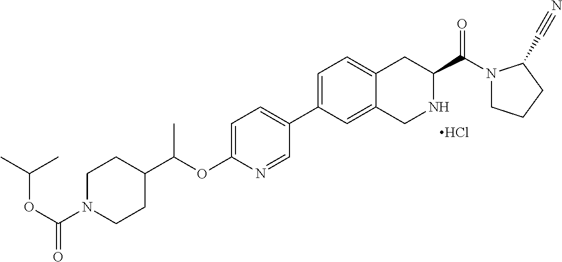 | 4-(1-{5-[(S)-3-((S)-2- Cyanopyrrolidine-1- carbonyl)-1,2,3,4- tetrahydroisoquinolin-7- yl]pyridin-2-yloxy}- ethyl)piperidine-1- carboxylic acid isopropyl ester hydrochloride | RT = 2.95 min; m/z (ES⁺) = 546.5 [M + H]⁺ |
| 50 | 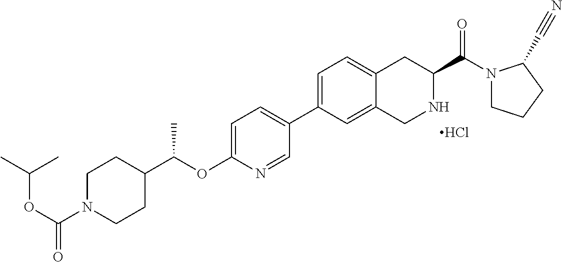 | 4-((S)-1-{5-[(S)-3-((S)- 2-Cyanopyrrolidine-1- carbonyl)-1,2,3,4- tetrahydroisoquinolin-7- yl]pyridin-2-yloxy}- ethyl)piperidine-1- carboxylic acid isopropyl ester hydrochloride | RT = 2.95 min; m/z (ES⁺) = 546.5 [M + H]⁺ |
| 51 | 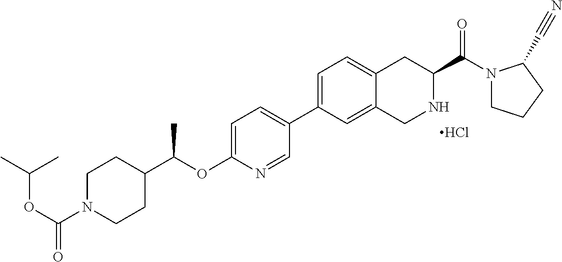 | 4-((R)-1-{5-[(S)-3-((S)- 2-Cyanopyrrolidine-1- carbonyl)-1,2,3,4- tetrahydroisoquinolin-7- yl]pyridin-2-yloxy}- ethyl)piperidine-1- carboxylic acid isopropyl ester hydrochloride | RT = 2.97 min; m/z (ES⁺) = 546.5 [M + H]⁺ |

### Example 52

### (S)-4-(4-{4-[2-Amino-3-((S)-3-fluoropyrrolidin-1-yl)-3-oxopropyl]-3-fluorophenyl}pyridin-2-yloxymethyl)piperidine-1-carboxylic acid isopropyl ester hydrochloride

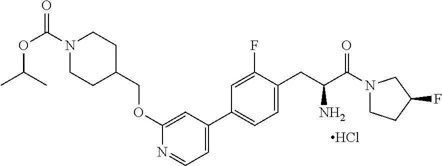

The title compound was prepared from (S)-4-(4{4-[2-tert-butoxycarbonylamino-3-((S)-3-fluoropyrrolidin-1-yl)-3-oxopropyl]-3-fluorophenyl}pyridin-2-yloxymethyl)piperidine-1-carboxylic acid isopropyl ester (Preparation 106, 150 mg, 0.24 mmol) employing a procedure similar to that outlined in Example 17: RT=2.83 min; m/z (ES⁺)=531.4 [M+H]⁺.

The biological activity of the compounds of the invention may be tested in the following assay systems:

### GPR119 Yeast Reporter Assay

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Step 3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 μg of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μL of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di((β-galactopyranoside) was used (FDG), a substrate for the enzyme that releases fluorescein, allowing a fluorimetric read-out. 20 μL per well of 500 μM FDG/2.5% Triton X100 was added (the detergent was necessary to render the cells permeable). After incubation of the cells with the substrate for 60 min, 20 μL per well of 1M sodium carbonate was added to terminate the reaction and enhance the fluorescent signal. The plates were then read in a fluorimeter at 485/535 nm.

All of Examples 1 to 52 showed activity in this assay giving an increase in fluorescent signal of at least ˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR119 was established and this cell line was used to investigate the effect of compounds of the invention on intracellular levels of cyclic AMP (cAMP). The cell monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit. Buffers and assay conditions were as described in the manufacturer's protocol.

Compounds of the invention produced a concentration-dependent increase in intracellular cAMP level and generally had an EC₅₀ of <10 μM. Compounds showing and EC₅₀ of less than 1 μM in the cAMP assay may be preferred.

### DPP-IV Assay Method

DPP-IV activity was measured by monitoring the cleavage of the fluorogenic peptide substrate, H-Gly-Pro-7-amino-4-methylcoumarin (GP-AMC) whereby the product 7-amino-4-methylcoumarin is quantified by fluorescence at excitation 380 nm and emission 460 nm. Assays were carried out in 96-well plates (Black OptiPlate-96F) in a total volume of 100 μL per well consisting of 50 mM Tris pH 7.6, 100 μM GP-AMC, 10-25 μU recombinant human DPP-IV and a range of inhibitor dilutions in a final concentration of 1% DMSO. Plates were read in a fluorimeter after 30 min incubation at 37° C. Recombinant human DPP-IV residues Asn29-Pro766 was purchased from BioMol.

All of Examples 1 to 52 showed activity in this assay having an IC₅₀ of <20 μM. Compounds of the invention of formula (Ia) generally have an IC₅₀ of <20 μM.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP Assay

HIT-T15 cells were plated in standard culture medium in 96-well plates at 100,000 cells/0.1 mL/well and cultured for 24 h and the medium was then discarded. Cells were incubated for 15 min at room temperature with 1000 stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 μL lysis buffer (5 mM HEPES, 0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μL reactions were set up containing 8 μL sample, 5 μL acceptor bead mix and 12 μL detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred.

### Insulin Secretion Assay

HIT-T15 cells are plated in standard culture medium in 12-well plates at 106 cells/1 ml/well and cultured for 3 days and the medium then discarded. Cells are washed×2 with supplemented Krebs-Ringer buffer (KRB) containing 119 mM NaCl, 4.74 mM KCl, 2.54 mM CaCl₂, 1.19 mM MgSO₄, 1.19 mM KH₂PO₄, 25 mM NaHCO₃, 10 mM HEPES at pH 7.4 and 0.1% bovine serum albumin. Cells are incubated with 1 ml KRB at 37° C. for 30 min which is then discarded. This is followed by a second incubation with KRB for 30 min, which is collected and used to measure basal insulin secretion levels for each well. Compound dilutions (0, 0.1, 0.3, 1, 3, 10 μM) are then added to duplicate wells in 1 ml KRB, supplemented with 5.6 mM glucose. After 30 min incubation at 37° C. samples are removed for determination of insulin levels. Measurement of insulin was done using the Mercodia Rat insulin ELISA kit, following the manufacturers' instructions, with a standard curve of known insulin concentrations. For each well, insulin levels are corrected by subtraction of the basal secretion level from the pre-incubation in the absence of glucose. Data is analysed using XLfit 3 software.

Compounds of the invention preferably increase insulin secretion at an EC₅₀ of less than 10 μM.

### Oral Glucose Tolerance Tests

The effects of compounds of the invention on oral glucose (Glc) tolerance may be evaluated in male Sprague-Dawley rats. Food is withdrawn 16 h before administration of Glc and remains withdrawn throughout the study. Rats have free access to water during the study. A cut is made to the animals' tails, then blood (1 drop) is removed for measurement of basal Glc levels 60 min before administration of the Glc load. Then, the rats are weighed and dosed orally with test compound or vehicle (20% aqueous hydroxypropyl-fi-cyclodextrin) 45 min before the removal of an additional blood sample and treatment with the Glc load (2 g kg⁻¹ p.o.). Blood samples are taken from the cut tip of the tail 5, 15, 30, 60, 120, and 180 min after Glc administration. Blood glucose levels are measured just after collection using a commercially available glucose-meter (OneTouch® Ultra™ from Lifescan). Compounds of the invention preferably statistically reduce the Glc excursion at doses≦100 mg kg⁻¹.

The effects of compounds of the invention on oral glucose (Glc) tolerance may also be evaluated in male C57Bl/6 or male ob/ob mice. Food is withdrawn 5 h before administration of Glc and remained withdrawn throughout the study. Mice have free access to water during the study. A cut was made to the animals' tails, then blood (20 μL) is removed for measurement of basal Glc levels 45 min before administration of the Glc load. Then, the mice are weighed and dosed orally with test compound or vehicle (20% aqueous hydroxypropyl-fi-cyclodextrin or 25% aqueous Gelucire 44/14) 30 min before the removal of an additional blood sample (20 μL) and treatment with the Glc load (2-5 g kg⁻¹ p.o.). Blood samples (20 μL) are then taken 25, 50, 80, 120, and 180 min after Glc administration. The 20 μL blood samples for measurement of Glc levels are taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood are then added to 180 μL of Trinders glucose reagent (Sigma enzymatic (Trinder) colorimetric method) in a 96-well assay plate. After mixing, the samples are left at room temperature for 30 min before being read against Glc standards (Sigma glucose/urea nitrogen combined standard set). Compounds of the invention preferably statistically reduce the Glc excursion at doses≦100 mg kg⁻¹.

1. A compound of formula (I) or a pharmaceutically acceptable salt thereof: 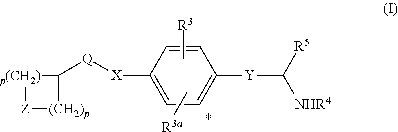
wherein:
p is 1 or 2;
when p is 2, Z is CHR¹ or NR²;
and when p is 1, Z is —N—CH₂-Ph wherein the Ph is optionally substituted by 1 or 2 groups independently selected from the group consisting of C₁₋₄ alkyl, C₁₋₄ haloalkyl and halo;
R¹ is —N(CH₃)—C(O)—O—C₂₋₄alkyl or —N(CH₃)—C(O)—O—C₃₋₆cycloalkyl wherein the cycloalkyl is optionally substituted by C₁₋₄ alkyl;
R² is —C(O)—O—C₂₋₄ alkyl, —C(O)—O—C₃₋₆ cycloalkyl wherein the cycloalkyl is optionally substituted by C₁₋₄ alkyl, —C(O)—C₂₋₄ alkyl, —C(O)—C₃₋₆ cycloalkyl wherein the cycloalkyl is optionally substituted by C₁₋₄ alkyl, or R² is: 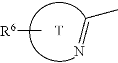
where T together with the —N═C— to which it is attached forms a 5- or 6-membered heteroaryl ring optionally containing up to 2 additional heteroatoms selected from the group consisting of N, O and S;
when T together with the —N═C— to which it is attached forms a 5-membered heteroaryl ring, R⁶ is C₂₋₄ alkyl or C₃₋₆ cycloalkyl optionally substituted by C₁₋₄ alkyl, and when T together with the —N═C— to which it is attached forms a 6-membered heteroaryl ring, R⁶ is C₂₋₄ alkyl, fluoro or chloro;
Q is —O—, —O—CR⁸H— or —CR⁸H—O—;
X is phenyl or a 5- or 6-membered heteroaryl group containing one of more heteroatoms selected from the group consisting of N, O and S; provided that when Q is —O—CR⁸H— then X is not a 6-membered heteroaryl group;
Y is a bond, —CH₂— or —CHMe-;
R³ and R^{3a} are independently selected from the group consisting of hydrogen, fluoro or chloro, or when R⁷ is cyano, R³ may be methyl; provided that when Y is a bond, and R³ and R^{3a} are in the ortho position to the Y group they are both hydrogen;
R⁴ is hydrogen or, when Y is —CH₂— or —CHMe-, R⁴ can be —CH₂— linked to position * on the phenyl ring to form a fused 6-membered N-containing heterocycle;
R⁵ is benzyl optionally substituted by one or more fluoro, chloro, cyano or methyl groups, or R⁵ is: 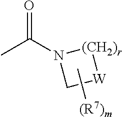
where r is 1 or 2 and m is 0, 1 or 2;
W is CH₂ or, when r is 2, W may be S;
when W is CH₂, R⁷ is fluoro or cyano, and when W is S, R⁷ is cyano; and
R⁸ is hydrogen or methyl.

2. A compound according to claim 1, or a pharmaceutically acceptable salt thereof, having the stereochemistry as defined in formula (Ia):

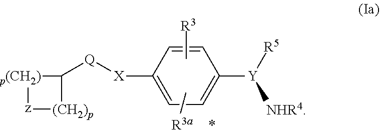

3. A compound according to claim 1, or a pharmaceutically acceptable salt thereof, wherein p is 2.

4. A compound according to claim 1, or a pharmaceutically acceptable salt thereof, wherein Z is NR².

5. A compound according to claim 4, or a pharmaceutically acceptable salt thereof, wherein R² is —C(O)OR⁴.

6. A compound according to claim 4, or a pharmaceutically acceptable salt thereof, wherein R² is: 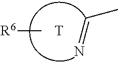
where the 5- or 6-membered heteroaryl ring formed by T together with the —N═C— to which it is attached is selected from the group consisting of oxadiazole and pyrimidine.

7. A compound according to claim 1, or a pharmaceutically acceptable salt thereof, wherein X is a meta- or para-linked phenyl or a meta- or para-linked 6-membered heteroaromatic ring containing one or two nitrogen atoms.

8. A compound according to claim 6, or a pharmaceutically acceptable salt thereof, wherein X is a para-linked phenyl or a para-linked 6-membered heteroaromatic ring containing one or two nitrogen atoms.

9. A compound according to claim 1, or a pharmaceutically acceptable salt thereof, wherein X is phenyl or pyridyl.

10. A compound according to claim 1, or a pharmaceutically acceptable salt thereof, wherein R³ is fluoro.

11. A compound according to claim 1, or a pharmaceutically acceptable salt thereof, wherein R⁴ is hydrogen.

12. A compound according to claim 1, or a pharmaceutically acceptable salt thereof, wherein R⁵ is:

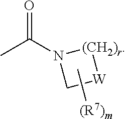

13. A compound according to claim 12, or a pharmaceutically acceptable salt thereof, wherein r is 2.

14. A compound according to claim 12, or a pharmaceutically acceptable salt thereof, wherein W is CH₂.

15. (canceled)

16. A pharmaceutical composition comprising a compound of claim 1, or a pharmaceutically acceptable salt thereof, and a pharmaceutically acceptable carrier.

17. A method for the treatment of a disease or condition in which GPR119 plays a role, said method comprising administering to a subject in need thereof an effective amount of a compound of claim 1, or a pharmaceutically acceptable salt thereof.

18. A method for the treatment of a disease or condition in which GPR119 and DPP-IV play a role, said method comprising administering to a subject in need thereof an effective amount of a compound of claim 1, or a pharmaceutically acceptable salt thereof.

19. A method for the treatment of type II diabetes, said method comprising administering to a subject in need thereof an effective amount of a compound of claim 1, or a pharmaceutically acceptable salt thereof.

20. A method for the treatment of obesity, metabolic syndrome (syndrome X), impaired glucose tolerance, hyperlipidemia, hypertriglyceridemia, hypercholesterolemia, low HDL levels or hypertension, said method comprising administering to a patient in need thereof an effective amount of a compound of claim 1, or a pharmaceutically acceptable salt thereof.

21. A compound of claim 1 selected from the group consisting of:
(S)-4-(5-{4-[2-Amino-3-(3,3-difluoropyrrolidin-1-yl)-3-oxopropyl]-3-fluorophenyl}pyridine-2-yloxy)piperidine-1-carboxylic acid isopropyl ester or a pharmaceutically acceptable salt thereof,
(S)-4-(5-{4-[2-Amino-3-((S)-2-cyanopyrrolidin-1-yl)-3-oxopropyl]-3-fluorophenyl}pyridin-2-yloxymethyl)-piperidine-1-carboxylic acid isopropyl ester or a pharmaceutically acceptable salt thereof,
(S)-4-(5-{4-[2-Amino-3-((S)-3-fluoropyrrolidin-1-yl)-3-oxopropyl]-3-fluorophenyl}pyridin-2-yloxymethyl)-piperidine-1-carboxylic acid isopropyl ester or a pharmaceutically acceptable salt thereof,
(S)-1-[2-Amino-3-(2-fluoro-4-{6-[1-(4-isopropylbenzyl)-azetidin-3-yloxy]pyridin-3-yl}phenyl)propionyl]pyrrolidine-2-carbonitrile or a pharmaceutically acceptable salt thereof,
(S)-4-{4′-[2-Amino-3-((S)-3-fluoropyrrolidin-1-yl)-3-oxopropyl]-3′-fluoro biphenyl-4-yloxymethyl}piperidine-1-carboxylic acid isopropyl ester or a pharmaceutically acceptable salt thereof,
(S)-4-{4′-[2-Amino-3-((S)-2-cyanopyrrolidin-1-yl)-3-oxopropyl]-3′-fluoro biphenyl-4-yloxymethyl}piperidine-1-carboxylic acid isopropyl ester or a pharmaceutically acceptable salt thereof,
(S)-4-[4′-(2-Amino-3-oxo-3-pyrrolidin-1-yl-propyl)-3′-fluoro biphenyl-4-yloxymethyl]-piperidine-1-carboxylic acid isopropyl ester or a pharmaceutically acceptable salt thereof,
(S)-4-(6-{-4-[2-Amino-3-((S)-3-fluoropyrrolidin-1-yl)-3-oxopropyl]-3-fluorophenyl}pyridin-3-yl oxymethyl}-piperidine-1-carboxylic acid isopropyl ester or a pharmaceutically acceptable salt thereof,
(R)-4-{4′-[1-Amino-2-((S)-2-cyanopyrrolidin-1-yl)-2-oxoethyl]biphenyl-4-yloxy}-piperidine-1-carboxylic acid isopropyl ester or a pharmaceutically acceptable salt thereof,
(S)-4-[1-(5-{4-[2-Amino-3-((S)-2-cyanopyrrolidin-1-yl)-3-oxopropyl]-3-fluorophenyl}-pyridin-2-yloxy)ethyl]-piperidine-1-carboxylic acid isopropyl ester or a pharmaceutically acceptable salt thereof,
(S)-4-(1-{5-[4-(2-Amino-3-oxo-3-pyrrolidin-1-ylpropyl)-3-fluorophenyl]pyridin-2-yloxy}ethyl)piperidine-1-carboxylic acid isopropyl ester or a pharmaceutically acceptable salt thereof,
(S)-4-[1-(5-{4-[2-Amino-3-((S)-3-fluoropyrrolidin-1-yl)-3-oxopropyl]-3-fluorophenyl}-pyridin-2-yloxy)ethyl]-piperidine-1-carboxylic acid isopropyl ester or a pharmaceutically acceptable salt thereof,
(S)-4-(5-{4-[2-Amino-3-((S)-2-cyanopyrrolidin-1-yl)-3-oxopropyl]-3-fluorophenyl}-pyridin-2-yloxy)piperidine-1-carboxylic acid isopropyl ester or a pharmaceutically acceptable salt thereof,
(S)-4-{4′-[2-Amino-3-((S)-2-cyanopyrrolidin-1-yl)-3-oxopropyl]-3′-fluorobiphenyl-4-yloxy}piperidine-1-carboxylic acid isopropyl ester or a pharmaceutically acceptable salt thereof,
(S)-4-{1-[4′-(2-Amino-3-oxo-3-pyrrolidin-1-ylpropyl)-3′-fluorobiphenyl-4-yloxy]-ethyl}piperidine-1-carboxylic acid isopropyl ester or a pharmaceutically acceptable salt thereof,
(S)-4-(1-{4′-[2-Amino-3-((S)-3-fluoropyrrolidin-1-yl)-3-oxopropyl]-3′-fluorobiphenyl-4-yloxy}ethyl)piperidine-1-carboxylic acid isopropyl ester or a pharmaceutically acceptable salt thereof,
(S)-4-(5-{4-[2-Amino-3-((S)-3-fluoropyrrolidin-1-yl)-3-oxopropyl]-3-fluorophenyl}pyridin-2-yloxy)piperidine-1-carboxylic acid isopropyl ester hydrochloride or a free base thereof,
(S)-2-Amino-3-(2-fluoro-4-{6-[1-(4-isopropylbenzyl)azetidin-3-yloxy]pyridin-3-yl}phenyl)-1-pyrrolidin-1-yl propan-1-one p-toluenesulfonic acid salt or a free base thereof,
(S)-4-{6-[4-(2-Amino-3-oxo-3-pyrrolidin-1-yl propyl)-3-fluoro-phenyl]pyridin-3-yloxymethyl}piperidine-1-carboxylic acid isopropyl ester or a pharmaceutically acceptable salt thereof,
(S)-4-(6-{4-[2-Amino-3-((S)-2-cyanopyrrolidin-1-yl)-3-oxopropyl]-3-fluorophenyl}pyridin-3-yloxymethyl)piperidine-1-carboxylic acid isopropyl ester or a pharmaceutically acceptable salt thereof,
(S)-4-{4′-[1-Amino-2-((S)-3-fluoropyrrolidin-1-yl)-2-oxoethyl]biphenyl-4-yloxy}piperidine-1-carboxylic acid isopropyl ester or a pharmaceutically acceptable salt thereof,
4-[(R)-1-(5-{4-[(S)-2-Amino-3-((S)-2-cyanopyrrolidin-1-yl)-3-oxopropyl]-3-fluorophenyl}pyridin-2-yloxy)ethyl]piperidine-1-carboxylic acid isopropyl ester or a pharmaceutically acceptable salt thereof,
4-[(S)-1-(5-{4-[(S)-2-Amino-3-((S)-2-cyanopyrrolidin-1-yl)-3-oxopropyl]-3-fluorophenyl}pyridin-2-yloxy)ethyl]-piperidine-1-carboxylic acid isopropyl ester or a pharmaceutically acceptable salt thereof,
4-((R)-1-{5-[4-((S)-2-Amino-3-oxo-3-pyrrolidin-1-ylpropyl)-3-fluorophenyl]-pyridin-2-yloxy}ethyl)-piperidine-1-carboxylic acid isopropyl ester or a pharmaceutically acceptable salt thereof,
4-((S)-1-{5-[4-((S)-2-Amino-3-oxo-3-pyrrolidin-1-ylpropyl)-3-fluorophenyl]-pyridin-2-yloxy}-ethyl)piperidine-1-carboxylic acid isopropyl ester or a pharmaceutically acceptable salt thereof,
4-[(R)-1-(5-{4-[(S)-2-Amino-3-((S)-3-fluoropyrrolidin-1-yl)-3-oxopropyl]-3-fluoro-phenyl}pyridin-2-yloxy)ethyl]piperidine-1-carboxylic acid isopropyl ester or a pharmaceutically acceptable salt thereof,
4-[(S)-1-(5-{4-[(S)-2-Amino-3-((S)-3-fluoropyrrolidin-1-yl)-3-oxopropyl]-3-fluoro-phenyl}pyridin-2-yloxy)ethyl]piperidine-1-carboxylic acid isopropyl ester or a pharmaceutically acceptable salt thereof,
(S)-4-{4′-[2-Amino-3-((S)-2-cyanopyrrolidin-1-yl)-3-oxopropyl]biphenyl-4-yloxymethyl}piperidine-1-carboxylic acid isopropyl ester hydrochloride or a free base thereof,
(S)-4-{4′-[2-Amino-3-((S)-3-fluoropyrrolidin-1-yl)-3-oxopropyl]biphenyl-4-yloxymethyl}piperidine-1-carboxylic acid isopropyl ester hydrochloride or a free base thereof,
2-Amino-3-(2-fluoro-4-{6-[1-(6-methylpyrazin-2-yl)piperidin-4-ylmethoxy]pyridin-3-yl}phenyl)-1-pyrrolidin-1-yl-propan-1-one or a pharmaceutically acceptable salt thereof,
1-(4-{5-[4-(2-Amino-3-oxo-3-pyrrolidin-1-ylpropyl)-3-fluorophenyl]pyridin-2-yloxymethyl}piperidin-1-yl)-3-methylbutan-1-one or a pharmaceutically acceptable salt thereof,
(4-{5-[4-(2-Amino-3-oxo-3-pyrrolidin-1-ylpropyl)-3-fluorophenyl]pyridin-2-yloxy}cyclohexyl)methylcarbamic acid isopropyl ester or a pharmaceutically acceptable salt thereof,
2-Amino-3-{2-fluoro-4-[6-(5′-methyl-3,4,5,6-tetrahydro-2H-[1,2′]bipyridinyl-4-yloxy)pyridin-3-yl]phenyl]phenyl}-1-pyrrolidin-1-yl propan-1-one or a pharmaceutically acceptable salt thereof,
(S)-2-Amino-3-(2-fluoro-4-{6-[1-(3-isopropyl-[1,2,4]oxadiazol-5-yl)piperidin-4-ylmethoxy]pyridine-3-yl}phenyl)-1-pyrrolidin-1-yl-propan-1-one or a pharmaceutically acceptable salt thereof,
(S)-1-(4-{5-[4-(2-Amino-3-oxo-3-pyrrolidin-1-ylpropyl)-3-fluorophenyl]pyridin-2-yloxymethyl}piperidin-1-yl)-3-methylbutan-1-one or a pharmaceutically acceptable salt thereof,
(S)-4-{5-[4-(2-Amino-3-oxo-3-pyrrolidin-1-ylpropyl)-3-fluorophenyl]pyridine-2-yloxymethyl}piperidine-1-carboxylic acid isopropyl ester or a pharmaceutically acceptable salt thereof,
4-{(S)-1-[4′-((S)-2-Amino-3-oxo-3-pyrrolidin-1-ylpropyl)-3′-fluorobiphenyl-4-yloxy]ethyl}piperidine-1-carboxylic acid isopropyl ester or a pharmaceutically acceptable salt thereof,
4-{(R)-1-[4′-((S)-2-Amino-3-oxo-3-pyrrolidin-1-ylpropyl)-3′-fluorobiphenyl-4-yloxy]ethyl}piperidine-1-carboxylic acid isopropyl ester or a pharmaceutically acceptable salt thereof,
4-((R)-1-{4′-[(S)-2-Amino-3-((S)-3-fluoropyrrolidin-1-yl)-3-oxopropyl]-3′-fluorobiphenyl-4-yloxy}ethyl)piperidine-1-carboxylic acid isopropyl ester or a pharmaceutically acceptable salt thereof,
4-((S)-1-{4′-[(S)-2-Amino-3-((S)-3-fluoropyrrolidin-1-yl)-3-oxopropyl]-3′-fluorobiphenyl-4-yloxy}ethyl)piperidine-1-carboxylic acid isopropyl ester or a pharmaceutically acceptable salt thereof,
(S)-4-{4′-[2-Amino-3-((S)-3-fluoropyrrolidin-1-yl)-3-oxopropyl]-3′-fluorobiphenyl-4-yloxy}piperidine-1-carboxylic acid isopropyl ester p-toluenesulfonic acid salt or a free base thereof,
(S)-4-{4′-[2-Amino-3-(3,3-difluoro-pyrrolidin-1-yl)-3-oxopropyl]-3′-fluoro-biphenyl-4-yloxy}-piperidine-1-carboxylic acid isopropyl ester p-toluenesulfonic acid salt or a free base thereof,
(S)-4-[4′-(2-Amino-3-oxo-3-pyrrolidin-1-ylpropyl)-3′-fluoro-biphenyl-4-ylmethoxy]piperidine-1-carboxylic acid isopropyl ester p-toluenesulfonic acid salt or a free base thereof,
(S)-4-{4′-[2-Amino-3-((S)-3-fluoro-pyrrolidin-1-yl)-3-oxopropyl]-3′-fluorobiphenyl-4-ylmethoxy}piperidine-1-carboxylic acid isopropyl ester or a pharmaceutically acceptable salt thereof,
(S)-4-{4′-[2-Amino-3-((S)-3-fluoropyrrolidin-1-yl)-3-oxopropyl]-3′-fluorobiphenyl-3-yloxymethyl}piperidine-1-carboxylic acid isopropyl ester hydrochloride or a free base thereof,
(S)-4-{4′-[2-Amino-3-((S)-2-cyanopyrrolidin-1-yl)-3-oxopropyl]-3′-fluorobiphenyl-3-yloxymethyl}piperidine-1-carboxylic acid isopropyl ester hydrochloride or a free base thereof,
4-{5-[(S)-3-((S)-2-Cyanopyrrolidine-1-carbonyl)-1,2,3,4-tetrahydroisoquinolin-7-yl]pyridin-2-yloxy-methyl}piperidine-1-carboxylic acid isopropyl ester hydrochloride or a free base thereof,
4-(5-{4-[1S,2S)-2-Amino-3-(3,3-difluoropyrrolidin-1-yl)-1-methyl-3-oxopropyl]-3-fluorophenyl}pyridin-2-yloxymethyl)piperidine-1-carboxylic acid isopropyl ester hydrochloride or a free base thereof,
4-(1-{5-[(S)-3-((S)-2-Cyanopyrrolidine-1-carbonyl)-1,2,3,4-tetrahydroisoquinolin-7-yl]pyridin-2-yloxy}-ethyl)piperidine-1-carboxylic acid isopropyl ester hydrochloride or a free base thereof,
4-((S)-1-{5-[(S)-3-((S)-2-Cyanopyrrolidine-1-carbonyl)-1,2,3,4-tetrahydroisoquinolin-7-yl]pyridin-2-yloxy}-ethyl)piperidine-1-carboxylic acid isopropyl ester hydrochloride or a free base thereof,
4-((R)-1-{5-[(S)-3-((S)-2-Cyanopyrrolidine-1-carbonyl)-1,2,3,4-tetrahydroisoquinolin-7-yl]pyridin-2-yloxy}-ethyl)piperidine-1-carboxylic acid isopropyl ester hydrochloride or a free base thereof, and
(S)-4-(4-{-4-[2-Amino-3-((S)-3-fluoropyrrolidin-1-yl)-3-oxopropyl]-3-fluorophenyl}pyridin-2-yloxymethyl)piperidine-1-carboxylic acid isopropyl ester hydrochloride or a free base thereof.
